SEQUENCE LISTING (138 sequences)

SEQ ID NO: 1 - DNA, 5562 bp, Homo sapiens
  source 1..5562: mol_type = unassigned DNA
  CDS 190..2319
cagagggctgctggctggctaagtccctcccgctcccggctctcgcctcactaggagcggctctcggtgcagcgggacagggcgaagcggcctgcgcccacggagcgcgcgacactgcccggaagggaccgccacccttgccccctcagctgcccactcgtgatttccagcggcctccgcgcgcgcacgatgccctcggccaccagccacagcgggagcggcagcaagtcgtccggaccgccaccgccgtcgggttcctccgggagtgaggcggccgcgggagccggggccgccgcgccggcttctcagcaccccgcaaccggcaccggcgctgtccagaccgaggccatgaagcagattctcggggtgatcgacaagaaacttcggaacctggagaagaaaaagggtaagcttgatgattaccaggaacgaatgaacaaaggggaaaggcttaatcaagatcagctggatgccgtttctaagtaccaggaagtcacaaataatttggagtttgcaaaagaattacagaggagtttcatggcactaagtcaagatattcagaaaacaataaagaagacagcacgtcgggagcagcttatgagagaagaagctgaacagaaacgtttaaaaactgtacttgagctacagtatgttttggacaaattgggagatgatgaagtgcggactgacctgaaacaaggtttgaatggagtgccaatattgtccgaagaggagttgtcattgttggatgaattctataagctagtagaccctgaacgggacatgagcttgaggttgaatgaacagtatgaacatgcctccattcacctgtgggacctgctggaagggaaggaaaaacctgtatgtggaaccacctataaagttctaaaggaaattgttgagcgtgtttttcagtcaaactactttgacagcacccacaaccaccagaatgggctgtgtgaggaagaagaggcagcctcagcacctgcagttgaagaccaggtacctgaagctgaacctgagccagcagaagagtacactgagcaaagtgaagttgaatcaacagagtatgtaaatagacagttcatggcagaaacacagttcaccagtggtgaaaaggagcaggtagatgagtggacagttgaaacggttgaggtggtaaattcactccagcagcaacctcaggctgcatccccttcagtaccagagccccactctttgactccagtggctcaggcagatccccttgtgagaagacagcgagtacaagaccttatggcacaaatgcagggtccctataatttcatacaggattcaatgctggattttgaaaatcagacacttgatcctgccattgtatctgcacagcctatgaatccaacacaaaacatggacatgccccagctggtttgccctccagttcattctgaatctagacttgctcagcctaatcaagttcctgtacaaccagaagcgacacaggttcctttggtatcatccacaagtgaggggtacacagcatctcaacccttgtaccagccttctcatgctacagagcaacgaccacagaaggaaccaattgatcagattcaggcaacaatctctttaaatacagaccagactacagcatcatcatcccttcctgctgcgtctcagcctcaagtatttcaggctgggacaagcaaacctttacatagcagtggaatcaatgtaaatgcagctccattccaatccatgcaaacggtgttcaatatgaatgccccagttcctcctgttaatgaaccagaaactttaaaacagcaaaatcagtaccaggccagttataaccagagcttttctagtcagcctcaccaagtagaacaaacagagcttcagcaagaacagcttcaaacagtggttggcacttaccatggttccccagaccagtcccatcaagtgactggtaaccaccagcagcctcctcagcagaacactggatttccacgtagcaatcagccctattacaatagtcgtggtgtgtctcgtggaggctcccgtggtgctagaggcttgatgaatggataccggggccctgccaatggattcagaggaggatatgatggttaccgcccttcattctctaacactccaaacagtggttatacacagtctcagttcagtgctccccgggattactctggctatcaacgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaatcgccaaaaacacactggccagtgtaccataatatgttaccagaagagttattatctatttgttctccctttcaggaaacttattgtaaagggactgttttcatcccataaagacaggactacaattgtcagctttctattacctggatatggaaggaaactatttttactctgcatgttctgtcctaagcgtcatcttgagccttgcacatgatactcagattcctcacccttgcttaggagtaaaacaatatactttacagggtgataataatctccatagttatttgaagtggcttgaaaaaggcaagattgacttttatgacattggataaaatctacaaatcagccctcgagttattcaatgataactgacaaactaaattatttccctagaaaggaagatgaaaggagtggagtgtggtttggcagaacaactgcatttcacagcttttccagttaaattggagcactgaacgttcagatgcataccaaattatgcatgggtcctaatcacacatataaggctggctaccagctttgacacagcactgttcatctggccaaacaactgtggttaaaaacacatgtaaaatgctttttaacagctgatactgtataagacaaagccaagatgcaaaattaggctttgattggcactttttgaaaaatatgcaacaaatatgggatgtaatccggatggccgcttctgtacttaatgtgaaatatttagatacctttttgaacacttaacagtttctttgagacaatgacttttgtaaggattggtactatctatcattccttatgacatgtacattgtctgtcactaatccttggattttgctgtattgtcacctaaattggtacaggtactgatgaaaatctctagtggataatcataacactctcggtcacatgtttttccttcagcttgaaagcttttttttaaaaggaaaagataccaaatgcctgctgctaccacccttttcaattgctatcttttgaaaggcaccagtatgtgttttagattgatttccctgtttcagggaaatcacggacagtagtttcagttctgatggtataagcaaaacaaataaaacgtttataaaagttgtatcttgaaacactggtgttcaacagctagcagcttatgtgattcaccccatgccacgttagtgtcacaaattttatggtttatctccagcaacatttctctagtacttgcacttattatcttttgtctaatttaaccttaactgaattctccgtttctcctggaggcatttatattcagtgataattccttcccttagatgcatagggagagtctctaaatttgatggaaatggacacttgagtagtgacttagccttatgtactctgttggaatttgtgctagcagtttgagcactagttctgtgtgcctaggaagttaatgctgcttattgtctcattctgacttcatggagaattaatcccacctttaagcaaaggctactaagttaatggtattttctgtgcagaaattaaattttattttcagcatttagcccaggaattcttccagtaggtgctcagctatttaaaaacaaaactattctcaaacattcatcattagacaactggagtttttgctggttttgtaacctaccaaaatggataggctgttgaacattccacattcaaaagttttgtagggtggtgggaaatgggggatcttcaatgtttattttaaaataaaataaaataagttcttgacttttctcatgtgtggttgtggtacatcatattggaagggttaacctgttactttggcaaatgagtatttttttgctagcacctccccttgcgtgctttaaatgacatctgcctgggatgtaccacaaccatatgttacctgtatcttaggggaatggataaaatatttgtggtttactgggtaatccctagatgatgtatgcttgcagtcctatataaaactaaatttgctatctgtgtagaaaataatttcatgacatttacaatcaggactgaagtaagttcttcacacagtgacctctgaatcagtttcagagaagggatgggggagaaaatgccttctaggttttgaacttctatgcattagtgcagatgttgtgaatgtgtaaaggtgttcatagtttgactgtttctatgtatgttttttcaaagaattgttcctttttttgaactataatttttctttttttggttattttaccatcacagtttaaatgtatatcttttatgtctctactcagaccatatttttaaaggggtgcctcattatggggcagagaacttttcaataagtctcattaagatctgaatcttggttctaagcattctgtataatatgtgattgcttgtcctagctgcagaaggccttttgtttggtcaaatgcatattttagcagagtttcaaggaaatgattgtcacacatgtcactgtagcctcttggtgtagcaagctcacatacaaaatacttttgtatatgcataatataaatcatctcatgtggatatgaaacttcttttttaaaacttaaaaaggtagaatgttattgattaccttgattagggcagttttatttccagatcctaataattcctaaaaaatatggaaaagttttttttcaatcattgtaccttgatattaaaacaaatatcctttaagtatttctaatcagttagcttctacagttcttttgtctccttttatatgcagctcttacgtgggagacttttccacttaaaggagacatagaatgtgtgcttattctcagaaggttcattaactgaggtgatgagttaacaactagttgagcagtcagcttcctaagtgttttaggacatttgttcattatattttccgtcatataactagaggaagtggaatgcagataagtgccgaattcaaacccttcattttatgtttaagctcctgaatctgcattccacttgggttgtttttaagcattctaaattttagttgattataagttagatttcacagaatcagtattgcccttgatcttgtcctttttatggagttaacggggaggaagacccctcaggaaaacgaaagtaaattgttaaggctcatcttcatacctttttccattttgaatcctacaaaaatactgcaaaagactagtgaatgtttaaaattacactagattaaataatatgaaagtc

SEQ ID NO: 2 - protein, 709 aa, Homo sapiens
MPSATSHSGSGSKSSGPPPPSGSSGSEAAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLVDPERDMSLRLNEQYEHASIHLWDLLEGKEKPVCGTTYKVLKEIVERVFQSNYFDSTHNHQNGLCEEEEAASAPAVEDQVPEAEPEPAEEYTEQSEVESTEYVNRQFMAETQFTSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTELQQEQLQTVVGTYHGSPDQSHQVTGNHQQPPQQNTGFPRSNQPYYNSRGVSRGGSRGARGLMNGYRGPANGFRGGYDGYRPSFSNTPNSGYTQSQFSAPRDYSGYQRDGYQQNFKRGSGQSGPRGAPRGRGGPPRPNRGMPQMNTQQVN

SEQ ID NO: 3 - DNA, 3553 bp, Homo sapiens
  source 1..3553: mol_type = unassigned DNA
  CDS 190..2274
cagagggctgctggctggctaagtccctcccgctcccggctctcgcctcactaggagcggctctcggtgcagcgggacagggcgaagcggcctgcgcccacggagcgcgcgacactgcccggaagggaccgccacccttgccccctcagctgcccactcgtgatttccagcggcctccgcgcgcgcacgatgccctcggccaccagccacagcgggagcggcagcaagtcgtccggaccgccaccgccgtcgggttcctccgggagtgaggcggccgcgggagccggggccgccgcgccggcttctcagcaccccgcaaccggcaccggcgctgtccagaccgaggccatgaagcagattctcggggtgatcgacaagaaacttcggaacctggagaagaaaaagggtaagcttgatgattaccaggaacgaatgaacaaaggggaaaggcttaatcaagatcagctggatgccgtttctaagtaccaggaagtcacaaataatttggagtttgcaaaagaattacagaggagtttcatggcactaagtcaagatattcagaaaacaataaagaagacagcacgtcgggagcagcttatgagagaagaagctgaacagaaacgtttaaaaactgtacttgagctacagtatgttttggacaaattgggagatgatgaagtgcggactgacctgaaacaaggtttgaatggagtgccaatattgtccgaagaggagttgtcattgttggatgaattctataagctagtagaccctgaacgggacatgagcttgaggttgaatgaacagtatgaacatgcctccattcacctgtgggacctgctggaagggaaggaaaaacctgtatgtggaaccacctataaagttctaaaggaaattgttgagcgtgtttttcagtcaaactactttgacagcacccacaaccaccagaatgggctgtgtgaggaagaagaggcagcctcagcacctgcagttgaagaccaggtacctgaagctgaacctgagccagcagaagagtacactgagcaaagtgaagttgaatcaacagagtatgtaaatagacagttcatggcagaaacacagttcaccagtggtgaaaaggagcaggtagatgagtggacagttgaaacggttgaggtggtaaattcactccagcagcaacctcaggctgcatccccttcagtaccagagccccactctttgactccagtggctcaggcagatccccttgtgagaagacagcgagtacaagaccttatggcacaaatgcagggtccctataatttcatacaggattcaatgctggattttgaaaatcagacacttgatcctgccattgtatctgcacagcctatgaatccaacacaaaacatggacatgccccagctggtttgccctccagttcattctgaatctagacttgctcagcctaatcaagttcctgtacaaccagaagcgacacaggttcctttggtatcatccacaagtgaggggtacacagcatctcaacccttgtaccagccttctcatgctacagagcaacgaccacagaaggaaccaattgatcagattcaggcaacaatctctttaaatacagaccagactacagcatcatcatcccttcctgctgcgtctcagcctcaagtatttcaggctgggacaagcaaacctttacatagcagtggaatcaatgtaaatgcagctccattccaatccatgcaaacggtgttcaatatgaatgccccagttcctcctgttaatgaaccagaaactttaaaacagcaaaatcagtaccaggccagttataaccagagcttttctagtcagcctcaccaagtagaacaaacagagcttcagcaagaacagcttcaaacagtggttggcacttaccatggttccccagaccagtcccatcaagtgactggtaaccaccagcagcctcctcagcagaacactggatttccacgtagcaatcagccctattacaatagtcgtggtgtgtctcgtggaggctcccgtggtgctagaggcttgatgaatggataccggggccctgccaatggattcagaggaggatatgatggttaccgcccttcattctctaacactccaaacagtggttatacacagtctcagttcagtgctccccgggattactctggctatcaacgggatggatatcagcagaatttcaagcgaggctctgggcagagtggaccacggggagccccacgaggtaatattttgtggtggtgatcctagctcctaagtggagcttctgttctggccttggaagagctgttaatagtctgcatgttaggaatacatttatcctttccagacttgttgctagggattaaatgaaatgctctgtttctaaaacttaatcttggacccaaattttaatttttgaatgatttaattttccctgttactatataaactgtcttgaaaactagaacatattctcttctcagaaaaagtgtttttccaactgaaaattatttttcaggtcctaaaacctgctaaatgtttttaggaagtacttactgaaacatttttgtaagacatttttggaatgagattgaacatttatataaatttattattcctctttcatttttttgaaacatgcctattatattttagggccagacaccctttaatggccggataagccatagttaacatttagagaaccatttagaagtgatagaactaatggaatttgcaatgccttttggacctctattagtgatataaatatcaagttatttctgacttttaaacaaaactcccaaattcctaacttattgagctatacttaaaaaaaattacaggtttagagagttttttgtttttcttttactgttggaaaactacttcccattttggcaggaagttaacctatttaacaattagagctagcatttcatgtagtctgaaattctaaatggttctctgatttgagggaggttaaacatcaaacaggtttcctctattggccataacatgtataaaatgtgtgttaaggaggaattacaacgtactttgatttgaatactagtagaaactggccaggaaaaaggtacatttttctaaaaattaatggatcacttgggaattactgacttgactagaagtatcaaaggatgtttgcatgtgaatgtgggttatgttctttcccaccttgtagcatattcgatgaaagttgagttaactgatagctaaaaatctgttttaacagcatgtaaaaagttattttatctgttaaaagtcattatacagttttgaatgttatgtagtttctttttaacagtttaggtaataaggtctgttttcattctggtgcttttattaattttgatagtatgatgttacttactactgaaatgtaagctagagtgtacactagaatgtaagctccatgagagcaggtaccttgtctgtcttctctgctgtatctattcccaacgcttgatgatggtgcctggcacatagtaggcactcaataaatatttgttgaatgaatgaa

SEQ ID NO: 4 - protein, 694 aa, Homo sapiens
MPSATSHSGSGSKSSGPPPPSGSSGSEAAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLVDPERDMSLRLNEQYEHASIHLWDLLEGKEKPVCGTTYKVLKEIVERVFQSNYFDSTHNHQNGLCEEEEAASAPAVEDQVPEAEPEPAEEYTEQSEVESTEYVNRQFMAETQFTSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTELQQEQLQTVVGTYHGSPDQSHQVTGNHQQPPQQNTGFPRSNQPYYNSRGVSRGGSRGARGLMNGYRGPANGFRGGYDGYRPSFSNTPNSGYTQSQFSAPRDYSGYQRDGYQQNFKRGSGQSGPRGAPRGNILWW

SEQ ID NO: 5 - DNA, 1605 bp, Canis lupus
  source 1..1605: mol_type = unassigned DNA; note = Canis familiaris
  CDS 46..1392
gtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagagggatgccgcaaatgaacactcagcaagtgaattaatctgattcacaggattatgtttaaacgccaaaaacacactggccagtgtaccataatatgttaccagaagagttattatctatttgttctccctttcaggaaacttattgtaaagggactgttttcatcccataaagacaggactacaattgtcagctttatattacctggaaaaaaaaaaaaaaaaaaaaaa

SEQ ID NO: 6 - protein, 448 aa, Canis lupus
  source 1..448: note = Canis familiaris
MALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKSVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTEGCRK

SEQ ID NO: 7 - DNA, 4154 bp, Canis lupus
  source 1..4154: mol_type = unassigned DNA; note = Canis familiaris
  CDS 1..2154
atgccgtcggccaccagcctcagcggaagcggcagcaagtcgtcgggcccgccgcccccgtcgggttcctccgggagcgaggcggcggcggcggcgggggcggcgggggcggcgggggccggggcggctgcgcccgcctcccagcaccccgcgaccggcaccggcgctgtccagaccgaggccatgaagcagatcctcggggtgatcgacaagaaactccggaacctggagaagaaaaagggcaagcttgatgattaccaggaacgaatgaacaaaggggaaaggcttaatcaagatcagctggatgccgtatctaagtaccaggaagtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagaccttcagcaagaacagcttcaaacagtggttggcacttaccatggttcccaggaccagccccaccaagtgactggtaaccatcagcagcctccccagcagaacactggatttccacgtagcagtcagccctattacaatagtcgtggtgtgtctcgtggtggttcccgtggtgctagaggcttaatgaatggatacaggggccctgccaatggattcagaggaggatatgatggttaccgcccttcattctctaacactccaaacagtggttatacacagtctcagttcagtgctccccgggactactctggctatcagcgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaaacgccaaaaacacactggccagtgtaccataatatgttaccagaagagttattatctatttgttctccctttcaggaaacttattgtaaagggactgttttcatcccataaagacaggactacaattgtcagctttatattacctggatatggaaggaaactatttttattctgcatgttcttcctaagcgtcatcttgagccttgcacatgatactcagattcctcacccttgcttaggagtaaaacataatacactttacagggtgatatctccatagttatttgaagtggcttggaaaaagcaagattaacttctgacattggataaaaatcaacaaatcagccctagagttattcaaatggtaattgacaaaaactaaaatatttcccttcgagaaggagtggaatgtggtttggcagaacaactgcatttcacagcttttccggttaaattggagcactaaacgtttagatgcataccaaattatgcatgggcccttaatataaaaggctggctaccagctttgacacagcactattcatcctctggccaaacaactgtggttaaacaacacatgtaaattgctttttaacagctgatactataataagacaaagccaaaatgcaaaaattgggctttgattggcactttttgaaaaatatgcaacaaatatgggatgtaatctggatggccgcttctgtacttaatgtgaagtatttagatacctttttgaacacttaacagtttcttctgacaatgacttttgtaaggattggtactatctatcattccttataatgtacattgtctgtcactaatcctcagatcttgctgtattgtcacctaaattggtacaggtactgatgaaaatatctaatggataatcataacactcttggtcacatgtttttcctgcagcctgaaggtttttaaaagaaaaagatatcaaatgcctgctgctaccacccttttaaattgctatcttttgaaaagcaccagtatgtgttttagattgatttccctattttagggaaatgacagacagtagtttcagttctgatggtataagcaaaacaaataaaacatgtttataaaagttgtatcttgaaacactggtgttcaacagctagcagcttatgtggttcaccccatgcattgttagtgtttcagattttatggttatctccagcagctgtttctgtagtacttgcatttatcttttgtctaaccctaatattctcacggaggcatttatattcaaagtggtgatcccttcacttagacgcatagggagagtcacaagtttgatgaagaggacagtgtagtaatttatatgctgttggaatttgtgctagcagtttgagcactagttctgtgtgcctatgaacttaatgctgcttgtcatattccactttgacttcatggagaattaatcccatctactcagcaaaggctatactaatactaagttaatggtattttctgtgcagaaattgaattttgttttattagcatttagctaaggaatttttccagtaggtgctcagctactaaagaaaaacaaaaacaagacacaaaactattctcaaacattcattgttagacaactggagtttttgctggttttgtaacctactaaaatggataggctgttgaacattccacattcaaaagttttttgtagggtggtggggaagggggggtgtcttcaatgtttattttaaaataaaataagttcttgacttttctcatgtgtggttgtggtacatcatattggaagggttatctgtttacttttgcaaatgagtatttctcttgctagcacctcccgttgtgcgctttaaatgacatctgcctgggatgtaccacaaccatatgttagctgtattttatggggaatagataaaatattcgtggtttattgggtaatccctagatgtgtatgcttacaatcctatatataaaactaaat

SEQ ID NO: 8 - protein, 717 aa, Canis lupus
  source 1..717: note = Canis familiaris
MPSATSLSGSGSKSSGPPPPSGSSGSEAAAAAGAAGAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKSVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTDLQQEQLQTVVGTYHGSQDQPHQVTGNHQQPPQQNTGFPRSSQPYYNSRGVSRGGSRGARGLMNGYRGPANGFRGGYDGYRPSFSNTPNSGYTQSQFSAPRDYSGYQRDGYQQNFKRGSGQSGPRGAPRGRGGPPRPNRGMPQMNTQQVN

SEQ ID NO: 9 - DNA, 4939 bp, Canis lupus
  source 1..4939: mol_type = unassigned DNA; note = Canis familiaris
  CDS 1..2109
atgccgtcggccaccagcctcagcggaagcggcagcaagtcgtcgggcccgccgcccccgtcgggttcctccgggagcgaggcggcggcggcggcgggggcggcgggggcggcgggggccggggcggctgcgcccgcctcccagcaccccgcgaccggcaccggcgctgtccagaccgaggccatgaagcagatcctcggggtgatcgacaagaaactccggaacctggagaagaaaaagggcaagcttgatgattaccaggaacgaatgaacaaaggggaaaggcttaatcaagatcagctggatgccgtatctaagtaccaggaagtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagaccttcagcaagaacagcttcaaacagtggttggcacttaccatggttcccaggaccagccccaccaagtgactggtaaccatcagcagcctccccagcagaacactggatttccacgtagcagtcagccctattacaatagtcgtggtgtgtctcgtggtggttcccgtggtgctagaggcttaatgaatggatacaggggccctgccaatggattcagaggaggatatgatggttaccgcccttcattctctaacactccaaacagtggttatacacagtctcagttcagtgctccccgggactactctggctatcagcgggatggatatcagcagaatttcaagcgaggctctgggcagagtggaccacggggagccccacgaggtaatattttgtggtggtgatcctagctcctaagtggagcttctgttctggccttggaagagctgttccatagtctgcatgtaggttacatgttaggaatacatttatcattaccagacttgttgctagggattaaatgaaatgctctgtttctaaaacttctcttgaacccaaatttaattttttgaatgactttccctgttactatataaattgtcttgaaaactagaacatttctcctcctcagaaaaagtgtttttccaactgcaaattatttttcaggtcctaaaacctgctaaatgtttttaggaagtacttactgaaacatttttgtaagacatttttggaatgagattgaacatttatataaatttattattattcctctttcatttttgaacatgcatattatattttagggtcagaaatcctttaatggccaaataagccatagttacatttagagaaccatttagaagtgatagaactaactgaaatttcaatgcctttggatcattaatagcgatataaatttcaaattgtttctgacttttaaataaaacatccaaaatcctaactaacttcctgaactatatttaaaaattacaggtttaaggagtttctggttttttttctcttaccataggaaaactgtttcctgtttggccaggaagtcaacctgtgtaataattagaagtagcatttcatatgatctgaagttctaaatggttctctgatttaagggaagttaaattgaataggtttcctctagttattggccataacatgtataaaatgtatattaaggaggaatacaaagtactttgatttcaatgctagtagaaactggccagcaaaaaggtgcattttatttttaaattaatggatcacttgggaattactgacttgaagtatcaaaggatatttgcatgtgaatgtgggttatgttctttctcaccttgtagcatattctatgaaagttgagttgactggtagctaaaaatctgttttaacagcatgtaaaaagttattttatctgttacaagtcattatacaattttgaatgttatgtagtttctttttaacagtttaggtaacaaggtctgtttttcattctggtgcttttattaattttgatagtatgatgttacttactactgaaatgtaagctagagtgtacactagaatgtaagctccatgagagcaggtaccttgtctgtcttcactgctgtatctatttccaacgcctgatgacagtgcctgacacatagtaggcactcaataaatacttgttgaatgaatgaatgaatgagtactggtggaatactccattagctctactcttcttttagctagagaacatgagcaaatttgcgcatgacaacttccaggacaggtgaacactgaagaattgacctcttaaacctaataatgtggtgacaagctgcccacatgcttcttgacttcagatgaaaatctgcttgaaggcaaagcaaataatatttgaaagaaaaaccaaatgccatttttgtcttctaggtcgtggagggcccccaagacccaacagagggatgccgcaaatgaacactcagcaagtgaattaatctgattcacaggattatgtttaaacgccaaaaacacactggccagtgtaccataatatgttaccagaagagttattatctatttgttctccctttcaggaaacttattgtaaagggactgttttcatcccataaagacaggactacaattgtcagctttatattacctggatatggaaggaaactatttttattctgcatgttcttcctaagcgtcatcttgagccttgcacatgatactcagattcctcacccttgcttaggagtaaaacataatacactttacagggtgatatctccatagttatttgaagtggcttggaaaaagcaagattaacttctgacattggataaaaatcaacaaatcagccctagagttattcaaatggtaattgacaaaaactaaaatatttcccttcgagaaggagtggaatgtggtttggcagaacaactgcatttcacagcttttccggttaaattggagcactaaacgtttagatgcataccaaattatgcatgggcccttaatataaaaggctggctaccagctttgacacagcactattcatcctctggccaaacaactgtggttaaacaacacatgtaaattgctttttaacagctgatactataataagacaaagccaaaatgcaaaaattgggctttgattggcactttttgaaaaatatgcaacaaatatgggatgtaatctggatggccgcttctgtacttaatgtgaagtatttagatacctttttgaacacttaacagtttcttctgacaatgacttttgtaaggattggtactatctatcattccttataatgtacattgtctgtcactaatcctcagatcttgctgtattgtcacctaaattggtacaggtactgatgaaaatatctaatggataatcataacactcttggtcacatgtttttcctgcagcctgaaggtttttaaaagaaaaagatatcaaatgcctgctgctaccacccttttaaattgctatcttttgaaaagcaccagtatgtgttttagattgatttccctattttagggaaatgacagacagtagtttcagttctgatggtataagcaaaacaaataaaacatgtttataaaagttgtatcttgaaacactggtgttcaacagctagcagcttatgtggttcaccccatgcattgttagtgtttcagattttatggttatctccagcagctgtttctgtagtacttgcatttatc

SEQ ID NO: 10 - protein, 702 aa, Canis lupus
  source 1..702: note = Canis familiaris
MPSATSLSGSGSKSSGPPPPSGSSGSEAAAAAGAAGAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKSVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTDLQQEQLQTVVGTYHGSQDQPHQVTGNHQQPPQQNTGFPRSSQPYYNSRGVSRGGSRGARGLMNGYRGPANGFRGGYDGYRPSFSNTPNSGYTQSQFSAPRDYSGYQRDGYQQNFKRGSGQSGPRGAPRGNILWW

SEQ ID NO: 11 - DNA, 3306 bp, Canis lupus
  source 1..3306: mol_type = unassigned DNA; note = Canis familiaris
  CDS 1..2040
atgccgtcggccaccagcctcagcggaagcggcagcaagtcgtcgggcccgccgcccccgtcgggttcctccgggagcgaggcggcggcggcggcgggggcggcgggggcggcgggggccggggcggctgcgcccgcctcccagcaccccgcgaccggcaccggcgctgtccagaccgaggccatgaagcagatcctcggggtgatcgacaagaaactccggaacctggagaagaaaaagggcaagcttgatgattaccaggaacgaatgaacaaaggggaaaggcttaatcaagatcagctggatgccgtatctaagtaccaggaagtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagaccttcagcaagaacagcttcaaacagtggttggcacttaccatggttcccaggaccagccccaccaagtgactggtaaccatcagcagcctccccagcagaacactggatttccacgtagcagtcagccctattacaatagtcgtggtgtgtctcgtggtggttcccgtggtgctagaggcttaatgaatggatacaggggccctgccaatggattcagaggaggatatgatggttaccgcccttcattctctaacactccaaacagtggttatacacagtctcagttcagtgctccccgggactactctggctatcagcggggatgccgcaaatgaacactcagcaagtgaattaatctgattcacaggattatgtttaaacgccaaaaacacactggccagtgtaccataatatgttaccagaagagttattatctatttgttctccctttcaggaaacttattgtaaagggactgttttcatcccataaagacaggactacaattgtcagctttatattacctggatatggaaggaaactatttttattctgcatgttcttcctaagcgtcatcttgagccttgcacatgatactcagattcctcacccttgcttaggagtaaaacataatacactttacagggtgatatctccatagttatttgaagtggcttggaaaaagcaagattaacttctgacattggataaaaatcaacaaatcagccctagagttattcaaatggtaattgacaaaaactaaaatatttcccttcgagaaggagtggaatgtggtttggcagaacaactgcatttcacagcttttccggttaaattggagcactaaacgtttagatgcataccaaattatgcatgggcccttaatataaaaggctggctaccagctttgacacagcactattcatcctctggccaaacaactgtggttaaacaacacatgtaaattgctttttaacagctgatactataataagacaaagccaaaatgcaaaaattgggctttgattggcactttttgaaaaatatgcaacaaatatgggatgtaatctggatggccgcttctgtacttaatgtgaagtatttagatacctttttgaacacttaacagtttcttctgacaatgacttttgtaaggattggtactatctatcattccttataatgtacattgtctgtcactaatcctcagatcttgctgtattgtcacctaaattggtacaggtactgatgaaaatatctaatggataatcataacactcttggtcacatgtttttcctgcagcctgaaggtttttaaaagaaaaagatatcaaatgcctgctgctaccacccttttaaattgctatcttttgaaaagcaccagtatgtgttttagattgatttccctattttagggaaatgacagacagtagtttcagttctgatggtataagcaaaacaaataaaacatgtttataaaagttgtatcttgaaacactggtgttcaacagctagcagcttatgtggttcaccccatgcattgttagtgtttcagattttatggttatctccagcagctgtttctgtagtacttgcatttatc

SEQ ID NO: 12 - protein, 679 aa, Canis lupus
  source 1..679: note = Canis familiaris
MPSATSLSGSGSKSSGPPPPSGSSGSEAAAAAGAAGAAGAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKSVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQADPLVRRQRVQDLMAQMQGPYNFIQDSMLDFENQTLDPAIVSAQPMNPTQNMDMPQLVCPPVHSESRLAQPNQVPVQPEATQVPLVSSTSEGYTASQPLYQPSHATEQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTDLQQEQLQTVVGTYHGSQDQPHQVTGNHQQPPQQNTGFPRSSQPYYNSRGVSRGGSRGARGLMNGYRGPANGFRGGYDGYRPSFSNTPNSGYTQSQFSAPRDYSGYQRGCRK

SEQ ID NO: 13 - DNA, 2281 bp, Canis lupus
  source 1..2281: mol_type = unassigned DNA; note = Canis familiaris
  CDS 1..2154
atgccgtcggccaccagcctcagcggaagcggcagcaagtcgtcgggcccgccgcccccgtcgggttcctccgggagcgaggcggcggcggcggcgggggcggcgggggcggcgggggccggggcggctgcgcccgcctcccagcaccccgcgaccggcaccggcgctgtccagaccgaggccatgaagcagatcctcggggtgatcgacaagaaactccggaacctggagaagaaaaagggcaagcttgatgattaccaggaacgaatgaacaaaggggaaaggcttaatcaagatcagctggatgccgtatctaagtaccaggaagtcacaaataacttggagtttgcaaaagaattacagaggagtttcatggcattaagtcaagatattcagaaaacaataaagaagactgcacgtcgggagcagcttatgagagaggaagcggaacaaaaacgtttaaaaactgtacttgagctccagtatgttttggacaaattgggagatgatgaagtgagaactgacctgaagcaaggtttgaatggagtgccaatattgtctgaagaagaattgtcgttgttggatgaattctacaaattagcagaccctgaacgggacatgagcttgaggttgaatgagcagtatgaacatgcttccattcacctgtgggacttgctggaaggaaaggaaaagtctgtatgtggaacaacctataaagcactaaaggaaattgttgagcgtgttttccagtcaaattactttgacagcactcacaaccaccagaatgggctatgtgaggaagaagaggcagcctcagcacctacagttgaagaccaggtagctgaagctgagcctgagccagcagaagaatacactgaacaaagtgaagttgaatcaacagagtatgtaaatagacaatttatggcagaaacacagttcagcagtggtgaaaaggagcaggtagatgagtggacggtcgaaacagtggaggtggtgaattcactccagcagcaacctcaggctgcgtctccttcagtaccagagccccactctttgactccggtggctcaggcagatccccttgtgagaagacagcgagtccaggaccttatggcgcagatgcaggggccctataatttcatacaggattcaatgctggattttgaaaaccagacactcgatcctgccattgtatctgcacagcctatgaatccgacacaaaacatggacatgccccagctggtttgccctccagttcattctgaatctagacttgctcaacctaatcaagttcctgtacaaccagaagctacacaggttcctttggtttcatccacaagtgaggggtatacagcatctcaacccttgtaccagccttctcatgctacagagcaacgaccacaaaaggaaccaattgaccagattcaggcaacaatctctttaaatacagaccagactacagcgtcatcatcccttccggctgcttctcagcctcaggtattccaggctgggacaagcaaaccattacatagcagtggaatcaatgtaaatgcagctccattccaatccatgcaaacggtgttcaatatgaatgccccagttcctcctgttaatgaaccagaaactttgaaacaacaaaatcagtaccaggccagttataaccagagcttttctagtcagcctcaccaagtagaacaaacagaccttcagcaagaacagcttcaaacagtggttggcacttaccatggttcccaggaccagccccaccaagtgactggtaaccatcagcagcctccccagcagaacactggatttccacgtagcagtcagccctattacaatagtcgtggtgtgtctcgtggtggttcccgtggtgctagaggcttaatgaatggatacaggggccctgccaatggattcagaggaggatatgatggttaccgcccttcattctctaacactccaaacagtggttatacacagtctcagttcagtgctccccgggactactctggctatcagcgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaaacgccaaaaacacactggccagtgtaccataatatgttaccagaagagttattatctatttggactgttttcatcccataaagacaggactacaattgtcagc

SEQ ID NO: 14 - protein, 717 aa, Canis lupus; identical to SEQ ID NO: 8
  source 1..717: note = Canis familiaris

SEQ ID NO: 15 - DNA, 3386 bp, Bos taurus
  source 1..3386: mol_type = unassigned DNA
  CDS 82..2208
cgcgtctcgccccgtccaccgattgactcgccgctcttgtccttcctcccgctctttcttctctccccttacggtttcaagatgccttcggccaccagccacagcggaagcggcagcaagtcgtccggaccgccaccgccgtcgggttcctccgggaatgaggcgggggccggggccgccgcgccggcttcccaacaccccatgaccggcaccggggctgtccagaccgaggccatgaagcagattctcggggtgatcgacaagaaacttcggaacctggagaagaaaaagggcaagcttgatgattatcaggaacgaatgaacaaaggggaaaggcttaatcaagatcagctggatgccgtgtctaagtaccaggaagtcacaaataacttggagtttgcaaaagaattacagaggagtttcatggcattaagccaagatattcagaaaacaataaagaagacagcacgtcgggagcagcttatgagagaggaagctgaacagaaacgtttaaaaacagtacttgagctgcagtatgttttggacaaactaggagatgatgaagtgagaactgacctgaagcaaggtttgaatggagtgccaatattgtctgaagaggagttgtcgttgttagatgagttctacaaattagcagaccctgaacgagacatgagcttgaggttgaatgagcagtatgaacatgcctccattcacctgtgggacttgctggaaggaaaggaaaaacctgtatgtggaacaacttataaagctctaaaggaaattgttgagcgtgttttccagtcaaactactttgacagcacccacaaccaccagaatggtctgtgtgaggaagaggaggcagcctcagcacctacagttgaagaccaggcagctgaagctgaacctgagccagtggaagaatatactgaacaaaatgaggttgaatcaacagagtatgtaaatagacaatttatggcagaaacacagttcagcagtggtgaaaaggagcaggtagatgattggacagttgaaacagttgaggtggtaaattcactccagcagcaacctcaggctgcatctccttcagtaccagaaccccactctttgaccccagtggctcaagccgatcccctcgtgagaagacagcgagtacaggaccttatggcacaaatgcaggggccctataatttcatacaggattcaatgttggattttgaaaaccagacacttgatcctgccattgtatctgcacagccgatgaatccagcacagaacatggacataccccagctggtttgccctccagttcattctgaatctagacttgctcaacctaatcaagtttctgtacagccagaagctacacaggttcctttggtttcatccacaagtgagggatatacagcatctcaacccttgtaccaaccttctcatgctactgaccaacgaccacaaaaggaaccgattgatcagattcaggcgacgatctctttaaatacagaccagactacagcatcatcatcccttcctgctgcttctcagcctcaagtgttccaggctgggacaagcaaacctttacatagcagtggaatcaatgtaaatgcagctccattccaatccatgcaaacggtattcaatatgaatgccccagttcctcctgttaatgaaccagaaactttaaaacagcaaaatcagtaccaggccagttacaaccagagcttttccagtcagcctcaccaagtagaacaaacagagcttcagcaagaacagcttcaaacagtggttggcacttatcatggttctcaggaccagccccatcaagtgactggtaaccaccagcagcctcctcagcagaacactggatttccacgtagcaatcagccctattacaacagtcgtggtgtgtctcgtggaggttcccgtggtgctagaggcttgatgaatggatacagaggacctgctaatggattcagaggaggatatgatggttaccgcccttcattctctactaacactccaaacagtggttatacacaatctcaattcagtgctccccgggactactctggctatcagcgggatggatatcagcagaatttcaagcgaggctctgggcagagtggaccacggggagccccacgaggtcgtggagggcccccaagacccaacagagggatgccgcaaatgaacactcagcaagtgaattaatctgattcacaggattatgtttaatcgccaaaaacacactggccagtgtaccataatatgttaccagaagagttattatctatttgttctccctttcaggaaacttattgtaaagggactgttttcatcccataaagacaggactacaattgtcagctttatattacctggatatggaaggaaactatttttactctgcatgttctgtcctaagcgtcatcttgagccttgcacatgatactcagattcctcacccttgcttaggagtaaaacataatatactttaatggggtgatatctccatagttatttgaagtggcttggataaagcaagactgacttctgacattggataaaatctacaaatcagccctagagtcattcagtggtaactgacaaaactaaaatatttcccttgaaaggaagatggaaggagtggagtgtggtttggcagaacaactgcatttcacagcttttccacttaaattggagcactgaacatttagatgcataccgaattatgcatgggccctaatcacacagacaaggctggtgccagccttaggcttgacacggcagtgttcaccctctggccagacgactgtggttcaagacacatgtaaattgctttttaacagctgatactgtataagacaaagccaaaatgcaaaattaggctttgattggcacttttcgaaaaatatgcaacaattaagggatataatctggatggccgcttctgtacttaatgtgaaatatttagatacctttcaaacacttaacagtttctttgacaatgagttttgtaaggattggtagtaaatatcattccttatgacgtacattgtctgtcactaatccttggatcttgctgtattgtcacctaaattggtacaggtactgatgaaaatctaatggataatcataacactcttggttacatgtttttcctgcagcctgaaagtttttataagaaaaagacatcaaatgcctgctgctgccacccttttaaattgctatcttttgaaaagcaccagtatgtgttttagattgatttccctattttagggaaatgacagtcagtagtttcacttctgatggtataagcaaacaaataaaacatgtttataaaaaaaaaaaaaaaaaaaaaaaaaaaaaaaaaaaaaaaaaaaaaaaaa

SEQ ID NO: 16 - protein, 708 aa, Bos taurus
MPSATSHSGSGSKSSGPPPPSGSSGNEAGAGAAAPASQHPMTGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDEVRTDLKQGLNGVPILSEEELSLLDEFYKLADPERDMSLRLNEQYEHASIHLWDLLEGKEKPVCGTTYKALKEIVERVFQSNYFDSTHNHQNGLCEEEEAASAPTVEDQAAEAEPEPVEEYTEQNEVESTEYVNRQFMAETQFSSGEKEQVDDWTVETVEVVNSLQQQPQAASPSVPEPHSLTPVAQADPLVRRQRVQDLMAQMQGPYNFIQDSMLDFENQTLDPAIVSAQPMNPAQNMDIPQLVCPPVHSESRLAQPNQVSVQPEATQVPLVSSTSEGYTASQPLYQPSHATDQRPQKEPIDQIQATISLNTDQTTASSSLPAASQPQVFQAGTSKPLHSSGINVNAAPFQSMQTVFNMNAPVPPVNEPETLKQQNQYQASYNQSFSSQPHQVEQTELQQEQLQTVVGTYHGSQDQPHQVTGNHQQPPQQNTGFPRSNQPYYNSRGVSRGGSRGARGLMNGYRGPANGFRGGYDGYRPSFSTNTPNSGYTQSQFSAPRDYSGYQRDGYQQNFKRGSGQSGPRGAPRGRGGPPRPNRGMPQMNTQQVN

SEQ ID NO: 17 - DNA, 3150 bp, Equus caballus
  source 1..3150: mol_type = unassigned DNA
  CDS 1..1917
atggagggcaagctcgatgattaccaagagcgaatgaacaaaggagaaaggcttaatcaggatcagctggatgctgtgtctaagtaccaggaagtcacaaataacttggagtttgcgaaagaattgcagaggagtttcatggcgttgagtcaggatattcagaaaacaataaagaagacggcacgtcgggagcagcttatgagagaagaagctgaacagaaacgtttaaaaactgtacttgagctgcagtatgttttggacaaattgggagatgaagaagtgcgaactgacctgaaacaaggtttgaatggagtgccaatactctctgaagaagagttgtcgctgttggatgagttctacaagttagcagaccctgtacgggacatgagcttgaggttgaatgagcagtatgagcatgcctccattcacctgtgggacttgctggaagggaaggaaaaatctgtctgtggaacaacctataaagctctgagggaaattgttgagcgtgttttccagtccaactactttgacagcacccacaaccaccagaatgggctctgtgaggaggaagaggctacctcagctccaacagctgaagaccagggagctgaagctgaacctgagccagcagaagaatacactgaacaaagtgaagttgaatcaacagagtatgtaaatagacagtttatggcagaagcgcagttcagtggtgagaaggagcaggtggatgagtggacagtcgagacggtcgaggtggtaaattcactccagcagcaacctcaggctgcatctccttcagtaccggagccccactctttgactccagtggctcaggcagatccccttgtgagaagacagcgagtacaggaccttatggcgcaaatgcaggggccctataatttcatacaggattcaatgctggattttgaaaaccagacacttgatcctgccattgtatctgcacagcctatgaatccagcacagaatatggacatgccccagctggtttgccctccagttcatgctgaatctagacttgctcaacctaatcaagttcctgtacaaccagaagctacacaggttcctttggtttcatccacaagtgaggggtatacagcatctcagcccttgtaccagccttctcatgctacagagcaacgaccgcaaaaggaaccgactgaccagatccaggcaacaatctctttaaatacagaccagactacagcatcatcatcccttcctgctgcttctcagcctcaggtgttccaggctgggacaagcaaacctttacacagcagtgggatcaatgtaaatgcagcgccattccagtccatgcaaacggtgttcaacatgaatgccccggttcctcctgttaatgaaccagaaactttaaaacagcaaaatcagtaccaggccagctataaccagagcttttccagtccgcctcaccaagtagagcagacagagcttccgcaagagcagcttcagacggtggttggtacttaccatgcttcccaagaccagccccatcaagtgaccggtaaccaccagcagcctccccagcagaacactgggtttccacgtagcagtcagccctattacaacagtcgtggtgtgtctcgtggaggctcccgtggtgctagaggcttgatgaatggatacaggggccctgccaatggattcagaggaggatatgatggttaccgcccttcgttctctaacactccaaacagcggttacacacagtctcagttcagtgctccccgggactactctggctatcagcgggatggatatcagcagaatttcaagcgaggctctgggcagagtggaccccggggagccccacgaggtcgtggagggcccccaagacccaacagagggatgccgcaaatgaacactcagcaagtgaattaatctgattcacaggattatctttaatcgccaaaacacactggccagtgtaccataatatgttaccagaagagttattatctatttgttctccctttcaggaaacttattgtaaagggactgttttcatcccataaagacaggactacagttgtcagctttatattacctggatatggaaggaaactatttttactctgcatgttctgtcctaagcgtcatcttgagccttgcacatgatactcagattcctttcccttgcttaggagtaaaacataatatactttatggggtgataatatctccatagttatttgaagtggcttggaaaaagcaagattgacttttgacattggataaaatctacaaatcagccctagagtttcatggtcattcacaaaactaaaatatttcccttgaaaggaagatggaaggactggagtgtggtttggcagaacaactgcatttcacagcttttcctattaaattggagcactgaatgttaaatgcataccaaattatgcatgggcccttaatcacacatacatggctaccagctttgacacagcactattcatcctctggccaaacgactgtggttaaaaacacgtgtaaattgctttttaacagctgatactgtaaaagacaaagctaaaatgcaaaattaggctttcattggcacttttcgaaaaatatgcaacaaatttgggatgtaatctggatggccacttctgtacttaatgtgaagtatttagatacctttttgaacacttaacagtttcttcgacaatgacttttgtaaggattggtagtatatatcattccttatgacatacattgtctgttgctaatccttggatcttgctgtattgtcacctaaattggtacaggtactgatgaaaatctctcatggataaacctaacactcttcgtcacatgtttttcctgcagcctgaaggtttttaaaaggaaaagatatcaaatgcctgctgctaccacccttttaaattgctatcttttgaaaagcaccagtatgtgtttttagattgatttccctattttagggaaatgacagtcagtagtttcagttctgatggtataagcaaagcaaataaaacgtgtttataaaagttgtatcttgaaacactggtgttcaacagctagcagcttctgtggttcaccccctgccttgttagtgttacccatttatggttatctccagcagcaatttctcta

SEQ ID NO: 18 - protein, 638 aa, Equus caballus
MEGKLDDYQERMNKGERLNQDQLDAVSKYQEVTNNLEFAKELQRSFMALSQDIQKTIKKTARREQLMREEAEQKRLKTVLELQYVLDKLGDEEVRTDLKQGLNGVPILSEEELSLLDEFYKLADPVRDMSLRLNEQYEHASIHLWDLLEGKEKSVCGTTYKALREIVERVFQSNYFDSTHNHQNGLCEEEEATSAPTAEDQGAEAEPEPAEEYTEQSEVESTEYVNRQFMAEAQFSGEKEQVDEWTVETVEVVNSLQQQPQAASPSVPEPHSLTPVAQADPLVRRQRVQDLMAQMQGPYNFIQDSMLDFENQTLDPAIVSAQPMNPAQNMDMPQLVCPPVHAESRLAQPNQVPVQPEATQVPLVSSTSEGYTASQPLYQPSHATEQRPQKEPTDQIQATISLNTDQTTASSSLPAASQPQVFQAGTSKPLHSSGINVNAAPFQSMQTVFNMNAPVPPVNEPETLKQQNQYQASYNQSFSSPPHQVEQTELPQEQLQTVVGTYHASQDQPHQVTGNHQQPPQQNTGFPRSSQPYYNSRGVSRGGSRGARGLMNGYRGPANGFRGGYDGYRPSFSNTPNSGYTQSQFSAPRDYSGYQRDGYQQNFKRGSGQSGPRGAPRGRGGPPRPNRGMPQMNTQQVN

SEQ ID NO: 19 - DNA, 6181 bp, Mus musculus
  source 1..6181: mol_type = unassigned DNA
  CDS 179..2302
gctggctggctaagtccctcccgcgccggctcttgtcccactaggagcagctcagagccgcggggacagggcgaagcggcctgcgcccacggagcgcacgtctctgttctcaacgcagcaccacccttgcccccctcggctgcccactccagacgtccagcggctccgcgcgcgcacgatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacaggattcaatgttggattttgaaaatcagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtcgtggagggcccccaagacccaacagagggatgccgcaaatgaacactcagcaagtgaattaatgtgatacacaggattatgtttaatcgccaaaaacacactggccagtgtaccataatatgttaccagaagagttattatctatttgttctccctttcaggaaacttattgtaaagggactgttttcatcccataaagacaggactgcaattgtcagctttacattacctggatatggaaggaaactatttttattctgcatgttctgtcctaagcgtcatcttgagccttgcacacaatacaatactcagattcctcacccttgcttaggagtaaaacattatatacttatggggtgataatatctccatagttagttgaagtggcttggaaaaaaaatgcaagattgaatttttgaccttggataaaatctacaatcagccctagaactattcagtggtaattgacaaagttaaagcattttctttgaaaggaagatggaaggagtggagtgtggtttagcaaaactgcatttcatagctttcccattaaattggagcaccgacagattaaaagcataccaaattatgcatgggtccttactcacacaagtgaggctggctaccagccttgacatagcactcactagtcttctggccaaacgactgtgattaaaacacatgtaaattgctctttagtagtggatactgtgtaagacaaagccaaattgcaaatcaggctttgattggctcttctggaaaatatgcatcaaatatgggggataatctggatgggctgctgctgtgctcaatgtgaactatttagatacctttggaacacttaacagtttctctgaacaatgacttacatggggattggtcctgtttgtcattcctcaccataattgcattgtcatcactaatccttggatcttgctgtattgttactcaaattggtaataggtactgatggaaatcgctaatggatggataatcataacacttttggtcacatgttttctcctgcagcctgaaagttcttaaagaaaaagatatcaaatgcctgctgctaccacccttttaaattgctatctttagaaaagcaccggtatgtgttttagattcatttccctgttttagggaaatgacaggcagtagtttcagttctgatggcaaaacaaataaaaacatgtttctaaaagttgtatcttgaaacactggtgttcaacagctagcagctaaagtaattcaacccatgcattgctagtgtcacagcctttggttatgtctagtagctgtttctgaagtattttcatttatcttttgtcaaatttaaccctgtttgaattctctcctttcctcaaggagacacttatgttcaaagtgttgattctttgccttaggtgcatagagagtagacagtttggagatggaaaggttagcagtgacttagccatatgttctgtgttggaatttgtgctagcagtttgagcactagctctgcgtgcctatgaactgaatgctgcttgtcccattccattttatgtcatggagaaataattccacttggtaacacaaaggctaagttaatgttattttctgtacagaaattaaattttacttttagccttttgtaaactttttttttttttttccaagccggtatcagctactcaaaacaattctcagatattcatcattagacaactggagtttttgctggttttgtagcctactaaaactgctgaggctgttgaacattccacattcaaaagttttgtagggtggtggataatggggaagcttcaatgtttattttaaaataaataaaataagttcttgacttttctcatgtgtggttatggtacatcatattggaagggttatctgtttacttttgccaagactattttgccagcacctacacttgtgtgctttaaaagacaactacctgggatgtaccacaaccatatgttaattgtattttattgggatggataaaatgtttgtggtttattggataatccctagatggtgtgttacgtgtgtagaatataattttatgatagtaagaaagcaaaattgaagaaaataagtttagtattgaatttgagttctgaagtgaattcagggaatgtctcacgtttcgggcttctacccaaagtgtagggcagaaggtgtaaaagttgtttgtagtttgacttgtttattttttaagttgcttattcctttcaacagcaacatatcattagctgtcattctaccattgcagttctagtgagttttaacgtctgcattcaagactgttttaaaagcaacctcactggacagagaactgctaaagtcttttccttaagatctgagtctttgttactcagtatcttctataatatgcaaatgcttgtctagaggcagaagaccttttgtttggtcaagtgtgtattttaccagagtacagggaactgatggtcctacatgtctcttagtgtagtaagactataaaatcttttgtacatgcacaattcacagtatgtttagataccacgtgtataatgcccccccctcccccaggtagcatgccattgatgactttttgcttagggccattttattaccagggccttaatattcctaaaaagatgattttttttcatcctttctcctcttttgatcattgtatcttgatattaaaaacatgaccttccaatgattgtagtaaattaacttctatagttcttttgtctctatatgtattcatatatatgctattgtatagagacttcaaggagacatggagatgcatgcttattctcaggttcattcactaaggtgcttggcagacaaccagtttctaagtgcagaatgtagttaagcagcttcatatatgtgccaggcaatttgttttgttaaattttcatctacttaaggaaatagggtattgtagcttaggctgatcatacccttcatttcaaccttaagctctcaacctgcatccatccgacttgagctattaagtactttagttttatcgagtataagttaacagaaaaagtaaattaagctttgcctttactattttgaatttatatacattctggaaaaacttagaaactgttgtatatttcattagattaaattatatgaaaatgtgattgtttatagcaaagcctgtgagttgcatacaccctaaggaaaactccttaagtgctccttgaagagagaagaaacaattctgggtctggtctttttaagaacaaagctagactactgtatgttagcactgtacattaatagtctgttgtgaagcttgagcagtttcctgcatagccttgatccttcaccgttggcattgaaaatagcagtatccctgatgtacttaaaacttaaagtcaggttttggtatatttatttgtaagtcttaatttcctctaaatactatatctctttagcgagacaacctgaaatttattagcacatttgggtatctcttgcttggcattatggccagtgttaactattcagtggtgaaaaaattacccctcaagacactggagtgaccccagatgtgtgtagtaagtggcatggttcaactgtgtggttaatgataaatatatgacttagtcggtatgatctggaaagacttgattgaaagataattcagctgacataaggatgagtgaggagtggcaaactggataaaagagtcaagagacctgtattccagtgactcctgttttgtttaagcattagcaagatctgtctggggaaactggatagggcagttttcttccatgtttagtttttgtctcaacatttggaagctattgaaggttttaaaatggtgtgtattgtttttttttggggggggggtggccagaatagtgggtcatctaataaaactgccatttaaaagatcaaaaaaaaaaaaaaaaaaaaaaaaa

SEQ ID NO: 20 - protein, 707 aa, Mus musculus
MPSATSHSGSGSKSSGPPPPSGSSGSEAAAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDDVRTDLKQGLSGVPILSEEELSLLDEFYKLVDPERDMSLRLNEQYEHASIHLWDLLEGKEKPVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQSDPLVRRQRVQDLMAQMQGPYNFIQDSMLDFENQTLDPAIVSAQPMNPTQNMDMPQLVCPQVHSESRLAQSNQVPVQPEATQVPLVSSTSEGYTASQPLYQPSHATEQRPQKEPMDQIQATISLNTDQTTASSSLPAASQPQVFQAGTSKPLHSSGINVNAAPFQSMQTVFNMNAPVPPANEPETLKQQSQYQATYNQSFSSQPHQVEQTELQQDQLQTVVGTYHGSQDQPHQVPGNHQQPPQQNTGFPRSSQPYYNSRGVSRGGSRGARGLMNGYRGPANGFRGGYDGYRPSFSNTPNSGYSQSQFTAPRDYSGYQRDGYQQNFKRGSGQSGPRGAPRGRGGPPRPNRGMPQMNTQQVN

SEQ ID NO: 21 - DNA, 6141 bp, Mus musculus
  source 1..6141: mol_type = unassigned DNA
  CDS 139..2262
cccaccgcgcgcgcgcgtagccgcctgcccgcccgcccgctgcgcgttttgtcccgcgtctctccccgtccgtctcctgacttgctggtcttgtccttccctcccgcttttttcctctcctctcttctcggtctaaagatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacaggattcaatgttggattttgaaaatcagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtcgtggagggcccccaagacccaacagagggatgccgcaaatgaacactcagcaagtgaattaatgtgatacacaggattatgtttaatcgccaaaaacacactggccagtgtaccataatatgttaccagaagagttattatctatttgttctccctttcaggaaacttattgtaaagggactgttttcatcccataaagacaggactgcaattgtcagctttacattacctggatatggaaggaaactatttttattctgcatgttctgtcctaagcgtcatcttgagccttgcacacaatacaatactcagattcctcacccttgcttaggagtaaaacattatatacttatggggtgataatatctccatagttagttgaagtggcttggaaaaaaaatgcaagattgaatttttgaccttggataaaatctacaatcagccctagaactattcagtggtaattgacaaagttaaagcattttctttgaaaggaagatggaaggagtggagtgtggtttagcaaaactgcatttcatagctttcccattaaattggagcaccgacagattaaaagcataccaaattatgcatgggtccttactcacacaagtgaggctggctaccagccttgacatagcactcactagtcttctggccaaacgactgtgattaaaacacatgtaaattgctctttagtagtggatactgtgtaagacaaagccaaattgcaaatcaggctttgattggctcttctggaaaatatgcatcaaatatgggggataatctggatgggctgctgctgtgctcaatgtgaactatttagatacctttggaacacttaacagtttctctgaacaatgacttacatggggattggtcctgtttgtcattcctcaccataattgcattgtcatcactaatccttggatcttgctgtattgttactcaaattggtaataggtactgatggaaatcgctaatggatggataatcataacacttttggtcacatgttttctcctgcagcctgaaagttcttaaagaaaaagatatcaaatgcctgctgctaccacccttttaaattgctatctttagaaaagcaccggtatgtgttttagattcatttccctgttttagggaaatgacaggcagtagtttcagttctgatggcaaaacaaataaaaacatgtttctaaaagttgtatcttgaaacactggtgttcaacagctagcagctaaagtaattcaacccatgcattgctagtgtcacagcctttggttatgtctagtagctgtttctgaagtattttcatttatcttttgtcaaatttaaccctgtttgaattctctcctttcctcaaggagacacttatgttcaaagtgttgattctttgccttaggtgcatagagagtagacagtttggagatggaaaggttagcagtgacttagccatatgttctgtgttggaatttgtgctagcagtttgagcactagctctgcgtgcctatgaactgaatgctgcttgtcccattccattttatgtcatggagaaataattccacttggtaacacaaaggctaagttaatgttattttctgtacagaaattaaattttacttttagccttttgtaaactttttttttttttttccaagccggtatcagctactcaaaacaattctcagatattcatcattagacaactggagtttttgctggttttgtagcctactaaaactgctgaggctgttgaacattccacattcaaaagttttgtagggtggtggataatggggaagcttcaatgtttattttaaaataaataaaataagttcttgacttttctcatgtgtggttatggtacatcatattggaagggttatctgtttacttttgccaagactattttgccagcacctacacttgtgtgctttaaaagacaactacctgggatgtaccacaaccatatgttaattgtattttattgggatggataaaatgtttgtggtttattggataatccctagatggtgtgttacgtgtgtagaatataattttatgatagtaagaaagcaaaattgaagaaaataagtttagtattgaatttgagttctgaagtgaattcagggaatgtctcacgtttcgggcttctacccaaagtgtagggcagaaggtgtaaaagttgtttgtagtttgacttgtttattttttaagttgcttattcctttcaacagcaacatatcattagctgtcattctaccattgcagttctagtgagttttaacgtctgcattcaagactgttttaaaagcaacctcactggacagagaactgctaaagtcttttccttaagatctgagtctttgttactcagtatcttctataatatgcaaatgcttgtctagaggcagaagaccttttgtttggtcaagtgtgtattttaccagagtacagggaactgatggtcctacatgtctcttagtgtagtaagactataaaatcttttgtacatgcacaattcacagtatgtttagataccacgtgtataatgcccccccctcccccaggtagcatgccattgatgactttttgcttagggccattttattaccagggccttaatattcctaaaaagatgattttttttcatcctttctcctcttttgatcattgtatcttgatattaaaaacatgaccttccaatgattgtagtaaattaacttctatagttcttttgtctctatatgtattcatatatatgctattgtatagagacttcaaggagacatggagatgcatgcttattctcaggttcattcactaaggtgcttggcagacaaccagtttctaagtgcagaatgtagttaagcagcttcatatatgtgccaggcaatttgttttgttaaattttcatctacttaaggaaatagggtattgtagcttaggctgatcatacccttcatttcaaccttaagctctcaacctgcatccatccgacttgagctattaagtactttagttttatcgagtataagttaacagaaaaagtaaattaagctttgcctttactattttgaatttatatacattctggaaaaacttagaaactgttgtatatttcattagattaaattatatgaaaatgtgattgtttatagcaaagcctgtgagttgcatacaccctaaggaaaactccttaagtgctccttgaagagagaagaaacaattctgggtctggtctttttaagaacaaagctagactactgtatgttagcactgtacattaatagtctgttgtgaagcttgagcagtttcctgcatagccttgatccttcaccgttggcattgaaaatagcagtatccctgatgtacttaaaacttaaagtcaggttttggtatatttatttgtaagtcttaatttcctctaaatactatatctctttagcgagacaacctgaaatttattagcacatttgggtatctcttgcttggcattatggccagtgttaactattcagtggtgaaaaaattacccctcaagacactggagtgaccccagatgtgtgtagtaagtggcatggttcaactgtgtggttaatgataaatatatgacttagtcggtatgatctggaaagacttgattgaaagataattcagctgacataaggatgagtgaggagtggcaaactggataaaagagtcaagagacctgtattccagtgactcctgttttgtttaagcattagcaagatctgtctggggaaactggatagggcagttttcttccatgtttagtttttgtctcaacatttggaagctattgaaggttttaaaatggtgtgtattgtttttttttggggggggggtggccagaatagtgggtcatctaataaaactgccatttaaaagatcaaaaaaaaaaaaaaaaaaaaaaaaa

SEQ ID NO: 22 - protein, 707 aa, Mus musculus; identical to SEQ ID NO: 20

SEQ ID NO: 23 - DNA, 6114 bp, Mus musculus
  source 1..6114: mol_type = unassigned DNA
  CDS 139..2235
cccaccgcgcgcgcgcgtagccgcctgcccgcccgcccgctgcgcgttttgtcccgcgtctctccccgtccgtctcctgacttgctggtcttgtccttccctcccgcttttttcctctcctctcttctcggtctaaagatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtcgtggagggcccccaagacccaacagagggatgccgcaaatgaacactcagcaagtgaattaatgtgatacacaggattatgtttaatcgccaaaaacacactggccagtgtaccataatatgttaccagaagagttattatctatttgttctccctttcaggaaacttattgtaaagggactgttttcatcccataaagacaggactgcaattgtcagctttacattacctggatatggaaggaaactatttttattctgcatgttctgtcctaagcgtcatcttgagccttgcacacaatacaatactcagattcctcacccttgcttaggagtaaaacattatatacttatggggtgataatatctccatagttagttgaagtggcttggaaaaaaaatgcaagattgaatttttgaccttggataaaatctacaatcagccctagaactattcagtggtaattgacaaagttaaagcattttctttgaaaggaagatggaaggagtggagtgtggtttagcaaaactgcatttcatagctttcccattaaattggagcaccgacagattaaaagcataccaaattatgcatgggtccttactcacacaagtgaggctggctaccagccttgacatagcactcactagtcttctggccaaacgactgtgattaaaacacatgtaaattgctctttagtagtggatactgtgtaagacaaagccaaattgcaaatcaggctttgattggctcttctggaaaatatgcatcaaatatgggggataatctggatgggctgctgctgtgctcaatgtgaactatttagatacctttggaacacttaacagtttctctgaacaatgacttacatggggattggtcctgtttgtcattcctcaccataattgcattgtcatcactaatccttggatcttgctgtattgttactcaaattggtaataggtactgatggaaatcgctaatggatggataatcataacacttttggtcacatgttttctcctgcagcctgaaagttcttaaagaaaaagatatcaaatgcctgctgctaccacccttttaaattgctatctttagaaaagcaccggtatgtgttttagattcatttccctgttttagggaaatgacaggcagtagtttcagttctgatggcaaaacaaataaaaacatgtttctaaaagttgtatcttgaaacactggtgttcaacagctagcagctaaagtaattcaacccatgcattgctagtgtcacagcctttggttatgtctagtagctgtttctgaagtattttcatttatcttttgtcaaatttaaccctgtttgaattctctcctttcctcaaggagacacttatgttcaaagtgttgattctttgccttaggtgcatagagagtagacagtttggagatggaaaggttagcagtgacttagccatatgttctgtgttggaatttgtgctagcagtttgagcactagctctgcgtgcctatgaactgaatgctgcttgtcccattccattttatgtcatggagaaataattccacttggtaacacaaaggctaagttaatgttattttctgtacagaaattaaattttacttttagccttttgtaaactttttttttttttttccaagccggtatcagctactcaaaacaattctcagatattcatcattagacaactggagtttttgctggttttgtagcctactaaaactgctgaggctgttgaacattccacattcaaaagttttgtagggtggtggataatggggaagcttcaatgtttattttaaaataaataaaataagttcttgacttttctcatgtgtggttatggtacatcatattggaagggttatctgtttacttttgccaagactattttgccagcacctacacttgtgtgctttaaaagacaactacctgggatgtaccacaaccatatgttaattgtattttattgggatggataaaatgtttgtggtttattggataatccctagatggtgtgttacgtgtgtagaatataattttatgatagtaagaaagcaaaattgaagaaaataagtttagtattgaatttgagttctgaagtgaattcagggaatgtctcacgtttcgggcttctacccaaagtgtagggcagaaggtgtaaaagttgtttgtagtttgacttgtttattttttaagttgcttattcctttcaacagcaacatatcattagctgtcattctaccattgcagttctagtgagttttaacgtctgcattcaagactgttttaaaagcaacctcactggacagagaactgctaaagtcttttccttaagatctgagtctttgttactcagtatcttctataatatgcaaatgcttgtctagaggcagaagaccttttgtttggtcaagtgtgtattttaccagagtacagggaactgatggtcctacatgtctcttagtgtagtaagactataaaatcttttgtacatgcacaattcacagtatgtttagataccacgtgtataatgcccccccctcccccaggtagcatgccattgatgactttttgcttagggccattttattaccagggccttaatattcctaaaaagatgattttttttcatcctttctcctcttttgatcattgtatcttgatattaaaaacatgaccttccaatgattgtagtaaattaacttctatagttcttttgtctctatatgtattcatatatatgctattgtatagagacttcaaggagacatggagatgcatgcttattctcaggttcattcactaaggtgcttggcagacaaccagtttctaagtgcagaatgtagttaagcagcttcatatatgtgccaggcaatttgttttgttaaattttcatctacttaaggaaatagggtattgtagcttaggctgatcatacccttcatttcaaccttaagctctcaacctgcatccatccgacttgagctattaagtactttagttttatcgagtataagttaacagaaaaagtaaattaagctttgcctttactattttgaatttatatacattctggaaaaacttagaaactgttgtatatttcattagattaaattatatgaaaatgtgattgtttatagcaaagcctgtgagttgcatacaccctaaggaaaactccttaagtgctccttgaagagagaagaaacaattctgggtctggtctttttaagaacaaagctagactactgtatgttagcactgtacattaatagtctgttgtgaagcttgagcagtttcctgcatagccttgatccttcaccgttggcattgaaaatagcagtatccctgatgtacttaaaacttaaagtcaggttttggtatatttatttgtaagtcttaatttcctctaaatactatatctctttagcgagacaacctgaaatttattagcacatttgggtatctcttgcttggcattatggccagtgttaactattcagtggtgaaaaaattacccctcaagacactggagtgaccccagatgtgtgtagtaagtggcatggttcaactgtgtggttaatgataaatatatgacttagtcggtatgatctggaaagacttgattgaaagataattcagctgacataaggatgagtgaggagtggcaaactggataaaagagtcaagagacctgtattccagtgactcctgttttgtttaagcattagcaagatctgtctggggaaactggatagggcagttttcttccatgtttagtttttgtctcaacatttggaagctattgaaggttttaaaatggtgtgtattgtttttttttggggggggggtggccagaatagtgggtcatctaataaaactgccatttaaaagatcaaaaaaaaaaaaaaaaaaaaaaaaa

SEQ ID NO: 24 - protein, 698 aa, Mus musculus
MPSATSHSGSGSKSSGPPPPSGSSGSEAAAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDDVRTDLKQGLSGVPILSEEELSLLDEFYKLVDPERDMSLRLNEQYEHASIHLWDLLEGKEKPVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQSDPLVRRQRVQDLMAQMQGPYNFIQTLDPAIVSAQPMNPTQNMDMPQLVCPQVHSESRLAQSNQVPVQPEATQVPLVSSTSEGYTASQPLYQPSHATEQRPQKEPMDQIQATISLNTDQTTASSSLPAASQPQVFQAGTSKPLHSSGINVNAAPFQSMQTVFNMNAPVPPANEPETLKQQSQYQATYNQSFSSQPHQVEQTELQQDQLQTVVGTYHGSQDQPHQVPGNHQQPPQQNTGFPRSSQPYYNSRGVSRGGSRGARGLMNGYRGPANGFRGGYDGYRPSFSNTPNSGYSQSQFTAPRDYSGYQRDGYQQNFKRGSGQSGPRGAPRGRGGPPRPNRGMPQMNTQQVN

SEQ ID NO: 25 - DNA, 3548 bp, Mus musculus
  source 1..3548: mol_type = unassigned DNA
  CDS 179..2257
gctggctggctaagtccctcccgcgccggctcttgtcccactaggagcagctcagagccgcggggacagggcgaagcggcctgcgcccacggagcgcacgtctctgttctcaacgcagcaccacccttgcccccctcggctgcccactccagacgtccagcggctccgcgcgcgcacgatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacaggattcaatgttggattttgaaaatcagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtaatatattgtggtggtgatcctagctcctatgtggagcttctgttctggccttggaagaactgttcatagtccgcatgtaggttacatgttaggaatacatttatcttttccagacttgttgctaaagattaaatgaaatgctctgtttctaaaatttcatcttgaatccaaattttaatttttgaatgactttccctgctgttgtcttcaaaatcagaacattttctctgcctcagaaaagcgtttttccaactggaaatttatttttcaggtcttaaaacctgctaaatgtttttaggaagtacctactgaaactttttgtaagacatttttggaacgagcttgaacatttatataaatttattaccctctttgatttttgaaacatgcatattatatttaggctgagaagcccttcaaatggccagataagccacagttttagctagagaaccatttagaattgacataactaatctaaacttgaacacttttaggaccaatgttagtgttctaaataccaacatatttctgatgtttaaacagatctcccaaattcttaggaccttgatgtcattaaaatttagaatgacaagcttaagaggctttagtttcatttgtttttcaagtaatgaaaaataatttcttacatgggcagatagttaatttgttgaacaattacaggtagcatttcatgtaatctgatgttctaaatggttctcttattgaaggaggttaaagaattaggtttcttacagtttttggctggccatgacatgtataaaatgtatattaaggaggaattataaagtactttaatttgaatgctagtggcaattgatcattaagaaagtactttaaagcaaaaggttaatgggtcatctgggaaaaatactgaagtatcaaaggtatttgcatgtgaatgtgggttatgttcttctatcccaccttgtagcatattctatgaaagttgagttaaatgatagctaaaatatctgtttcaacagcatgtaaaaagttattttaactgttacaagtcattatacaattttgaatgttctgtagtttctttttaacagtttaggtacaaaggtctgttttcattctggtgctttttattaattttgatagtatgatgtcacttcctattgaaatgtaagctagcgtgtaccttagaatgtgagctccatgagagcaggtaccttgtttgtcttcactgctgtatctattcccaacgcctcatgacagtgcctggcacatagtaggcactcaataaatacttgttgaatgaatgaaaaaaaaaaaaaa

SEQ ID NO: 26 - protein, 692 aa, Mus musculus
MPSATSHSGSGSKSSGPPPPSGSSGSEAAAGAAAPASQHPATGTGAVQTEAMKQILGVIDKKLRNLEKKKGKLDDYQERMNKGERLNQDQLDAVSKYQEVTNNLEFAKELQRSFMALSQDIQKTIKKTARREQLMREEAEQKRLKTVLELQYVLDKLGDDDVRTDLKQGLSGVPILSEEELSLLDEFYKLVDPERDMSLRLNEQYEHASIHLWDLLEGKEKPVCGTTYKALKEIVERVFQSNYFDSTHNHQNGLCEEEEAASAPTVEDQVAEAEPEPAEEYTEQSEVESTEYVNRQFMAETQFSSGEKEQVDEWTVETVEVVNSLQQQPQAASPSVPEPHSLTPVAQSDPLVRRQRVQDLMAQMQGPYNFIQDSMLDFENQTLDPAIVSAQPMNPTQNMDMPQLVCPQVHSESRLAQSNQVPVQPEATQVPLVSSTSEGYTASQPLYQPSHATEQRPQKEPMDQIQATISLNTDQTTASSSLPAASQPQVFQAGTSKPLHSSGINVNAAPFQSMQTVFNMNAPVPPANEPETLKQQSQYQATYNQSFSSQPHQVEQTELQQDQLQTVVGTYHGSQDQPHQVPGNHQQPPQQNTGFPRSSQPYYNSRGVSRGGSRGARGLMNGYRGPANGFRGGYDGYRPSFSNTPNSGYSQSQFTAPRDYSGYQRDGYQQNFKRGSGQSGPRGAPRGNILWW

SEQ ID NO: 27 - DNA, 3508 bp, Mus musculus
  source 1..3508: mol_type = unassigned DNA
  CDS 139..2217
cccaccgcgcgcgcgcgtagccgcctgcccgcccgcccgctgcgcgttttgtcccgcgtctctccccgtccgtctcctgacttgctggtcttgtccttccctcccgcttttttcctctcctctcttctcggtctaaagatgccctcggccaccagccacagcggaagcggcagcaaatcgtcgggaccgccgccgccgtccggttcctccgggagtgaggcggcggccggggcagctgcgccggcttctcagcatccggcaaccggcaccggcgccgtccagaccgaggccatgaagcagattctcggcgtaatcgacaagaaacttcggaacctggagaagaaaaagggtaaacttgatgattaccaggaacgaatgaataaaggggaaaggctcaatcaagaccagctggatgccgtatctaagtaccaggaagtcacaaataatttggagtttgcaaaggaattacagaggagtttcatggcattaagtcaagatattcagaaaacaataaagaagacagcacgtcgggaacagcttatgagagaagaagcagaacagaagcgcttaaaaactgtacttgagttacagtatgtattggataagctgggagatgatgatgtgagaacagatctgaaacaaggtttgagtggagtgccaatattgtctgaggaggagttgtcattgctggatgagttctacaagctcgtagatcctgagcgtgacatgagtttaaggttaaatgagcagtatgaacatgcctcaattcacttgtgggatttgctggaagggaaagaaaagcctgtgtgtggaacaacctataaagctctaaaggaaattgttgagcgtgttttccagtcaaactactttgatagcactcacaatcatcaaaatgggttgtgtgaggaggaagaggcggcttcagcgcccacagtggaggaccaggtagctgaagctgaacctgagccagcggaagaatacacagagcaaagtgaggttgaatcaacagagtatgtcaataggcagttcatggcagaaacacagttcagcagtggtgagaaggagcaagtggatgagtggacagttgaaacagttgaggttgtaaactcactccagcagcaacctcaggctgcgtccccttcagtcccagagccccactctttgactccagtggctcagtcagatccacttgtgagaaggcagcgtgtacaagatcttatggcacaaatgcaagggccctataatttcatacaggattcaatgttggattttgaaaatcagacgcttgatcctgccattgtatccgcacagcctatgaaccctacccagaacatggatatgcctcagctggtttgccctcaggttcattctgaatctagacttgcccaatctaatcaagttcctgtacaaccagaagccacacaggttcctttggtttcatccacaagtgaggggtatacagcatctcagcccttgtaccagccatctcatgctacggagcagcggccgcagaaagagccaatggatcagattcaggcaacaatatctttgaatacagaccagactacagcatcctcatcccttcctgctgcttctcagcctcaagtgttccaggctgggacaagtaaacctttgcacagcagtggaatcaatgtaaatgcagctccattccagtccatgcaaacggtgttcaatatgaatgctccagtccctcctgctaatgaaccagaaacgttaaaacaacagagtcagtaccaggccacttataaccagagtttttccagtcagcctcaccaagtggaacaaacagagcttcaacaagaccaactgcaaacggtggttggcacttaccatggatcccaggaccagcctcatcaagtgcctggtaaccaccagcaacccccacagcagaacactggctttccacgtagcagtcagccttattacaacagtcgtggggtatctcgaggagggtctcgtggtgccagaggcttgatgaatggatacaggggccctgccaatggatttagaggaggatatgatggttaccgcccttcattctcgaacactccaaacagtggttattcacagtctcagttcactgctccccgggactactctggttaccagcgggatggatatcagcagaatttcaagcgaggctctgggcagagtggaccacggggagccccacgaggtaatatattgtggtggtgatcctagctcctatgtggagcttctgttctggccttggaagaactgttcatagtccgcatgtaggttacatgttaggaatacatttatcttttccagacttgttgctaaagattaaatgaaatgctctgtttctaaaatttcatcttgaatccaaattttaatttttgaatgactttccctgctgttgtcttcaaaatcagaacattttctctgcctcagaaaagcgtttttccaactggaaatttatttttcaggtcttaaaacctgctaaatgtttttaggaagtacctactgaaactttttgtaagacatttttggaacgagcttgaacatttatataaatttattaccctctttgatttttgaaacatgcatattatatttaggctgagaagcccttcaaatggccagataagccacagttttagctagagaaccatttagaattgacataactaatctaaacttgaacacttttaggaccaatgttagtgttctaaataccaacatatttctgatgtttaaacagatctcccaaattcttaggaccttgatgtcattaaaatttagaatgacaagcttaagaggctttagtttcatttgtttttcaagtaatgaaaaataatttcttacatgggcagatagttaatttgttgaacaattacaggtagcatttcatgtaatctgatgttctaaatggttctcttattgaaggaggttaaagaattaggtttcttacagtttttggctggccatgacatgtataaaatgtatattaaggaggaattataaagtactttaatttgaatgctagtggcaattgatcattaagaaagtactttaaagcaaaaggttaatgggtcatctgggaaaaatactgaagtatcaaaggtatttgcatgtgaatgtgggttatgttcttctatcccaccttgtagcatattctatgaaagttgagttaaatgatagctaaaatatctgtttcaacagcatgtaaaaagttattttaactgttacaagtcattatacaattttgaatgttctgtagtttctttttaacagtttaggtacaaaggtctgttttcattctggtgctttttattaattttgatagtatgatgtcacttcctattgaaatgtaagctagcgtgtaccttagaatgtgagctccatgagagcaggtaccttgtttgtcttcactgctgtatctattcccaacgcctcatgacagtgcctggcacatagtaggcactcaataaatacttgttgaatgaatgaaaaaaaaaaaaaa

SEQ ID NO: 28 - protein, 692 aa, Mus musculus; identical to SEQ ID NO: 26

SEQ ID NO: 29 - DNA, 2109 bp, Gallus gallus
  source 1..2109: mol_type = unassigned DNA
  CDS 1..2109
atgccctcggctaccaacggcaccatggcgagcagcagcgggaaggcgggcccgggcggcaacgagcaggccccggcggcggcagcggcggccccgcaggcgtcgggcggcagcatcacctcggttcagaccgaggccatgaagcagatcttgggagtgatcgacaaaaagctccgcaacctcgagaagaaaaagagcaaacttgacgattaccaggaacgaatgaacaagggggaacgtctaaatcaagatcaactggatgcagtgtcaaaataccaggaagtgacaaataacctggaattcgctaaagaactgcagaggagctttatggcactgagccaagatatccagaaaacaataaaaaagacggctcgcagggagcagctgatgagagaagaggctgagcagaagcgtttaaagactgtgctagagctgcagttcattttggacaagttgggtgacgatgaagtgcgcagtgacttgaaacaaggatcaaatggagtaccggtactgacagaggaggaactgacaatgctggatgaattttacaagctagtttaccctgaaagggacatgaacatgaggttgaatgagcagtatgagcaagcatctgttcacctgtgggacttactggaagggaaggaaaaacccgtttgtggaacaacctataaagccctgaaggaggttgttgaacgtattcttcaaactagttactttgatagcacccataaccatcagaacgggttatgtgaggaagaagaggcagcacccacacctgcagtagaagacactgtagcagaagctgagcctgatccagcagaagaatttactgaacctactgaagttgaatcgactgagtatgtaaacagacaattcatggcagagactcagttcagcagtagtgagaaggaacaggtagatgagtggacagttgaaacggttgaggttgtaaattcactgcagcaacaaacacaagctacatctcctccagttcctgaacctcatacactcactactgtggctcaagcagatcctcttgttagaagacagagagtacaggaccttatggcccagatgcagggtccatataacttcatgcaggactctatgctggagtttgagaaccagacacttgatcctgccattgtatctgcacagcccatgaatccagcacagaatttggacatgccgcaaatggtctgccctccagttcatactgagtcaagacttgcccagcctaatcaagttcctgtgcaaccagaagctacgcaggttcccttggtttcatctacaagtgagggatatacagcctcccagcccatgtatcagccttctcataccacagagcaacggccacagaaggaatccattgaccagattcaggcttcaatgtcactgaatgcagaccagaccccgtcatcatcatcacttcccactgcatcccagccgcaagttttccaagctggatctagcaaacctttgcatagcagcggaatcaatgttaatgcagctccattccaatccatgcaaacagtattcaacatgaatgcacctgttcctcctgttaatgagccagaagcccttaagcaacaaaatcagtaccaggccagttacaaccagagtttctccaatcagccacaccaagtagaacaatcagatcttcagcaagaacagctccagacagtggttggtacttaccatggttctccggaccagacccatcaagtggcaggaaaccaccagcaacctccccagcagaatactggatttccacgcaacagtcagccttattacaacagtcggggagtgtctcgtggtggatcacgtgggactcgtggattgatgaatggttacaggggacctgcaaatggatttagaggaggatatgatggctaccgtccttcattttccaacactccgaacagtggttacacgcagccccaatttaatgctcctcgagattattcaaactaccagcgggatggatatcagcagaacttcaaacgtggttctggacaaagtgggcctcggggagctcctcgaggtcgtggagggcccccaagaccaaacagagggatgcctcaaatgaacgctcagcaagtgaattaa

SEQ ID NO: 30 - protein, 702 aa, Gallus gallus
MPSATNGTMASSSGKAGPGGNEQAPAAAAAAPQASGGSITSVQTEAMKQILGVIDKKLRNLEKKKSKLDDYQERMNKGERLNQDQLDAVSKYQEVTNNLEFAKELQRSFMALSQDIQKTIKKTARREQLMREEAEQKRLKTVLELQFILDKLGDDEVRSDLKQGSNGVPVLTEEELTMLDEFYKLVYPERDMNMRLNEQYEQASVHLWDLLEGKEKPVCGTTYKALKEVVERILQTSYFDSTHNHQNGLCEEEEAAPTPAVEDTVAEAEPDPAEEFTEPTEVESTEYVNRQFMAETQFSSSEKEQVDEWTVETVEVVNSLQQQTQATSPPVPEPHTLTTVAQADPLVRRQRVQDLMAQMQGPYNFMQDSMLEFENQTLDPAIVSAQPMNPAQNLDMPQMVCPPVHTESRLAQPNQVPVQPEATQVPLVSSTSEGYTASQPMYQPSHTTEQRPQKESIDQIQASMSLNADQTPSSSSLPTASQPQVFQAGSSKPLHSSGINVNAAPFQSMQTVFNMNAPVPPVNEPEALKQQNQYQASYNQSFSNQPHQVEQSDLQQEQLQTVVGTYHGSPDQTHQVAGNHQQPPQQNTGFPRNSQPYYNSRGVSRGGSRGTRGLMNGYRGPANGFRGGYDGYRPSFSNTPNSGYTQPQFNAPRDYSNYQRDGYQQNFKRGSGQSGPRGAPRGRGGPPRPNRGMPQMNAQQVN

SEQ ID NO: 31 - DNA, 20 bp, synthetic construct
  misc_feature 1..20: note = T3 primer
  source 1..20: mol_type = other DNA
aattaaccctcactaaaggg

SEQ ID NO: 32 - DNA, 19 bp, synthetic construct
  misc_feature 1..19: note = T7 primer
  source 1..19: mol_type = other DNA
taatacgactcactatagg

SEQ ID NO: 33 - DNA, 18 bp, synthetic construct
  misc_feature 1..18: note = primer
  source 1..18: mol_type = other DNA
aaggtttgaatggagtgc

SEQ ID NO: 34 - DNA, 18 bp, synthetic construct
  misc_feature 1..18: note = primer
  source 1..18: mol_type = other DNA
tgctccttttcaccactg

SEQ ID NO: 35 - DNA, 18 bp, synthetic construct
  misc_feature 1..18: note = GAPDH primer
  source 1..18: mol_type = other DNA
gggctgcttttaactctg

SEQ ID NO: 36 - DNA, 18 bp, synthetic construct
  misc_feature 1..18: note = GAPDH primer
  source 1..18: mol_type = other DNA
ccaggaaatgagcttgac

SEQ ID NO: 37 - protein, 14 aa, synthetic construct
  REGION 1..14: note = peptides
RNLEKKKGKLDDYQ

SEQ ID NO: 38 - DNA, 22 bp, synthetic construct
  misc_feature 1..22: note = primer
  source 1..22: mol_type = other DNA
aggtsharctgcagsagtcwgg

SEQ ID NO: 39 - DNA, 23 bp, synthetic construct
  misc_feature 1..23: note = primer
  source 1..23: mol_type = other DNA
ctcgagttaattcacttgctgag

SEQ ID NO: 40 - protein, 5 aa, Mus musculus
DYNMD

SEQ ID NO: 41 - protein, 17 aa, Mus musculus
DINPNYDSTSYNQKFKG

SEQ ID NO: 42 - protein, 11 aa, Mus musculus
SRSYDYEGFAY

SEQ ID NO: 43 - protein, 148 aa, Mus musculus
MEWSGVFIFLLSGTAGVLSEVQLHQFGAELVKPGASVKISCKASGYTFTDYNMDWVKQSHGKSLEWIGDINPNYDSTSYNQKFKGKATLTVDKSSSTAYMELRSLTSEDTAVYYCARSRSYDYEGFAYWGQGTLVTVSAAKTTPPSVY

SEQ ID NO: 44 - protein, 11 aa, Mus musculus
RASGNIHNYLA

SEQ ID NO: 45 - protein, 7 aa, Mus musculus
NAKTLAD

SEQ ID NO: 46 - protein, 8 aa, Mus musculus
QHFWSTLT

SEQ ID NO: 47 - protein, 139 aa, Mus musculus
MSVLTQVLGLLLLWLTGARCDIQMTQSPASLSASVGETVTITCRASGNIHNYLAWYQQKQGKSPQLLVYNAKTLADGVPSRFSGSGSGTQYSLKINSLQPEDFGSYYCQHFWSTLTFGAGTKLELKRADAAPTVSNPYD

SEQ ID NO: 48 - DNA, 444 bp, Mus musculus
  source 1..444: mol_type = unassigned DNA
atggaatggagcggggtctttatctttctcctgtcaggaactgcaggtgtcctctctgaggtccagctgcatcagtttggagctgagctggtgaagcctggggcttcagtgaagatatcctgcaaggcttctggctacacattcactgactacaacatggactgggtgaagcagagccatggaaagagccttgagtggattggagatattaatcctaactatgatagtactagctacaaccagaagttcaagggaaaggccacattgactgtagacaagtcctccagcacagcctacatggagctccgcagcctgacatctgaggacactgcagtctattactgtgcaagatcgaggagctatgattacgaaggatttgcttactggggccaagggactctggtcactgtctctgcagccaaaacaacacccccatcagtctat

SEQ ID NO: 49 - DNA, 444 bp, Mus musculus; identical to SEQ ID NO: 48
  source 1..444: mol_type = unassigned DNA

SEQ ID NO: 50 - protein, 11 aa, Mus musculus
LWSVNQKNYLS

SEQ ID NO: 51 - protein, 7 aa, Mus musculus
GASIRES

SEQ ID NO: 52 - protein, 9 aa, Mus musculus
QHNHGSFLP

SEQ ID NO: 53 - protein, 132 aa, Mus musculus
AVLRCSRGLLVIWISDIQLTQSPSSLAVTAGEKVTMSCKSSQSLLWSVNQKNYLSWYQQKQRQPPKLLIYGASIRESWVPDRFTGSGSGTDFTLTISNVHAEDLAVYYCQHNHGSFLPSRSEQVPSWRSNNR

SEQ ID NO: 54 - DNA, 398 bp, Mus musculus
  source 1..398: mol_type = unassigned DNA
gcggtcctgcggtgctctagaggactactagtcatatggatttccgatatccagctgacccagtctccatcctccctggctgtgacagcaggagagaaggtcactatgagctgcaagtccagtcagagtcttttgtggagtgtaaaccagaagaactacttgtcctggtaccagcagaaacaaaggcagcctcctaaactgcttatctatggggcatccattagagaatcttgggtccctgatcggttcacaggaagtggatctgggacagacttcactctcaccattagcaatgtgcatgctgaagacctagcagtttattactgtcaacacaatcatggcagctttctcccctcacgttcggagcaggtaccaagctggagatcaaacaatcggat

SEQ ID NO: 55 - protein, 11 aa, Mus musculus
RASGNIHNYLA

SEQ ID NO: 56 - protein, 7 aa, Mus musculus
NAKTLAD

SEQ ID NO: 57 - protein, 9 aa, Mus musculus
QHFWSTLTF

SEQ ID NO: 58 - protein, 117 aa, Mus musculus
RTTSHMDSDIQLTQSPASLSASVGETVTITCRASGNIHNYLAWYQQKQGKSPQLLVYNAKTLADGVPSRFSGSGSGTQYSLKINSLQPEDFGSYYCQHFWSTLTFGGGTKLEIKQSD

SEQ ID NO: 59 - DNA, 353 bp, Mus musculus
  source 1..353: mol_type = unassigned DNA
gaggactactagtcatatggattccgatatccagctgacccagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtacgctcacgttcggaggtggtaccaagctggagatcaaacaatcggatc

SEQ ID NO: 60 - protein, 11 aa, Mus musculus
RASQSISNYLH

SEQ ID NO: 61 - protein, 7 aa, Mus musculus
YASQSIS

SEQ ID NO: 62 - protein, 9 aa, Mus musculus
QQSNSWPYT

SEQ ID NO: 63 - protein, 94 aa, Mus musculus
SGDRVSLSCRASQSISNYLHWYQQKSHESPRLLIKYASQSISGIPSRFSGSGSGTDFTLSINSVETEDFGMYFCQQSNSWPYTFGAGTKLEIKQ

SEQ ID NO: 64 - DNA, 283 bp, Mus musculus
  source 1..283: mol_type = unassigned DNA
tcaggagatagagtcagtctttcctgcagggccagtcaaagtattagcaactacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggccgtacacgttcggtgcaggtaccaagctggagatcaaacaga

SEQ ID NO: 65 - protein, 11 aa, Mus musculus
LSIVNRYHYMS

SEQ ID NO: 66 - protein, 6 aa, Mus musculus
EASITK

SEQ ID NO: 67 - protein, 9 aa, Mus musculus
QHNRGSFLP

SEQ ID NO: 68 - protein, 105 aa, Mus musculus
GLFCSVERCHYQLQSSQNLLSIVNRYHYMSGNPPKLLVYPALLIYEASITKSCVPDRFTRSGSGTNFTLTINFVHADDLIFYYCQHNRGSFLPSSSVQVPRRRSN

SEQ ID NO: 69 - DNA, 317 bp, Mus musculus
  source 1..317: mol_type = unassigned DNA
ggactcttctgctctgtggagagatgtcactatcaactgcaatccagtcagaatcttttgagtattgtaaaccggtatcactacatgtccggaaaccctcctaaactcctggtctatcctgcactgcttatctatgaggcatccattacaaaatcctgtgtccctgatcggttcacacgaagtggatctgggacaaacttcactctcaccattaattttgtgcatgctgatgacctaattttttattactgtcaacacaatcgtggcagctttctcccctcaagttcggtgcaggtaccaagaaggagatcaaacaa

SEQ ID NO: 70 - protein, 5 aa, Mus musculus
GYTMN

SEQ ID NO: 71 - protein, 16 aa, Mus musculus
NPYNGGTSYNQKFKGK

SEQ ID NO: 72 - protein, 9 aa, Mus musculus
WGVWSAMDY

SEQ ID NO: 73 - protein, 100 aa, Mus musculus
DILQASGYSFTGYTMNWVKQSHGKNLEWIGLINPYNGGTSYNQKFKGKATLTVDKSSSTAYMELLSLTSEDSAVYYCARWGVWSAMDYWGQGTTVTVSSK

SEQ ID NO: 74 - protein, 11 aa, Mus musculus
KASQNVRTAVA

SEQ ID NO: 75 - protein, 7 aa, Mus musculus
LASNRDT

SEQ ID NO: 76 - protein, 9 aa, Mus musculus
LQHCNYPNE

SEQ ID NO: 77 - protein, 90 aa, Mus musculus
DRVSITCKASQNVRTAVAWYQQKPRQSPKALIYLASNRDTGLPDRFPGRGSGTDFTLNITNVQSEDLEDYFCLQHCNYPNEFRGCTKVPI

SEQ ID NO: 78 - DNA, 301 bp, Mus musculus
  source 1..301: mol_type = unassigned DNA
gatatcctgcaggcttctggttactcattcactggctacaccatgaactgggtgaagcagagccatggaaagaaccttgagtggattggacttattaatccttacaatggtggtactagctacaaccagaagttcaagggcaaggccacattaactgtagacaagtcatccagcacagcctacatggagctcctcagtctgacatctgaggactctgcagtctattactgtgcaagatggggggtatggtcggctatggactactggggccaagggaccacggtcaccgtctcctcaaaaa

SEQ ID NO: 79 - DNA, 290 bp, Mus musculus
  source 1..290: mol_type = unassigned DNA
gacagggtcagcatcacctgcaaggccagtcaaaatgttcgtactgctgtagcctggtatcaacagaaaccacggcagtctcctaaagcactgatttacttggcatccaaccgggacactggactccctgatcgcttcccaggcaggggatctgggacagatttcactctcaacattaccaatgtgcaatctgaagacctggaagattatttctgtctgcaacattgtaattatcctaacgagttcagaggttgtaccaaggtgccaatctaaagaacaaacaccccctg

SEQ ID NO: 80 - protein, 5 aa, Mus musculus
SYWMQ

SEQ ID NO: 81 - protein, 17 aa, Mus musculus
AIYPGDGDTRYTQKFKG

SEQ ID NO: 82 - protein, 11 aa, Mus musculus
ARGEYGNYFAY

SEQ ID NO: 83 - protein, 116 aa, Mus musculus
LQESGAELARPGASVKLSCKASGYTFTSYWMQWVKQRPGQGLEWIGAIYPGDGDTRYTQKFKGKATLTADKSSSTAYMQLSSLASEDSAVYYCARGEYGNYFAYWGQGTTVTVSSN

SEQ ID NO: 84 - protein, 11 aa, Mus musculus
KASQDINSYLS

SEQ ID NO: 85 - protein, 7 aa, Mus musculus
RANRLVD

SEQ ID NO: 86 - protein, 9 aa, Mus musculus
LQYDEFPLT

SEQ ID NO: 87 - protein, 100 aa, Mus musculus
TSDASLGERVTITCKASQDINSYLSWFQQKPGKSPKTLIYRANRLVDGVPSRFSGSGSGQDYSLTISSLEYEDMGIYYCLQYDEFPLTFGGGTKLEIKQK

SEQ ID NO: 88 - DNA, 352 bp, Mus musculus
  source 1..352: mol_type = unassigned DNA
aactgcaggagtctggggctgagctggcaagacctggggcttcagtgaagttgtcctgcaaggcttctggctacacctttactagctactggatgcagtgggtaaaacagaggcctggacagggtctggaatggattggggctatttatcctggagatggtgatactaggtacactcagaagttcaagggcaaggccacattgactgcagataaatcctccagcacagcctacatgcaactcagcagcttggcatctgaggactctgcggtctattactgtgcaagaggggagtatggtaactattttgcttactggggccaagggaccacggtcaccgtctcctcaaatcg

SEQ ID NO: 89 - DNA, 302 bp, Mus musculus
  source 1..302: mol_type = unassigned DNA
ggacatcggatgcatctctaggagagagagtcactatcacttgcaaggcgagtcaggacattaatagctatttaagctggttccagcagaaaccagggaaatctcctaagaccctgatctatcgtgcaaacagattggtagatggggtcccatcaaggttcagtggcagtggatctgggcaagattattctctcaccatcagcagcctggagtatgaagatatgggaatttattattgtctacagtatgatgagtttccgctcacgttcggaggaggtaccaagctggagatcaaacaaaaa

SEQ ID NO: 90 - protein, 5 aa, Mus musculus
DTYMH

SEQ ID NO: 91 - protein, 17 aa, Mus musculus
RIDPANGNTKYDPKFQG

SEQ ID NO: 92 - protein, 14 aa, Mus musculus
ARPIHYYYGSSLAY

SEQ ID NO: 93 - protein, 108 aa, Mus musculus
AWLSQLSCTASGFNIKDTYMHWVKQRPEQGLEWIGRIDPANGNTKYDPKFQGKATITADTSSNTAYLQLSSLTSEDTAVYYCARPIHYYYGSSLAYWGQGTTVTVSSK

SEQ ID NO: 94 - protein, 11 aa, Mus musculus
SVDSYGNSFMH

SEQ ID NO: 95 - protein, 7 aa, Mus musculus
RASNLES

SEQ ID NO: 96 - protein, 9 aa, Mus musculus
QQSNEDPGR

SEQ ID NO: 97 - protein, 104 aa, Mus musculus
EFHAVSLGQRATISCRASESVDSYGNSFMHWYQQKPGQPPKLLIYRASNLESGIPARFSGSGSRTDFTLTINPVEADDVATYYCQQSNEDPGRSEVVPSWRSNK

SEQ ID NO: 98 - DNA, 326 bp, Mus musculus
  source 1..326: mol_type = unassigned DNA
gcatggctcagtcagttgtcctgcacagcttctggcttcaacattaaagacacctatatgcactgggtgaagcagaggcctgaacagggcctggagtggattggaaggattgatcctgcgaatggtaatactaaatatgacccgaagttccagggcaaggccactataacagcagacacatcctccaacacagcctacctgcagctcagcagcctgacatctgaggacactgccgtctattactgtgctagaccgattcattattactacggtagtagccttgcttactggggccaagggaccacggtcaccgtctcctcaaaaaa

SEQ ID NO: 99 - DNA, 313 bp, Mus musculus
  source 1..313: mol_type = unassigned DNA
gagtttcatgctgtgtctctagggcagagggccaccatatcctgcagagccagtgaaagtgttgatagttatggcaatagttttatgcactggtaccagcagaaaccaggacagccacccaaactcctcatctatcgtgcatccaacctagaatctgggatccctgccaggttcagtggcagtgggtctaggacagacttcaccctcaccattaatcctgtggaggctgatgatgttgcaacctattactgtcagcaaagtaatgaggatcctggacgttcggaggtggtaccaagctggagatcaaacaaaa

SEQ ID NO: 100 - protein, 5 aa, Mus musculus
DYYMS

SEQ ID NO: 101 - protein, 17 aa, Mus musculus
RNKANGYTTEYSASVKG

SEQ ID NO: 102 - protein, 9 aa, Mus musculus
ARANWAFDY

SEQ ID NO: 103 - protein, 109 aa, Mus musculus
PRASLGVSETLLCTSGFTFTDYYMSWVRQPPGKALEWLGFIRNKANGYTTEYSASVKGRFTISRDNSQSILYLQMNTLRAEDSATYYCARANWAFDYWGQGTTVTVSSK

SEQ ID NO: 104 - protein, 11 aa, Mus musculus
RASQSISNYLH

SEQ ID NO: 105 - protein, 7 aa, Mus musculus
YASQSIS

SEQ ID NO: 106 - protein, 9 aa, Mus musculus
QQSNSWPYT

SEQ ID NO: 107 - protein, 94 aa, Mus musculus
SGDRVSLSCRASQSISNYLHWYQQKSHESPRLLIKYASQSISGIPSRFSGSGSGTDFTLSINSVETEDFGMYFCQQSNSWPYTFGGGTKLEIKQ

SEQ ID NO: 108 - DNA, 329 bp, Mus musculus
  source 1..329: mol_type = unassigned DNA
ggccgcgtgctagcctgggggtctctgagactctcctgtgcacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatgaacaccctgagagctgaggacagtgccacttattactgtgcaagggctaactgggcctttgactactggggccaagggaccacggtcaccgtctcctcaaaa

SEQ ID NO: 109 - DNA, 284 bp, Mus musculus
  source 1..284: mol_type = unassigned DNA
tcaggagatagagtcagtctttcctgcagggccagtcaaagtattagcaactacctacactggtatcaacaaaaatcacatgagtctccaaggcttctcatcaagtatgcttcccagtccatctctgggatcccctccaggttcagtggcagtggatcagggacagatttcactctcagtatcaacagtgtggagactgaagattttggaatgtatttctgtcaacagagtaacagctggccgtacacgttcggaggaggtaccaagctggagatcaaacagaa

SEQ ID NO: 110 - protein, 5 aa, Mus musculus
DYYMS

SEQ ID NO: 111 - protein, 17 aa, Mus musculus
RNKANGYTTEYSASVKG

SEQ ID NO: 112 - protein, 12 aa, Mus musculus
ARAPLLYYAMDY

SEQ ID NO: 113 - protein, 111 aa, Mus musculus
PACLPGGSLRLSCATSGFTFTDYYMSWVRQPPGKALEWLGFIRNKANGYTTEYSASVKGRFTISRDNSQSILYLQMNTLRAEDSATYYCARAPLLYYAMDYWGQGTTVTVS

SEQ ID NO: 114 - protein, 11 aa, Mus musculus
NVSTSGYSYMH

SEQ ID NO: 115 - protein, 7 aa, Mus musculus
LVSNLES

SEQ ID NO: 116 - protein, 8 aa, Mus musculus
QHIRELTR

SEQ ID NO: 117 - protein, 102 aa, Mus musculus
RLPFYSLEQRATISYRASKNVSTSGYSYMHWNQQKPGQPPKLLIYLVSNLESGVPARFSGSGSGTDFTLNIHPVEEEDAATYYCQHIRELTRSELVPSWKSN

SEQ ID NO: 118 - DNA, 340 bp, Mus musculus
  source 1..340: mol_type = unassigned DNA
ccggcctgcttgcctggtggttctctgagactctcctgtgcaacttctgggttcaccttcactgattactacatgagctgggtccgccagcctccaggaaaggcacttgagtggttgggttttattagaaacaaagctaatggttacacaacagagtacagtgcatctgtgaagggtcggttcaccatctccagagataattcccaaagcatcctctatcttcaaatgaacaccctgagagctgaggacagtgccacttattactgtgcaagagcccctctactttactatgctatggactactggggccaagggaccacggtcaccgtctcctaaatta

SEQ ID NO: 119 - DNA, 306 bp, Mus musculus
  source 1..306: mol_type = unassigned DNA
cgccttcctttctattctctggagcagagggccaccatctcatacagggccagcaaaaatgtcagtacatctggctatagttatatgcactggaaccaacagaaaccaggacagccacccaaactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggagctggtaccaagctggaaatcaaac

SEQ ID NO: 120 - protein, 5 aa, Mus musculus
SYWMH

SEQ ID NO: 121 - protein, 17 aa, Mus musculus
MIDPSNSETRLNQKFKD

SEQ ID NO: 122 - protein, 12 aa, Mus musculus
ARGLRHYWYFDV

SEQ ID NO: 123 - protein, 101 aa, Mus musculus
VSCKASGYTFTSYWMHWVKQRPGQGLEWIGMIDPSNSETRLNQKFKDKATLNVDKSSNTAYMQLSSLTSEDSAVYYCARGLRHYWYFDVWGQGTTVTVSSK

SEQ ID NO: 124 - protein, 11 aa, Mus musculus
SVSTSGYSYMH

SEQ ID NO: 125 - protein, 7 aa, Mus musculus
LVSNLES

SEQ ID NO: 126 - protein, 9 aa, Mus musculus
QHIRELTRS

SEQ ID NO: 127 - protein, 99 aa, Mus musculus
TILWREGPFSYRASKSVSTSGYSYMHWNQQKPGQPPRLLIYLVSNLESGVPARFSGSGSGTDFTLNIHPVEEEDAATYYCQHIRELTRSEEVPSWRSNK

SEQ ID NO: 128 - DNA, 304 bp, Mus musculus
  source 1..304: mol_type = unassigned DNA
gtgtcctgcaaggcttcaggctataccttcaccagctactggatgcactgggtgaaacagaggcctggacaaggccttgagtggattggcatgattgatccttccaatagtgaaactaggttaaatcagaagttcaaggacaaggccacattgaatgtagacaaatcctccaacacagcctacatgcagctcagcagcctgacatctgaggactctgcagtctattactgtgcaagagggttacgccactactggtacttcgatgtctggggccaagggaccacggtcaccgtctcctcaaaaa

SEQ ID NO: 129 - DNA, 298 bp, Mus musculus
  source 1..298: mol_type = unassigned DNA
actattctctggagagagggccccttctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggaggtaccaagctggagatcaaacaaaa

SEQ ID NO: 130 - DNA, 22 bp, synthetic construct
  misc_feature 1..22: note = primer
  source 1..22: mol_type = other DNA
aggtsharctgcagsagtcwgg

SEQ ID NO: 131 - DNA, 34 bp, synthetic construct
  misc_feature 1..34: note = primer
  source 1..34: mol_type = other DNA
tgaggagacggtgaccgtggtcccttggccccag

SEQ ID NO: 132 - DNA, 27 bp, synthetic construct
  misc_feature 1..27: note = primer
  source 1..27: mol_type = other DNA
tccgatatccagctgacccagtctcca

SEQ ID NO: 133 - DNA, 31 bp, synthetic construct
  misc_feature 1..31: note = primer
  source 1..31: mol_type = other DNA
gtttgatctccagcttggtacchscdccgaa

SEQ ID NO: 134 - DNA, 15 bp, synthetic construct
  misc_feature 1..15: note = primer
  source 1..15: mol_type = other DNA
agtcacgacgttgta

SEQ ID NO: 135 - DNA, 17 bp, synthetic construct
  misc_feature 1..17: note = primer
  source 1..17: mol_type = other DNA
caggaaacagctatgac

SEQ ID NO: 136 - protein, 58 aa, Homo sapiens
VFQSNYFDSTHNHQNGLCEEEEAASAPAVEDQVPEAEPEPAEEYTEQSEVESTEYVNR

SEQ ID NO: 137 - protein, 15 aa, Homo sapiens
YTEQSEVESTEYVNR

SEQ ID NO: 138 - protein, 11 aa, Homo sapiens
SEVESTEYVNR

(19) United States
(12) Patent Application Publication
(10) Pub. No.: US 2026/0176372 A1
(43) Pub. Date: Jun. 25, 2026

(54) PHARMACEUTICAL COMPOSITION FOR TREATMENT AND PREVENTION OF CANCERS

(71) Applicant: TORAY INDUSTRIES, INC., Tokyo (JP)

(72) Inventors: Fumiyoshi OKANO, Kamakura-shi (JP); Takayoshi IDO, Kamakura-shi (JP); Takanori SAITO, Kamakura-shi (JP); Shinichi KOBAYASHI, Kamakura-shi (JP)

(73) Assignee: TORAY INDUSTRIES, INC., Tokyo (JP)

(21) Appl. No.: 19/544,015

(22) Filed: Feb. 19, 2026

Related U.S. Application Data
(62) Division of application No. 17/177,396, filed on Feb. 17, 2021.
Application No. 17/177,396 is a division of application No. 15/971,647, filed on May 4, 2018, now abandoned.
Application No. 15/971,647 is a division of application No. 15/092,469, filed on Apr. 6, 2016, now Pat. No. 9,982,059.
Application No. 15/092,469 is a division of application No. 13/057,709, now Pat. No. 9,416,192.

(30) Foreign Application Priority Data
Aug. 5, 2008 (JP) 2008-201928
Mar. 31, 2009 (JP) 2009-087285

(51) Int. Cl. C07K 16/30; A61K 39/00; A61K 39/395

(52) U.S. Cl. CPC C07K 16/3015; A61K 39/39558; C07K 16/30; C07K 16/3053; A61K 2039/505; C07K 2317/24; C07K 2317/34; C07K 2317/565; C07K 2317/732; C07K 2317/734

(57) ABSTRACT

The present invention relates to a pharmaceutical composition for treatment and/or prevention of cancer, which comprises, as an active ingredient, an antibody or fragment thereof having an immunological reactivity with a CAPRIN-1 protein or a fragment thereof comprising 7 or more consecutive amino acids.

### CROSS-REFERENCE TO RELATED APPLICATIONS

This application is a Divisional of copending U.S. application Ser. No. 17/177,396 filed Feb. 17, 2021, which is a Divisional of U.S. application Ser. No. 15/971,647 filed May 4, 2018 (abandoned), which is a Divisional of U.S. application Ser. No. 15/092,469, filed on Apr. 6, 2016, now U.S. Pat. No. 9,982,059 issued May 29, 2018, which is a Divisional of U.S. application Ser. No. 13/057,709, filed on Feb. 4, 2011, now U.S. Pat. No. 9,416,192, issued Aug. 16, 2016, which the National Phase of PCT International Application No. PCT/JP2009/063882 filed on Aug. 5, 2009, which claims priority on Japanese Patent Application Nos. 2009-087285 filed on Mar. 31, 2009, and 2008-201928 filed on Aug. 5, 2008, all of which are hereby expressly incorporated by reference into the present application.

### REFERENCE TO SEQUENCE LISTING SUBMITTED VIA EFS-WEB

This application includes an electronically submitted sequence listing in .xml format. The electronic CRF of the Sequence Listing, file “PH-4051-PCT-US04”, was created on Feb. 5, 2026, and is 206,823 bytes in size. The sequence listing contained in this .xml file is part of the specification and is hereby incorporated by reference herein in its entirety.

### TECHNICAL FIELD

The present invention relates to a novel medical use of antibodies to CAPRIN-1 or fragments thereof as, for example, therapeutic and/or preventive agents for cancer.

### BACKGROUND OF INVENTION

Cancer is the leading cause of death. Treatment currently performed for cancer is mainly surgical therapy, which can be combined with radiation therapy or chemotherapy. In spite of development of new surgical methods and discovery of new anti-cancer agents in recent years, treatment results of cancers are not greatly improved at present except for some cancers. Through a recent progress of molecular biology and cancer immunology, antibodies that are specifically reactive with cancers, cancer antigens recognized by cytotoxic T cells, as well as the genes encoding the cancer antigens, have been identified, and expectations for specific immunotherapies targeting cancer antigens have been raised (Tsuyoshi AKIYOSHI, “Gan To Kagaku-Ryoho (Cancer and Chemotherapy),” 1997, vol. 24, pp. 551-519 (Jp) (Cancer and Chemotherapy Publishers, Inc., Japan)).

In cancer treatment methods, in order to reduce side effects, it is desirable for peptides, polypeptides, or proteins recognized as cancer antigens to be absent in almost all normal cells but specifically present in cancer cells. In 1991, Boon et al of the Ludwig Institute in Belgium isolated the human melanoma antigen MAGE 1 recognized by CD8-positive T cells by the cDNA-expression cloning method using an autologous cancer cell line and cancer-reactive T cells (Bruggen P. et al., Science, 254:1643-1647 (1991)). Thereafter, the SEREX (serological identification of antigens by recombinant expression cloning) method was reported, wherein tumor antigens recognized by antibodies produced through response to an autologous cancer in the body of a patient with cancer can be identified using the gene-expression cloning technique (Proc. Natl. Acad. Sci. USA, 92:11810-11813 (1995); and U.S. Pat. No. 5,698,396). By the SEREX method, some cancer antigens, which are not substantially expressed in normal cells but are specifically expressed in cancer cells, were isolated (Int. J. Cancer, 72: 965-971 (1997); Cancer Res., 58: 1034-1041 (1998); Int. J. Cancer, 29: 652-658 (1998); Int. J. Oncol., 14: 703-708 (1999); Cancer Res., 56: 4766-4772 (1996); and Hum. Mol. Genet 6: 33-39, 1997). Further, clinical trials of cell therapies using immunocytes that specifically react with cancer antigens, which are some of the isolated cancer antigens, and cancer-specific immunotherapies using vaccines comprising cancer antigens or the like have been conducted.

Meanwhile, in recent years, a variety of antibody medicines for cancer treatment that target antigen proteins on cancer cells have come into existence. Such medicines used as cancer-specific therapeutic agents exhibit drug efficacy to a certain extent, and thus they have been gaining attention. However, most of target antigen proteins are also expressed on normal cells. As a result of antibody administration, not only cancer cells, but also normal cells, on which a target antigen has been expressed can be damaged, thereby causing a side (or adverse) effect, which becomes problematic. Hence, it is expected that, if it becomes possible to identify cancer antigens that are specifically expressed on the surface of a cancer cell and to use antibodies targeting such antigens as medicaments, then treatment with antibody medicines that cause fewer side effects could be realized.

Cytoplasmic- and proliferation-associated protein 1 (CAPRIN-1) is an intracellular protein that is expressed when normal cells in resting phase are activated or undergo cell division. CAPRIN-1 is also known to be involved in the regulation of the transport and translation of mRNAs through formation of cytoplasmic stress granules with RNA in a cell. CAPRIN-1 has different names, such as GPI-anchored membrane protein 1 and membrane component surface marker 1 protein (M11S1), as if this protein is known to be a membrane protein. These different names are derived from the report (J. Biol. Chem., 270: 20717-20723, 1995) that the gene sequence of CAPRIN-1 originally has a GPI-binding region and CAPRIN-1 is a membrane protein expressed in colon cancer cells. It was later reported that the CAPRIN-1 gene sequence described in said report was not correct; i.e., a frame shift took place by deletion of a single nucleotide from the CAPRIN-1 gene sequence currently registered with GenBank or the like, so that 80 amino acids were deleted from the C-terminus and the resulting artifact (74 amino acids) was the GPI binding portion in the report; and another error was also present on the 5′ side of the gene sequence, thereby resulting in deletion of 53 amino acids from the N-terminus (J. Immunol., 172: 2389-2400, 2004). Further, it has been reported that the protein encoded by the CAPRIN-1 gene sequence currently registered with GenBank or the like was not a cell membrane protein (J. Immunol., 172: 2389-2400, 2004).

In addition, based on the report of J. Biol. Chem., 270: 20717-20723, 1995 that CAPRIN-1 is a cell membrane protein, US2008/0075722 and WO2005/100998 disclose that CAPRIN-1 under the name of M11S1 can be used for cancer therapy as a target of antibody medicines for cancer therapy and as one of cell membrane proteins; however, the Examples contain no description of the cancer therapy using an antibody against the protein. However, as reported in J. Immunol., 172: 2389-2400, 2004, it was a common belief, from the time of filing US2008/0075722 up to now, that CAPRIN-1 is not expressed on the surface of a cell, and thus, it is obvious that the contents of US2008/0075722 and WO2005/100998 based only on misinformation that CAPRIN-1 is a cell membrane protein should not be understood as common technical knowledge of persons skilled in the art.

### SUMMARY OF THE INVENTION

### Problem to be Solved by the Invention

An object of the present invention is to identify cancer antigen proteins specifically expressed on the surface of cancer cells and to provide a use of antibodies targeting such proteins as therapeutic and/or preventive (or prophylactic) agents for cancer.

### Means for Solving Problem

As a result of intensive studies, the present inventors have now obtained cDNA encoding a protein that binds to an antibody present in the serum from a tumor-bearing organism by the SEREX method using testis tissue-derived cDNA libraries and sera from dogs with breast cancer. With the use of the obtained canine genes and genes homologous thereto from human, bovine, horse, mouse, and chicken, CAPRIN-1 proteins having amino acid sequences shown in the even numbers of SEQ ID NOS: 2 to 30 (i.e., even-numbered SEQ ID NOS: 2 to 30) and antibodies against the CAPRIN-1 proteins have now been prepared. In addition, the present inventors have now found that CAPRIN-1 is specifically expressed in the cells of breast cancer, brain tumor, leukemia, lymphoma, lung cancer, esophageal cancer, colon cancer, gastric cancer, and kidney cancer, and that portions of the CAPRIN-1 proteins are specifically expressed on the surface of such cancer cells. Further, the present inventors have now found that antibodies against the CAPRIN-1 portions expressed on cancer cell surfaces can damage (or impair) cancer cells expressing CAPRIN-1. These findings have led to the completion of the present invention.

Therefore, the present invention has characteristics as described below.

The present invention provides a pharmaceutical composition for treatment and/or prevention of a cancer, which comprises, as an active ingredient, an antibody or a fragment thereof having an immunological reactivity with a CAPRIN-1 protein having an amino acid sequence shown in any one of the even numbered SEQ ID NOS: 2 to 30 or an amino acid sequence having 80% or more, preferably 85% or more, more preferably 90% or more, and further preferably 95% or more sequence identity with the amino acid sequence of any of the even-numbered SEQ ID NOS: 2 to 30, or with a fragment of the CAPRIN-1 protein comprising 7 or more consecutive amino acids.

In one embodiment of the present invention, the cancer is breast cancer, brain tumor, leukemia, lymphoma, lung cancer, esophageal cancer, colon cancer, gastric (or stomach) cancer, or kidney cancer.

In another embodiment of the present invention, the antibody is a monoclonal or polyclonal antibody.

In another embodiment of the present invention, the antibody is a human antibody, a humanized antibody, a chimeric antibody, a single-chain antibody, or a bispecific antibody.

In another embodiment of the present invention, the antibody is an antibody having an immunological reactivity with a polypeptide having the amino acid sequence shown in SEQ ID NO: 37 or SEQ ID NO: 136 or an amino acid sequence having 80% or more, preferably 85% or more, more preferably 90% or more, and further preferably 95% or more sequence identity with the amino acid sequence, or with a fragment of the polypeptide.

In another embodiment of the present invention, in the pharmaceutical composition for treatment and/or prevention of a cancer comprising the antibody as an active ingredient, the above antibody is any one of the antibodies (a) to (k) described below and has an immunological reactivity with a CAPRIN-1 protein.

(a) An antibody comprising a heavy-chain variable region comprising the sequences shown in SEQ ID NOS: 40, 41, and 42 and a light-chain variable region comprising the sequences shown in SEQ ID NOS: 44, 45, and 46.

(b) An antibody comprising a heavy-chain variable region comprising the sequences shown in SEQ ID NOS: 40, 41, and 42 and a light-chain variable region comprising the sequences shown in SEQ ID NOS: 50, 51, and 52.

(c) An antibody comprising a heavy-chain variable region comprising the sequences shown in SEQ ID NOS: 40, 41, and 42 and a light-chain variable region comprising the sequences shown in SEQ ID NOS: 55, 56, and 57.

(d) An antibody comprising a heavy-chain variable region comprising the sequences shown in SEQ ID NOS: 40, 41, and 42 and a light-chain variable region comprising the sequences shown in SEQ ID NOS: 60, 61, and 62.

(e) An antibody comprising a heavy-chain variable region comprising the sequences shown in SEQ ID NOS: 40, 41, and 42 and a light-chain variable region comprising the sequences shown in SEQ ID NOS: 65, 66, and 67.

(f) An antibody comprising a heavy-chain variable region comprising the sequences shown in SEQ ID NOS: 70, 71, and 72 and a light-chain variable region comprising the sequences shown in SEQ ID NOS: 74, 75, and 76.

(g) An antibody comprising a heavy-chain variable region comprising the sequences shown in SEQ ID NOS: 80, 81, and 82 and a light-chain variable region comprising the sequences shown in SEQ ID NOS: 84, 85, and 86.

(h) An antibody comprising a heavy-chain variable region comprising the sequences shown in SEQ ID NOS: 90, 91, and 92 and a light-chain variable region comprising the sequences shown in SEQ ID NOS: 94, 95, and 96.

(i) An antibody comprising a heavy-chain variable region comprising the sequences shown in SEQ ID NOS: 100, 101, and 102 and a light-chain variable region comprising the sequences shown in SEQ ID NOS: 104, 105, and 106.

(j) An antibody comprising a heavy-chain variable region comprising the sequences shown in SEQ ID NOS: 110, 111, and 112 and a light-chain variable region comprising the sequences shown in SEQ ID NOS: 114, 115, and 116.

(k) An antibody comprising a heavy-chain variable region comprising the sequences shown in SEQ ID NOS: 120, 121, and 122 and a light-chain variable region comprising the sequences shown in SEQ ID NOS: 124, 125, and 126.

### Effects of the Invention

Antibodies against CAPRIN-1 used in the present invention damage (or impair) cancer cells. Therefore, such antibodies against CAPRIN-1 are useful for treatment or prevention of cancers.

### BRIEF DESCRIPTION OF THE DRAWINGS

FIG. 1 shows expression patterns of genes encoding CAPRIN-1 proteins in normal tissues and tumor cell lines. In this Fig., reference no. 1 shows the expression pattern of each CAPRIN-1 coding gene, and reference no. 2 shows the expression pattern of GAPDH gene.

FIG. 2 shows the cytotoxic activity of an antibody to CAPRIN-1 (or anti-CAPRIN-1 antibody) against the breast cancer cell line expressing CAPRIN-1 gene (T47D). In this Fig., reference no. 3 shows the activity after addition of the anti-CAPRIN-1 antibody, reference no. 4 shows the activity after addition of control antibody, and reference no. 5 shows the activity in the absence of any antibody.

FIG. 3 shows the cytotoxic activity of an antibody to CAPRIN-1 (or anti-CAPRIN-1 antibody) against the breast cancer cell line expressing CAPRIN-1 gene (MDA-MB-157). In this Fig., reference no. 6 show the activity after addition of the anti-CAPRIN-1 antibody, reference no. 7 shows the activity after addition of control antibody and reference no. 8 shows the activity in the absence of any antibody.

FIG. 4 shows the cytotoxicity against the breast cancer MDA-MB-157 cell line expressing CAPRIN-1, wherein the cytotoxicity is exhibited by the monoclonal antibodies to CAPRIN-1 (i.e., the monoclonal antibodies #1 to #11), which are reactive with the surface of the cancer cell. Specifically, this Fig. shows the activity levels after addition of the #1 monoclonal antibody to CAPRIN-1 (reference no. 9), the #2 monoclonal antibody to CAPRIN-1 (reference no. 10), the #3 monoclonal antibody to CAPRIN-1 (reference no. 11), the #4 monoclonal antibody to CAPRIN-1 (reference no. 12), the #5 monoclonal antibody to CAPRIN-1 (reference no. 13), the #6 monoclonal antibody to CAPRIN-1 (reference no. 14), the #7 monoclonal antibody to CAPRIN-1 (reference no. 15), the #8 monoclonal antibody to CAPRIN-1 (reference no. 16), the #9 monoclonal antibody to CAPRIN-1 (reference no. 17), the #10 monoclonal antibody to CAPRIN-1 (reference no. 18), and the #11 monoclonal antibody to CAPRIN-1 (reference no. 19), the activity level after addition of a monoclonal antibody reactive with the CAPRIN-1 protein itself but not with the surface of the cancer cell (reference no. 20), and the activity level after addition of PBS instead of each antibody (reference no. 21).

FIGS. 5a to 5c show the antitumor effect of the monoclonal antibodies to CAPRIN-1 (i.e., the monoclonal antibodies #1 to #11) reactive with the surface of a cancer cell, on Balb/c mice into which the mouse carcinoma CT26 cell line expressing CAPRIN-1 was transplanted. These Figs. show the mouse tumor sizes after administration of the #1 monoclonal antibody to CAPRIN-1 (reference no. 22), the #2 monoclonal antibody to CAPRIN-1 (reference no. 23), the #3 monoclonal antibody to CAPRIN-1 (reference no. 24), the #4 monoclonal antibody to CAPRIN-1 (reference no. 25), the #5 monoclonal antibody to CAPRIN-1 (reference no. 26), the #6 monoclonal antibody to CAPRIN-1 (reference no. 27), the #7 monoclonal antibody to CAPRIN-1 (reference no. 28), the #8 monoclonal antibody to CAPRIN-1 (reference no. 29), the #9 monoclonal antibody to CAPRIN-1 (reference no. 30), the #10 monoclonal antibody to CAPRIN-1 (reference no. 31), and the #11 monoclonal antibody to CAPRIN-1 (reference no. 32), the mouse tumor size after administration of a monoclonal antibody reactive with a CAPRIN-1 protein itself but not with the surface of the cancer cell (reference no. 33), and the mouse tumor size after administration of PBS instead of each antibody (reference no. 34).

FIGS. 6a to 6c show the antitumor effect of monoclonal antibodies to CAPRIN-1 (i.e., the monoclonal antibodies #1 to #11) reactive with the surface of a cancer cell, on Balb/c mice into which the mouse carcinoma N1E cell line expressing CAPRIN-1 was transplanted. These Figs. show the mouse tumor sizes after administration of the #1 monoclonal antibody to CAPRIN-1 (reference no. 35), the #2 monoclonal antibody to CAPRIN-1 (reference no. 36), the #3 monoclonal antibody to CAPRIN-1 (reference no. 37), the #4 monoclonal antibody to CAPRIN-1 (reference no. 38), the #5 monoclonal antibody to CAPRIN-1 (reference no. 39), the #6 monoclonal antibody to CAPRIN-1 (reference no. 40), the #7 monoclonal antibody against CAPRIN-1 (reference no. 41), the #8 monoclonal antibody against CAPRIN-1 (reference no. 42), the #9 monoclonal antibody against CAPRIN-1 (reference no. 43), the #10 monoclonal antibody to CAPRIN-1 (reference no. 44), and the #11 monoclonal antibody against CAPRIN-1 (reference no. 45), the mouse tumor size after administration of a monoclonal antibody reactive with a CAPRIN-1 protein itself but not with the surface of the cancer cell (reference no. 46), and the mouse tumor size after administration of PBS instead of each antibody (reference no. 47).

### MODE FOR CARRYING OUT THE INVENTION

As described below, the antitumor activity of antibodies to the polypeptide shown in any one of the even-numbered SEQ ID NOS: 2 to 30 used in the present invention can be evaluated by examining in vivo the inhibition of tumor growth in a tumor-bearing animal, or by examining in vitro whether or not immunocyte- or complement-mediated cytotoxic activity against tumor cells expressing the polypeptide is exhibited.

In addition, the nucleotide sequences of polynucleotides encoding the proteins consisting of the amino acid sequences shown in the even-numbered SEQ ID NOS: 2 to 30 (i.e., SEQ ID NOS: 2, 4, 6 . . . 28, and 30) are shown in the odd-numbered SEQ ID NOS: 1 to 29 (i.e., SEQ ID NOS: 1, 3, 5 . . . 27, and 29), respectively.

The amino acid sequences shown in SEQ ID NOS: 6, 8, 10, 12 and 14 in the Sequence Listing disclosed according to the present invention are the amino acid sequences of the CAPRIN-1 proteins, which were isolated, by the SEREX method using canine testis tissue-derived cDNA libraries and sera from dogs with breast cancer, as polypeptides capable of binding to antibodies specifically existing in the sera from tumor-bearing dogs; the amino acid sequences shown in SEQ ID NOS: 2 and 4 are the amino acid sequences of the CAPRIN-1 proteins isolated as human homologs of said dog polypeptides; the amino acid sequence shown in SEQ ID NO: 16 is the amino acid sequence of the CAPRIN-1 protein isolated as a bovine homolog of said dog polypeptide; the amino acid sequence shown in SEQ ID NO: 18 is the amino acid sequence of the CAPRIN-1 protein isolated as an equine homolog of said dog polypeptide; the amino acid sequences shown in (even-numbered) SEQ ID NOS: 20 to 28 are the amino acid sequences of the CAPRIN-1 proteins isolated as murine homologs of said dog polypeptides; and the amino acid sequence shown in SEQ ID NO: 30 is the amino acid sequence of the CAPRIN-1 protein isolated as a chicken homolog of said dog polypeptide (see Example 1 described below). CAPRIN-1 is known to be expressed when activation or cell division of normal cells in resting phase takes place.

It was known that CAPRIN-1 was not expressed on the surface of cells. However, as a result of examination in connection with the present invention, it has been now revealed that certain portions of CAPRIN-1 protein are expressed on the surfaces of various cancer cells. According to the present invention, an antibody that binds to a portion within CAPRIN-1 protein expressed on cancer cell surfaces is preferably used. Examples of the partial peptides within CAPRIN-1 protein expressed on cancer cell surfaces include polypeptides consisting of a sequence of 7 or more consecutive amino acids in the region of the amino acid residue Nos. (or the amino acids (aa)) 50-98 or the amino acid residue Nos. (aa) 233-305 in an amino acid sequence shown in any one of the even-numbered SEQ ID NOS: 2 to 30, excluding SEQ ID NOS: 6 and 18, in the Sequence Listing. Specific examples thereof include the amino acid sequence shown in SEQ ID NO: 37 or 136 (preferably, the region of the amino acid sequence shown in SEQ ID NO: 137 or 138 in the amino acid sequence shown in SEQ ID NO: 136), or an amino acid sequence having 80% or more, preferably 85% or more, more preferably 90% or more, and further preferably 95% or more sequence identity with said amino acid sequences. Antibodies of the present invention include all antibodies capable of binding to the above peptides and having antitumor activity.

The antibodies to CAPRIN-1 usable in the present invention as described above may be any types thereof, as long as they can exhibit antitumor activity. Examples thereof include monoclonal antibodies, polyclonal antibodies, synthetic antibodies, multispecific antibodies, human antibodies, humanized antibodies, chimeric antibodies, single-chain antibodies (scFV), and fragments thereof such as Fab and F(ab′)₂. These antibodies and fragments thereof can be prepared by methods known to persons skilled in the art. In the present invention, antibodies capable of specifically binding to a CAPRIN-1 protein are desirable. Such antibodies are preferably monoclonal antibodies; however, as long as homogenous antibodies can be stably produced, polyclonal antibodies may also be used. In addition, if the subject is a human, a human antibody or a humanized antibody is desirable in order to avoid or inhibit the immunorejection.

The word “specifically binding to a CAPRIN-1 protein” as used herein means that an antibody of interest specifically binds to the CAPRIN-1 protein and does not substantially bind to other proteins.

As described below, the antitumor activity of an antibody used in the present invention can be evaluated by examining in vivo the inhibition of tumor growth in a tumor-bearing animal, or examining in vitro whether or not the immunocyte- or complement-mediated cytotoxic activity against tumor cells expressing the polypeptide is exhibited.

Moreover, the subjects in need of treatment and/or prevention of cancer according to the present invention are mammals such as human, pet animals, livestock animals, or sport animals. The preferred subject is a human.

Production of antigens, production of antibodies, and pharmaceutical compositions, related to the present invention, will be explained below.

### <Production of Antigens Used for Antibody Production>

Proteins or fragments thereof used as sensitizing antigens for obtaining antibodies to CAPRIN-1 used in the present invention are not limited in terms of their origins such as animals including, for example, humans, canines, bovines, horses, mice, rats, and chickens. However, such proteins or fragments thereof are preferably selected in view of compatibility with parent cells used for cell fusion. Mammal-derived proteins are generally preferable and human-derived proteins are particularly preferable. For instance, if the CAPRIN-1 is human CAPRIN-1, a human CAPRIN-1 protein, a partial peptide thereof, or cells capable of expressing human CAPRIN-1 can be used.

Nucleotide sequences and amino acid sequences of human CAPRIN-1 and homologs thereof can be obtained by, for example, accessing GenBank (NCBI, USA) and using the BLAST or FASTA algorithm (Karlin and Altschul, Proc. Natl. Acad. Sci. USA, 90:5873-5877, 1993; Altschul et al., Nucleic Acids Res. 25:3389-3402, 1997).

According to the present invention, when the nucleotide sequence (SEQ ID NO: 1 or 3) or the amino acid sequence (SEQ ID NO: 2 or 4) of human CAPRIN-1 is used as a base sequence, targets are nucleic acids or proteins each consisting of a sequence having 70% to 100%, preferably 80% to 100%, more preferably 90% to 100%, and further preferably 95% to 100% (e.g., 97% to 100%, 98% to 100%, 99% to 100%, or 99.5% to 100%) sequence identity with the nucleotide sequence or amino acid sequence of the ORF or mature portion of the base nucleotide sequence or amino acid sequence. The term “% sequence identity” as used herein means a percentage (%) of the number of identical amino acids (or nucleotides) to the total number of amino acids (or nucleotides) in the case that two sequences are aligned such that maximum similarity can be achieved with or without introduction of gaps.

Fragments of a CAPRIN-1 protein have lengths ranging from the amino acid length of an epitope (or an antigenic determinant), which is the smallest unit of an antigen recognized by an antibody, to less than the full-length of the protein. The epitope refers to a polypeptide fragment having antigenicity or immunogenicity in mammals and preferably in humans. The smallest unit of polypeptide fragment consists of approximately 7 to 12 amino acids, and for example, 8 to 11 amino acids. A specific example thereof is the amino acid sequence shown in SEQ ID NO: 37, SEQ ID NO: 137, or SEQ ID NO: 138, or an amino acid sequence having 80% or more, preferably 85% or more, more preferably 90% or more, and further preferably 95% or more sequence identity with said amino acid sequence.

Polypeptides comprising the aforementioned human CAPRIN-1 protein and partial peptides thereof can be synthesized according to chemical synthesis methods such as the Fmoc method (fluorenylmethyloxycarbonyl method) or the tBoc method (t-butyloxycarbonyl method) (the Japanese Biochemical Society (ed.), “Biochemical Experimentation Course (Seikagaku Jikken Koza) 1,” Protein Chemistry IV, Chemical Modification and Peptide Synthesis, Kagaku-dojin Publishing Company, Inc. (Japan), 1981). Also, they can be synthesized by general methods using a variety of commercially available peptide synthesizers. In addition, polypeptides of interest can be obtained by preparing polynucleotides encoding the above polypeptides using known gene engineering methods (Sambrook et al., Molecular Cloning, 2nd edition, Current Protocols in Molecular Biology (1989), Cold Spring Harbor Laboratory Press; Ausubel et al., Short Protocols in Molecular Biology, 3rd edition, A Compendium of Methods from Current Protocols in Molecular Biology (1995), John Wiley & Sons, etc.), incorporating each of the polynucleotides into an expression vector and introducing the vector into a host cell, thereby allowing the host cell to produce the polypeptide. By such a way, the desired polypeptides can be obtained.

Polynucleotides encoding the aforementioned polypeptides can be readily prepared by known gene engineering techniques or general methods using commercially available nucleic acid synthesizers. For example, DNA comprising the nucleotide sequence shown in SEQ ID NO: 1 can be prepared by PCR using a human chromosome DNA or cDNA library as a template and a pair of primers designed to enable the amplification of the nucleotide sequence shown in SEQ ID NO: 1. PCR conditions can be appropriately determined. For example, such conditions may comprise conducting 30 cycles of the reaction steps (as one cycle) consisting of: 94° C., 30 seconds (denaturation); 55° C., 30 seconds to 1 minute (annealing); and 72° C., r 2 minutes (elongation) using a thermostable DNA polymerase (e.g., Taq polymerase) and a Mg²⁺-containing PCR buffer, followed by reaction at 72° C. for 7 minutes after completion of the 30 cycles. However, the present invention is not limited to the above-exemplified PCR conditions. PCR techniques and conditions are described in, for example, Ausubel et al., Short Protocols in Molecular Biology, 3rd edition, A Compendium of Methods from Current Protocols in Molecular Biology (1995), John Wiley & Sons (Chapter 15, in particular).

In addition, desired DNA can be isolated by preparing appropriate probes and primers based on information about the nucleotide and amino acid sequences shown in SEQ ID NOS: 1 to 30 in the Sequence Listing described herein, and screening a human cDNA library or the like with the use of such probes and primers. Preferably, such cDNA library is produced from a cell, organ, or tissue in which the protein with any one of the even-numbered SEQ ID NOS: 2 to 30 is expressed. Examples of the cell or tissue include cells or tissues from testis and cancers or tumors, such as leukemia, breast cancer, lymphoma, brain tumor, lung cancer, and colon cancer. Operations such as preparation of probes or primers, construction of cDNA libraries, screening of cDNA libraries, and cloning of genes of interest, as described above, are known to persons skilled in the art, and they can be carried out according to, for example, the methods described in Sambrook et al., Molecular Cloning, the 2nd edition, Current Protocols in Molecular Biology (1989) and Ausbel et al. (ibid.). DNAs encoding human CAPRIN-1 protein and partial peptides thereof can be obtained from the thus obtained DNAs.

The above-described host cells may be any cells, as long as they can express the above-described polypeptides. An example of prokaryotic host cell includes, but is not limited to, Escherichia coli. Examples of eukaryotic host cells include, but are not limited to, mammalian cells such as monkey kidney cell (COS 1), Chinese hamster ovary cell (CHO), human embryonic kidney cell line (HEK293), and mouse embryonic skin cell line (NIH3T3), yeast cells such as budding yeast and dividing yeast cells, silkworm cells, and Xenopus egg cells.

When prokaryotic cells are used as host cells, an expression vector having an origin replicable in prokaryotic cells, a promoter, a ribosome-binding site, a multicloning site, a terminator, a drug resistance gene, an auxotrophic complementary gene, or the like can be used. As expression vectors for Escherichia coli, pUC vectors, pBluescriptII, pET expression systems, pGEX expression systems, and the like can be exemplified. A DNA encoding the above polypeptide is incorporated into such an expression vector, a prokaryotic host cell is transformed with the vector, and then the thus obtained transformed cell is cultured, so that the polypeptide encoded by the DNA can be expressed in the prokaryotic host cell. At this time, the polypeptide can also be expressed as a fusion protein with another protein.

When eukaryotic cells are used as host cells, expression vectors for eukaryotic cells having a promoter, a splicing region, a poly(A) addition site, or the like can be used. Examples of such expression vectors include pKA1, pCDM8, pSVK3, pMSG, pSVL, pBK-CMV, pBK-RSV, EBV vector, pRS, pcDNA3, and pYES2. By similar procedures to those mentioned above, a DNA encoding the aforementioned polypeptide is incorporated into such an expression vector, an eukaryotic host cell is transformed with the vector, and then the thus obtained transformed cell is cultured, so that the polypeptide encoded by the above DNA can be expressed in the eukaryotic host cell. When pIND/V5-His, pFLAG-CMV-2, pEGFP-N1, pEGFP-C1, or the like is used as an expression vector, the above polypeptide may be expressed as a fusion protein with a tag, such as His tag (e.g., (His)₆ to (His)₁₀), FLAG tag, myc tag, HA tag, or GFP.

For introduction of an expression vector into a host cell, well known methods can be employed, such as electroporation, a calcium phosphate method, a liposome method, a DEAE dextran method, microinjection, viral infection, lipofection, and binding with a cell-membrane-permeable peptide.

Isolation and purification of a polypeptide of interest from host cells can be performed using known isolation techniques in combination. Examples of such known techniques include, but are not limited to, treatment using a denaturing agent such as urea or a surfactant, ultrasonication, enzymatic digestion, salting-out, solvent fractionation and precipitation, dialysis, centrifugation, ultrafiltration, gel filtration, SDS-PAGE, isoelectric focusing electrophoresis, ion exchange chromatography, hydrophobic chromatography, affinity chromatography, and reverse phase chromatography.

### <Structure of Antibody>

In general, antibodies are heteromultimeric glycoproteins each comprising at least two heavy chains and two light chains. Meanwhile, antibodies except for IgM are heterotetrameric glycoproteins (approximately 150 kDa) each comprising two identical light (L) chains and two identical heavy (H) chains. Typically, each light chain is connected to a heavy chain via a single covalent disulfide bond. However, the number of disulfide bonds between heavy chains varies among different immunoglobulin isotypes. Each of heavy chain and light chain also has an intrachain disulfide bond(s). Each heavy chain has a variable domain (VH region) at one end thereof, to which some constant regions are bound in series. Each light chain has a variable domain (VL region) at one end thereof and has a single constant region at the opposite end thereof. The constant region of a light chain is aligned with the first constant region of a heavy chain and the light-chain variable domain is aligned with the heavy-chain variable domain. A specific region of an antibody variable domain, which is called “complementarity determining region (CDR),” exhibits specific variability so as to impart binding specificity to an antibody. A relatively conserved portion in a variable region is called a “framework region (FR).” A complete heavy-chain or light-chain variable domain comprises 4 FRs connected to each other via 3 CDRs. Such CDRs are called “CDRH1,” “CDRH2,” and “CDRH3,” respectively, in such order from the N-terminus in a heavy chain. Similarly, for a light chain, they are called “CDRL1,” “CDRL2,” and “CDRL3,” respectively. CDRH3 plays the most important role in terms of antibody-antigen binding specificity. In addition, CDRs in each chain are retained by FR regions in the state that they are close to each other, and they contribute to the formation of antibody-antigen binding sites with CDRs in a corresponding chain. Constant regions do not directly contribute to antibody-antigen binding. However, they exhibit various effector functions such as association with antibody-dependent cell-mediated cytotoxicity (ADCC), phagocytosis through binding to an Fcγ receptor, half-life/clearance rate via an neonatal Fc receptor (FcRn), and complement-dependent cytotoxicity (CDC) via a C1q component in the complement cascade.

### <Antibody Production>

The term “anti-CAPRIN-1 antibody” used in the present invention refers to an antibody having an immunological reactivity with a full-length CAPRIN-1 protein or a fragment thereof.

The term “immunological reactivity” used herein indicates the characteristics of an antibody binding in vivo to a CAPRIN-1 antigen. The tumor-damaging function (e.g., death, inhibition, or regression) can be expressed as a result of such binding. Specifically, any type of antibody may be used in the present invention as long as the antibody can bind to a CAPRIN-1 protein to damage a tumor or a cancer such as leukemia, lymphoma, breast cancer, brain tumor, lung cancer, esophageal cancer, gastric cancer, kidney cancer, or colon cancer.

Examples of such antibodies include monoclonal antibodies, polyclonal antibodies, synthetic antibodies, multispecific antibodies, human antibodies, humanized antibodies, chimeric antibodies, single-chain antibodies, and antibody fragments (e.g., Fab and F(ab′)₂). In addition, examples of arbitrary immunoglobulin classes of such antibodies include IgG, IgE, IgM, IgA, IgD, and IgY, and examples of arbitrary immunoglobulin subclasses include IgG1, IgG2, IgG3, IgG4, IgA1, and IgA2.

Antibodies may be further modified via acetylation, formylation, amidation, phosphorylation, or pegylation (PEG), in addition to glycosylation.

Production examples for a variety of antibodies are described below.

In a case in which an antibody of interest is a monoclonal antibody, a breast cancer SK-BR-3 cell line expressing CAPRIN-1 or the like is administered to mice for immunization, followed by extraction of spleens from the mice. Cells are separated from each spleen and then are fused with mouse myeloma cells. Clones capable of producing an antibody having cancer cell growth inhibition action are selected from the obtained fusion cells (hybridomas). A monoclonal antibody-producing hybridoma having cancer cell growth inhibition action is isolated and cultured. An antibody of interest can be prepared via purification from the culture supernatant by a general affinity purification method.

Also, a monoclonal antibody-producing hybridoma can be produced in a manner described below, for example. First, an animal is immunized with a sensitizing antigen by a known method. In a general method, immunization is carried out by intraperitoneally or subcutaneously injecting a sensitizing antigen into a mammal. Specifically, a sensitizing antigen is diluted with or suspended in PBS (Phosphate-Buffered Saline), physiological saline, or the like to an appropriate resultant amount. If desired, an appropriate amount of a conventional adjuvant (e.g., Freund's complete adjuvant) is mixed therewith. After emulsification takes place, the resultant is administered to a mammal several times every 4 to 21 days. In addition, an adequate carrier can be used for immunization with a sensitizing antigen.

As described above, after immunization of a mammal and confirmation of an increase to a desired antibody level in serum, immunocytes are collected from the mammal and subjected to cell fusion. Particularly preferable examples of immunocytes are splenocytes.

Mammalian myeloma cells are used as relevant parent cells subjected to fusion with the above immunocytes. For such myeloma cells, the following various examples of known cell lines are preferably used: P3U1 (P3-X63Ag8U1), P3 (P3x63Ag8.653) (J. Immunol. (1979) 123, 1548-1550), P3x63Ag8U.1 (Current Topics in Microbiology and Immunology (1978) 81, 1-7), NS-1 (Kohler. G. and Milstein, C. Eur. J. Immunol. (1976). 6, 511-519), MPC-11 (Margulies. D. H. et al., Cell (1976) 8, 405-415), SP2/0 (Shulman, M. et al., Nature (1978) 276, 269-270), FO (de St. Groth, S. F. et al., J. Immunol. Methods (1980) 35, 1-21), S194 (Trowbridge, I. S. J. Exp. Med. (1978) 148, 313-323), and R210 (Galfre, G. et al., Nature (1979) 277, 131-133).

Basically, cell fusion of immunocytes and myeloma cells described above can be carried out according to a known method such as the method of Kohler and Milstein et al. (Kohler, G. and Milstein, C. Methods Enzymol. (1981) 73, 3-46).

More specifically, cell fusion described above is carried out in the presence of a cell fusion promoter in a conventional nutrients-containing culture solution, for example. Examples of a fusion promoter to be used include polyethylene glycol (PEG) and Sendai virus (HVJ: hemagglutinating virus of Japan). If desired, an adjuvant such as dimethylsulfoxide may be further added for improvement of fusion efficiency.

The proportion of immunocytes used to that of myeloma cells used can be arbitrarily determined. For example, the ratio of immunocytes to myeloma cells is preferably 1:1 to 10:1. Examples of a culture solution that can be used for cell fusion described above include an RPMI1640 culture solution and an MEM culture solution adequate for growth of the above myeloma cell lines as well as other conventional culture solutions used for this kind of cell culture. Further, a serum replacement such as fetal calf serum (FCS) can be used in combination therewith.

For cell fusion, the above immunocytes and myeloma cells are sufficiently mixed at predetermined amounts in the culture solution. A PEG solution (e.g., average molecular weight: approximately 1000 to 6000) that has been previously heated to approximately 37° C. is added thereto at a concentration of generally 30% to 60% (w/v), followed by mixing. This results in formation of hybridomas of interest. Subsequently, operational steps of sequential addition of an appropriate culture solution and removal of the supernatant via centrifugation are repeatedly carried out to remove cell fusion agent(s) and the like that are not preferable for the growth of hybridomas.

The thus obtained hybridomas are cultured in a conventional selection culture solution such as an HAT culture solution (a culture solution comprising hypoxanthine, aminopterin, and thymidine) for selection. Culture in such an HAT culture solution is continuously carried out for a sufficient time period (generally several days to several weeks) for death of cells (non-fused cells) other than hybridomas of interest. Next, a conventional limiting dilution method is employed to screen for hybridomas producing antibodies of interest and to carry out single cloning.

Further, it is also possible to obtain human antibody-producing hybridomas having desired activity (e.g., cell growth inhibition activity) in the following manner, as well as to obtain the above hybridomas via immunization of non-human animals with antigens. Human lymphocytes (e.g., human lymphocytes infected with EB virus) are sensitized in vitro with a protein, protein-expressing cells, or a lysate thereof and sensitized lymphocytes are fused with human-derived myeloma cells having the ability to permanently divide (e.g., U266) (registration no. TIB196).

Monoclonal antibody-producing hybridomas produced as above can be subcultured in a conventional culture solution. In addition, they can be preserved in liquid nitrogen for a long period of time.

Specifically, immunization is carried out using a desired antigen or cells expressing a desired antigen as sensitizing antigen(s) according to a conventional immunization method. The obtained immunocytes are fused with known parent cells by a conventional cell fusion method. Then, monoclonal antibody-producing cells (hybridomas) are screened for by a conventional screening method. Thus, antibody production can be carried out.

Other examples of antibodies that can be used in the present invention include polyclonal antibodies. For example, polyclonal antibodies can be used in a manner described below.

Serum is obtained by immunizing small animals such as mice, human antibody-producing mice, or rabbits with a naturally occurring CAPRIN-1 protein, a recombinant CAPRIN-1 protein that has been expressed as a protein fused with GST or the like in a microorganism such as Escherichia coli, or a partial peptide thereof. The serum is purified via ammonium sulfate precipitation, protein A/protein G column chromatography, DEAE ion-exchange chromatography, affinity column chromatography with a column to which a CAPRIN-1 protein or a synthetic peptide is coupled, or a similar technique for preparation of polyclonal antibodies. In the Examples described below, a rabbit polyclonal antibody was produced, and antitumor effects thereof were confirmed, such antibody being against a partial peptide (with the sequence shown in SEQ ID NO: 37) of a domain in a CAPRIN-1 protein amino acid sequence that is expressed on cancer cell surfaces.

A known human antibody-producing mouse used herein is, for example, a KM Mouse (Kirin Pharma/Medarex) or a XenoMouse (Amgen) (e.g., WO02/43478 and WO02/092812). When such mice are immunized with CAPRIN-1 proteins or fragments thereof, complete human polyclonal antibodies can be obtained from blood. In addition, human monoclonal antibodies can be produced by a method of fusing splenocytes collected from immunized mice with myeloma cells.

Antigen preparation can be carried out in accordance with a method such as a method using animal cells (JP Patent Publication (Kohyo) No. 2007-530068) or a method using a baculovirus (e.g., WO98/46777). If the immunogenicity of an antigen is low, an antigen is bound to a macromolecule having immunogenicity, such as albumin. Then, the antigen can be used for immunization.

Further, it is possible to use a gene recombinant antibody produced by cloning an antibody gene from a hybridoma, incorporating the clone into an adequate vector, introducing the vector into a host, and using a gene recombinant technique. (See, for example, Carl, A. K. Borrebaeck, James, W. Larrick, THERAPEUTIC MONOCLONAL ANTIBODIES, Published in the United Kingdom by MACMILLAN PUBLISHERS LTD, 1990.) Specifically, cDNA of a variable region (V region) of an antibody is synthesized from mRNA of a hybridoma with the use of a reverse transcriptase. After DNA encoding a V region of an antibody of interest is obtained, such DNA is ligated to desired DNA encoding an antibody constant region (C region). The resultant is incorporated into an expression vector. Alternatively, DNA encoding an antibody V region may be incorporated into an expression vector comprising DNA of an antibody C region. Such DNA is incorporated into an expression vector in a manner such that it is expressed under control of an expression control region such as an enhancer or a promoter. Next, host cells are transformed with such expression vector, thereby allowing the antibody to be expressed.

Anti-CAPRIN-1 antibodies of the present invention are preferably monoclonal antibodies. However, they may be polyclonal antibodies, gene-modified antibodies (such as chimeric antibodies and humanized antibodies), and the like.

Monoclonal antibodies include human monoclonal antibodies and non-human animal monoclonal antibodies (e.g., mouse monoclonal antibodies, rat monoclonal antibodies, rabbit monoclonal antibodies, and chicken monoclonal antibodies). Monoclonal antibodies can be produced by culturing hybridomas obtained via fusion of myeloma cells and splenocytes from non-human mammals (e.g., mice or human antibody-producing mice) immunized with CAPRIN-1 proteins. In the Examples described below, mouse monoclonal antibodies were produced and antitumor effects thereof were confirmed. Such a monoclonal antibody comprises a heavy-chain variable (VH) region having the amino acid sequence shown in SEQ ID NO: 43, SEQ ID NO: 73, SEQ ID NO: 83, SEQ ID NO: 93, SEQ ID NO: 103, SEQ ID NO: 113, or SEQ ID NO: 123 and a light-chain variable (VL) region having the amino acid sequence shown in SEQ ID NO: 47, SEQ ID NO: 53, SEQ ID NO: 58, SEQ ID NO: 63, SEQ ID NO: 68, SEQ ID NO: 77, SEQ ID NO: 87, SEQ ID NO: 97, SEQ ID NO: 107, SEQ ID NO: 117, or SEQ ID NO: 127. Here, the VH region comprises: CDR1 represented by the amino acid sequence of SEQ ID NO: 40, SEQ ID NO: 70, SEQ ID NO: 80, SEQ ID NO: 90, SEQ ID NO: 100, SEQ ID NO: 110, or SEQ ID NO: 120; CDR2 represented by the amino acid sequence of SEQ ID NO: 41, SEQ ID NO: 71, SEQ ID NO: 81, SEQ ID NO: 91, SEQ ID NO: 101, SEQ ID NO: 111, or SEQ ID NO: 121; and CDR3 represented by the amino acid sequence of SEQ ID NO: 42, SEQ ID NO: 72, SEQ ID NO: 82, SEQ ID NO: 92, SEQ ID NO: 102, SEQ ID NO: 112, or SEQ ID NO: 122. The VL region comprises: CDR1 represented by the amino acid sequence of SEQ ID NO: 44, SEQ ID NO: 50, SEQ ID NO: 55, SEQ ID NO: 60, SEQ ID NO: 65, SEQ ID NO: 74, SEQ ID NO: 84, SEQ ID NO: 94, SEQ ID NO: 104, SEQ ID NO: 114, or SEQ ID NO: 124; CDR2 represented by the amino acid sequence of SEQ ID NO: 45, SEQ ID NO: 51, SEQ ID NO: 56, SEQ ID NO: 61, SEQ ID NO: 66, SEQ ID NO: 75, SEQ ID NO: 85, SEQ ID NO: 95, SEQ ID NO: 105, SEQ ID NO: 115, or SEQ ID NO: 125; and CDR3 represented by the amino acid sequence of SEQ ID NO: 46, SEQ ID NO: 52, SEQ ID NO: 57, SEQ ID NO: 62, SEQ ID NO: 67, SEQ ID NO: 76, SEQ ID NO: 86, SEQ ID NO: 96, SEQ ID NO: 106, SEQ ID NO: 116, or SEQ ID NO: 126.

A chimeric antibody is an antibody produced by combining sequences from different animals. An example thereof is an antibody consisting of mouse antibody heavy-chain and light-chain variable regions and human antibody heavy-chain and light-chain constant regions. Such a chimeric antibody can be produced by a known method. For example, it can be obtained by ligating DNA encoding an antibody V region to DNA encoding a human antibody C region, incorporating the resultant into an expression vector, and introducing the vector into a host for antibody production.

Polyclonal antibodies include antibodies obtained by immunizing human antibody-producing animals (e.g., mice) with CAPRIN-1 proteins.

A humanized antibody is a modified antibody, and it is sometimes referred to as a “reshaped human antibody.” It is known that a humanized antibody is constructed by transplanting CDRs of an immunized animal-derived antibody into complementarity determining regions of a human antibody. Also, a general gene recombinant technique therefor is known.

Specifically, a DNA sequence designed in a manner that allows mouse antibody CDRs to be ligated to human antibody framework regions (FRs) is synthesized by the PCR method using several oligonucleotides prepared in such a manner that the oligonucleotides have portions overlapping each other at one end of each thereof. A humanized antibody can be obtained by ligating the above obtained DNA to DNA encoding a human antibody constant region, incorporating the resultant into an expression vector, and introducing the vector into a host for antibody production (see EP-A-239400 and WO96/02576). Human antibody FRs ligated to each other via CDRs are selected on the assumption that complementarity determining regions can form a good antigen binding site. If necessary, amino acids in framework regions of an antibody variable region may be substituted in such a manner that complementarity determining regions in a reshaped human antibody form an appropriate antigen binding site (Sato K. et al., Cancer Research 1993, 53: 851-856). In addition, the framework regions may be substituted with framework regions from a different human antibody (see WO99/51743).

Human antibody framework regions ligated to each other via CDRs are selected on the assumption that complementarity determining regions can form good antigen binding sites. If necessary, amino acids in framework regions of an antibody variable region may be substituted in such a manner that complementarity determining regions in reshaped human antibody form an appropriate antigen binding sites (Sato K. et al., Cancer Research 1993, 53: 851-856).

After a chimeric antibody or a humanized antibody is produced, amino acids in a variable region (e.g., FR) or a constant region may be substituted, for example, with different amino acids.

Here, the amino acid substitution is a substitution of, for example, less than 15, less than 10, not more than 8, not more than 7, not more than 6, not more than 5, not more than 4, not more than 3, or not more than 2 amino acids, preferably 1 to 5 amino acids, and more preferably 1 or 2 amino acids. A substituted antibody should be functionally equivalent to an unsubstituted antibody. The substitution is preferably a conservative amino acid substitution, which is a substitution between amino acids having similar characteristics in terms of charge, side chains, polarity, aromaticity, and the like. For example, characteristically similar amino acids can be classified into the following types: basic amino acids (arginine, lysine, and histidine); acidic amino acids (aspartic acid and glutamic acid); uncharged polar amino acids (glycine, asparagine, glutamine, serine, threonine, cysteine, and tyrosine); nonpolar amino acids (leucine, isoleucine, alanine, valine, proline, phenylalanine, tryptophan, and methionine); branched-chain amino acids (threonine, valine, isoleucine); and aromatic amino acids (phenylalanine, tyrosine, tryptophan, and histidine).

An example of an antibody modifier is an antibody bound to a molecule such as polyethylene glycol (PEG). Regarding antibody modifiers of the present invention, substances that bind to an antibody are not limited. Such an antibody modifier can be obtained by chemically modifying an obtained antibody. A method of such modification has been already established in the field related to the present invention.

The expression “functionally equivalent” used herein indicates a situation in which an antibody of interest has biological or biochemical activity similar to that of an antibody of the present invention. Specifically, such antibody has a function of damaging tumors and causes essentially no rejection reaction when applied to humans. An example of such activity is cell growth inhibition activity or binding activity.

A known method for preparing a polypeptide functionally equivalent to a given polypeptide that is well known to persons skilled in the art is a method comprising introducing a mutation into a polypeptide. For instance, a person skilled in the art can adequately introduce a mutation into an antibody of the present invention using a site-specific mutagenesis method (Hashimoto-Gotoh, T. et al., (1995) Gene 152, 271-275; Zoller, M J., and Smith, M. (1983) Methods Enzymol. 100, 468-500; Kramer, W. et al., (1984) Nucleic Acids Res. 12, 9441-9456; Kramer, W. and Fritz, H J., (1987) Methods Enzymol. 154, 350-367; Kunkel, T A., (1985) Proc. Natl. Acad. Sci. USA. 82, 488-492; or Kunkel (1988) Methods Enzymol. 85, 2763-2766) or a similar method. Thus, an antibody functionally equivalent to the antibody of the present invention can be prepared.

An aforementioned antibody capable of recognizing an epitope of a CAPRIN-1 protein recognized by an anti-CAPRIN-1 antibody can be obtained by a method known to persons skilled in the art. For example, it can be obtained by: a method comprising determining an epitope of a CAPRIN-1 protein recognized by an anti-CAPRIN-1 antibody by a general method (e.g., epitope mapping) and producing an antibody using a polypeptide having an amino acid sequence contained in the epitope as an immunogen; or a method comprising determining an epitope of an antibody produced by a general method and selecting an antibody having an epitope identical to an epitope of an anti-CAPRIN-1 antibody. Here, the term “epitope” refers to a polypeptide fragment having antigenicity or immunogenicity in mammals and preferably in humans. The smallest unit thereof consists of approximately 7 to 12 amino acids and preferably 8 to 11 amino acids.

The affinity constant Ka (kₒₙ/k_{off}) of an antibody of the present invention is preferably at least 10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 10¹³ M⁻¹.

An antibody of the present invention can be conjugated with an antitumor agent. Binding between an antibody and an antitumor agent can be carried out via a spacer having a group reactive to an amino group, a carboxyl group, a hydroxy group, a thiol group, or the like (e.g., an imidyl succinate group, a formyl group, a 2-pyridyldithio group, a maleimidyl group, an alkoxycarbonyl group, or a hydroxy group).

Examples of antitumor agents include the following antitumor agents known in references or the like: paclitaxel, doxorubicin, daunorubicin, cyclophosphamide, methotrexate, 5-fluorouracil, thiotepa, busulfan, improsulfan, piposulfan, benzodopa, carboquone, meturedopa, uredopa, altretamine, triethylenemelamine, triethylenephosphoramide, triethilenethiophosphoramide, trimethylolomelamine, bullatacin, bullatacinone, camptothecin, bryostatin, callystatin, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin, aclacinomycin, actinomycin, authramycin, azaserine, bleomycin, cactinomycin, carabicin, carminomycin, carzinophilin, chromomycin, dactinomycin, detorbicin, 6-diazo-5-oxo-L-norleucine, ADRIAMYCIN, epirubicin, esorubicin, idarubicin, marcellomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, denopterin, pteropterin, trimetrexate, fludarabine, 6-mercaptopurine, thiamiprine, thioguanine, ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, lentinan, lonidamine, maytansine, ansamitocine, mitoguazone, mitoxantrone, mopidanmol, nitraerine, pentostatin, phenamet, pirarubicin, losoxantrone, podophyllinic acid, 2-ethylhydrazide, procarbazine, razoxane, rhizoxin, schizophyllan, spirogermanium, tenuazonic acid, triaziquone, roridine A, anguidine, urethane, vindesine, dacarbazine, mannomustine, mitobronitol, mitolactol, pipobroman, gacytosine, docetaxel, chlorambucil, gemcitabine, 6-thioguanine, mercaptopurine, cisplatin, oxaliplatin, carboplatin, vinblastine, etoposide, ifosfamide, mitoxantrone, vincristine, vinorelbine, novantrone, teniposide, edatrexate, daunomycin, aminopterin, xeloda, ibandronate, irinotecan, topoisomerase inhibitor, difluoromethylornithine (DMFO), retinoic acid, capecitabine, and pharmacologically acceptable salts or derivatives thereof.

Alternatively, it is also possible to bind a radioactive isotope such as ²¹¹At, ¹³¹I, ¹²⁵I, ⁹⁰Y, ¹⁸⁶Re, ¹⁸⁸Re, ¹⁵³Sm, ²¹²Bi, ³²P, ¹⁷⁵Lu, or ¹⁷⁶Lu known in references and the like to an antibody of the present invention. It is desirable for such radioactive isotopes to be effective for tumor treatment or diagnosis.

An antibody of the present invention is an antibody having an immunological reactivity with CAPRIN-1 or an antibody capable of specifically recognizing CAPRIN-1. Such an antibody should be an antibody having a structure that allows a subject animal to which the antibody is administered to completely or almost completely avoid a rejection reaction. If the subject animal is a human, examples of the above antibody include human antibodies, humanized antibodies, chimeric antibodies (e.g., human-mouse chimeric antibodies), single-chain antibodies, and bispecific antibodies. Such an antibody is a recombinant antibody having human antibody-derived heavy-chain and light-chain variable regions, a recombinant antibody having heavy-chain and light-chain variable regions each consisting of non-human animal antibody-derived complementarity determining regions (CDR1, CDR2, and CDR3) and human antibody-derived framework regions, or a recombinant antibody having non-human animal antibody-derived heavy-chain and light-chain variable regions and human antibody-derived heavy-chain and light-chain constant regions. The first two antibodies are preferable.

The above recombinant antibody can be produced in the manner described below. DNA encoding a monoclonal antibody against human CAPRIN-1 (e.g., a human monoclonal antibody, a mouse monoclonal antibody, a rat monoclonal antibody, a rabbit monoclonal antibody, or a chicken monoclonal antibody) is cloned from an antibody-producing cell such as a hybridoma. DNAs encoding a light-chain variable region and a heavy-chain variable region of the antibody are produced by an RT-PCR method or the like using the obtained clone as a template. Then, the sequences of a light-chain variable region and a heavy-chain variable region or the sequences of CDR1, CDR2, and CDR3 are determined by the Kabat EU numbering system (Kabat et al., Sequences of Proteins of Immunological Interest, 5th Ed. Public Health Service, National Institute of Health, Bethesda, Md. (1991)).

Further, such DNAs encoding variable regions or DNAs encoding CDRs are produced by a gene recombinant technique (Sambrook et al., Molecular Cloning A Laboratory Manual, Cold Spring Harbor Laboratory Press (1989)) or a DNA synthesizer. Here, the above human monoclonal antibody-producing hybridoma can be produced by immunizing a human antibody-producing animal (e.g., a mouse) with human CAPRIN-1 and fusing splenocytes from the spleen removed from the animal with myeloma cells. In addition to the above, if necessary, DNAs encoding human antibody-derived light-chain or heavy-chain variable regions and constant regions are produced by a gene recombinant technique or a DNA synthesizer.

In the case of a humanized antibody, DNA in which the CDR coding sequences in a DNA encoding a human antibody-derived light-chain or heavy-chain variable region have been substituted with corresponding CDR coding sequences of an antibody from a non-human animal (e.g., a mouse, a rat, or a chicken) is produced. The DNA obtained as above is ligated to the DNA encoding a constant region of a human antibody-derived light chain or heavy chain. Thus, DNA encoding a humanized antibody can be produced.

In the case of a chimeric antibody, DNA encoding an antibody light-chain or heavy-chain variable region from a non-human animal (e.g., a mouse, a rat, or a chicken) is ligated to the DNA encoding a human antibody-derived light-chain or heavy-chain constant region. Thus, DNA encoding a chimeric antibody can be produced.

A single-chain antibody is an antibody in which a heavy-chain variable region and a light-chain variable region are linearly ligated to each other via a linker. DNA encoding a single-chain antibody can be produced by binding DNA encoding a heavy-chain variable region, DNA encoding a linker, and a DNA encoding a light-chain variable region. Here, a heavy-chain variable region and a light-chain variable region are those from a human antibody or those from a human antibody in which CDRs alone have been substituted with CDRs of an antibody from a non-human animal (e.g., a mouse, a rat, or a chicken). In addition, the linker consists of 12 to 19 amino acids. An example thereof is (G₄S)₃ consisting of 15 amino acids (G. B. Kim et al., Protein Engineering Design and Selection 2007, 20 (9): 425-432).

A bispecific antibody (diabody) is an antibody capable of specifically binding to two different epitopes in which, for example, DNA encoding a heavy-chain variable region A, DNA encoding a light-chain variable region B, DNA encoding a heavy-chain variable region B, and DNA encoding a light-chain variable region A are bound to each other in such order (provided that DNA encoding a light-chain variable region B and DNA encoding a heavy-chain variable region B are bound to each other via DNA encoding a linker described above). Thus, DNA encoding a bispecific antibody can be produced. Here, both a heavy-chain variable region and a light-chain variable region are those from a human antibody or those from a human antibody in which CDRs alone have been substituted with CDRs of an antibody from a non-human animal (e.g., a mouse, a rat, or a chicken).

Recombinant DNA produced as above is incorporated into one or a plurality of appropriate vector(s). Each such vector is introduced into a host cell (e.g., a mammal cell, a yeast cell, or an insect cell) for (co)expression. Thus, a recombinant antibody can be produced (P. J. Delves., ANTIBODY PRODUCTION ESSENTIAL TECHNIQUES., 1997 WILEY, P. Shepherd and C. Dean., Monoclonal Antibodies., 2000 OXFORD UNIVERSITY PRESS; J. W. Goding, Monoclonal Antibodies: Principles and Practice., 1993 ACADEMIC PRESS).

Examples of an antibody of the present invention produced by the above method include the following antibodies (a) to (k).

(a) An antibody comprising a heavy-chain variable region comprising the sequences shown in SEQ ID NOS: 40, 41, and 42 and a light-chain variable region comprising the sequences shown in SEQ ID NOS: 44, 45, and 46 (and preferably an antibody composed of a heavy-chain variable region of SEQ ID NO: 43 and a light-chain variable region of SEQ ID NO: 47).

(b) An antibody comprising a heavy-chain variable region comprising the sequences shown in SEQ ID NOS: 40, 41, and 42 and a light-chain variable region comprising the sequences shown in SEQ ID NOS: 50, 51, and 52 (and preferably an antibody composed of a heavy-chain variable region of SEQ ID NO: 43 and a light-chain variable region of SEQ ID NO: 53).

(c) An antibody comprising a heavy-chain variable region comprising the sequences shown in SEQ ID NOS: 40, 41, and 42 and a light-chain variable region comprising the sequences shown in SEQ ID NOS: 55, 56, and 57 (and preferably an antibody composed of a heavy-chain variable region of SEQ ID NO: 43 and a light-chain variable region of SEQ ID NO: 58).

(d) An antibody comprising a heavy-chain variable region comprising the sequences shown in SEQ ID NOS: 40, 41, and 42 and a light-chain variable region comprising the sequences shown in SEQ ID NOS: 60, 61, and 62 (and preferably an antibody composed of a heavy-chain variable region of SEQ ID NO: 43 and a light-chain variable region of SEQ ID NO: 63).

(e) An antibody comprising a heavy-chain variable region comprising the sequences shown in SEQ ID NOS: 40, 41, and 42 and a light-chain variable region comprising the sequences shown in SEQ ID NOS: 65, 66, and 67 (and preferably an antibody composed of a heavy-chain variable region of SEQ ID NO: 43 and a light-chain variable region of SEQ ID NO: 68).

(f) An antibody comprising a heavy-chain variable region comprising the sequences shown in SEQ ID NOS: 70, 71, and 72 and a light-chain variable region comprising the sequences shown in SEQ ID NOS: 74, 75, and 76 (and preferably an antibody composed of a heavy-chain variable region of SEQ ID NO: 73 and a light-chain variable region of SEQ ID NO: 77).

(g) An antibody comprising a heavy-chain variable region comprising the sequences shown in SEQ ID NOS: 80, 81, and 82 and a light-chain variable region comprising the sequences shown in SEQ ID NOS: 84, 85, and 86 (and preferably an antibody composed of a heavy-chain variable region of SEQ ID NO: 83 and a light-chain variable region of SEQ ID NO: 87).

(h) An antibody comprising a heavy-chain variable region comprising the sequences shown in SEQ ID NOS: 90, 91, and 92 and a light-chain variable region comprising the sequences shown in SEQ ID NOS: 94, 95, and 96 (and preferably an antibody composed of a heavy-chain variable region of SEQ ID NO: 93 and a light-chain variable region of SEQ ID NO: 97).

(i) An antibody comprising a heavy-chain variable region comprising the sequences shown in SEQ ID NOS: 100, 101, and 102 and a light-chain variable region comprising the sequences shown in SEQ ID NOS: 104, 105, and 106 (and preferably an antibody composed of a heavy-chain variable region of SEQ ID NO: 103 and a light-chain variable region of SEQ ID NO: 107).

(j) An antibody comprising a heavy-chain variable region comprising the sequences shown in SEQ ID NOS: 110, 111, and 112 and a light-chain variable region comprising the sequences shown in SEQ ID NOS: 114, 115, and 116 (and preferably an antibody composed of a heavy-chain variable region of SEQ ID NO: 113 and a light-chain variable region of SEQ ID NO: 117).

(k) An antibody comprising a heavy-chain variable region comprising the sequences shown in SEQ ID NOS: 120, 121, and 122 and a light-chain variable region comprising the sequences shown in SEQ ID NOS: 124, 125, and 126 (and preferably an antibody composed of a heavy-chain variable region of SEQ ID NO: 123 and a light-chain variable region of SEQ ID NO: 127).

Here, amino acid sequences shown in SEQ ID NOS: 40, 41, and 42, amino acid sequences shown in SEQ ID NOS: 70, 71, and 72, amino acid sequences shown in SEQ ID NOS: 80, 81, and 82, amino acid sequences shown in SEQ ID NOS: 90, 91, and 92, amino acid sequences shown in SEQ ID NOS: 100, 101, and 102, amino acid sequences shown in SEQ ID NOS: 110, 111, and 112, or amino acid sequences shown in SEQ ID NOS: 120, 121, and 122 correspond to CDR1, CDR2, and CDR3 of mouse antibody heavy-chain variable regions, respectively. In addition, amino acid sequences shown in SEQ ID NOS: 44, 45, and 46, amino acid sequences shown in SEQ ID NOS: 50, 51, and 52, amino acid sequences shown in SEQ ID NOS: 55, 56, and 57, amino acid sequences shown in SEQ ID NOS: 60, 61, and 62, amino acid sequences shown in SEQ ID NOS: 65, 66, and 67, amino acid sequences shown in SEQ ID NOS: 74, 75, and 76, amino acid sequences shown in SEQ ID NOS: 84, 85, and 86, amino acid sequences shown in SEQ ID NOS: 94, 95, and 96, amino acid sequences shown in SEQ ID NOS: 104, 105, and 106, amino acid sequences shown in SEQ ID NOS: 114, 115, and 116, or amino acid sequences shown in SEQ ID NOS: 124, 125, and 126 correspond to CDR1, CDR2, and CDR3 of mouse antibody light-chain variable regions, respectively.

In addition, a humanized antibody, a chimeric antibody, a single-chain antibody, or a bispecific antibody of the present invention is, for example, the following antibody (i) or (ii) (an example of antibody (a) is described below).

(i) An antibody comprising: a heavy-chain variable region comprising the amino acid sequences of SEQ ID NOS: 40, 41, and 42 and an amino acid sequence of a human antibody-derived framework region; and a light-chain variable region comprising the amino acid sequences of SEQ ID NOS: 44, 45, and 46 and amino acid sequences of human antibody-derived framework regions (and preferably an antibody comprising the amino acid sequence of SEQ ID NO: 43 in a heavy-chain variable region and the amino acid sequence of SEQ ID NO: 47 in a light-chain variable region).

(ii) An antibody comprising: a heavy-chain variable region comprising the amino acid sequences of SEQ ID NOS: 40, 41, and 42 and amino acid sequences of human antibody-derived framework regions; a heavy-chain constant region comprising a human antibody-derived amino acid sequence; a light-chain variable region comprising the amino acid sequences of SEQ ID NOS: 44, 45, and 46 and amino acid sequences of human antibody-derived framework regions; and a light-chain constant region comprising a human antibody-derived amino acid sequence (and preferably an antibody comprising: a heavy-chain variable region comprising the amino acid sequence of SEQ ID NO: 43; a heavy-chain constant region comprising a human antibody-derived amino acid sequence; a light-chain variable region comprising the amino acid sequence of SEQ ID NO: 47; and a light-chain constant region comprising a human antibody-derived amino acid sequence).

In addition, sequences of human antibody heavy-chain and light-chain constant and variable regions can be obtained from, for example, NCBI (U.S.A: GenBank, UniGene, etc.). For example, the following sequences can be used as reference sequences for the corresponding regions: the sequence with registration no. J00228 for a human IgG1 heavy-chain constant region; the sequence with registration no. J00230 for a human IgG2 heavy-chain constant region; the sequence with registration no. X03604 for a human IgG3 heavy-chain constant region; the sequence with registration no. K01316 for a human IgG4 heavy-chain constant region; the sequence with registration no. V00557, X64135, or X64133 for a human light-chain κ constant region; and the sequence with registration no. X64132 or X64134 for a human light-chain λ constant region.

The above antibodies preferably have cytotoxic activity, thereby exhibiting antitumor effects.

In addition, the above specific sequences of heavy-chain and light-chain variable regions and CDRs in an antibody are merely described for exemplification. It is obvious that the present invention is not limited to particular sequences. A hybridoma capable of producing a different human antibody or a non-human animal antibody (e.g., a mouse antibody) against human CAPRIN-1 is produced. A monoclonal antibody produced by the hybridoma is collected. Then, it is determined whether or not the obtained antibody is an antibody of interest using, as indicators, immunological binding activity and cytotoxic activity with respect to human CAPRIN-1. Thus, a monoclonal antibody-producing hybridoma of interest is identified. Thereafter, as described above, DNAs encoding heavy-chain and light-chain variable regions of an antibody of interest are produced from the hybridoma for sequence determination. The DNAs are used for production of different antibodies.

Further, the above antibody of the present invention may be any one of antibodies (i) to (iv) above having a substitution, deletion, or addition of one or several (and preferably, 1 or 2) amino acid(s), particularly in a framework region sequence and/or a constant region sequence, as long as it has the specific property of specifically recognizing CAPRIN-1. Here, the term “several amino acids” indicates 2 to 5 and preferably 2 or 3 amino acids.

Furthermore, according to the present invention, DNA encoding the above antibody of the present invention, DNA encoding a heavy chain or light chain of the antibody, or DNA encoding a heavy-chain or light-chain variable region of the antibody is also provided. For instance, in the case of antibody (a), examples of such DNA include: DNA encoding a heavy-chain variable region comprising nucleotide sequences encoding the amino acid sequences of SEQ ID NOS: 40, 41, and, 42; and DNA encoding a light-chain variable region comprising nucleotide sequences encoding the amino acid sequences of SEQ ID NOS: 44, 45, and 46.

Complementarity determining regions (CDRs) encoded by DNAs of the above sequences are regions that determine antibody specificity. Therefore, sequences encoding the other regions (i.e., constant regions and framework regions) in an antibody may be sequences from a different antibody. Here, different antibodies include antibodies from non-human organisms. However, in view of reduction of side effects, human-derived antibodies are preferable. That is to say, in the above case, DNA regions encoding framework regions and constant regions of heavy and light chains preferably comprise nucleotide sequences encoding the relevant amino acid sequences from a human antibody.

Further, different examples of DNA encoding an antibody of the present invention, such as antibody (a), include DNA encoding a heavy-chain variable region comprising a nucleotide sequence encoding the amino acid sequence of SEQ ID NO: 43 and DNA in which a region encoding a light-chain variable region comprises a nucleotide sequence encoding the amino acid sequence of SEQ ID NO: 47. Here, an example of a nucleotide sequence encoding the amino acid sequence of SEQ ID NO: 43 is the nucleotide sequence of SEQ ID NO: 48. In addition, an example of a nucleotide sequence encoding the amino acid sequence of SEQ ID NO: 47 is the nucleotide sequence of SEQ ID NO: 49. Also, the above DNAs encoding heavy-chain and light-chain constant regions preferably comprise nucleotide sequences encoding the corresponding human antibody-derived amino acid sequences.

DNA of the present invention can be obtained by, for example, the aforementioned methods or the following methods. First, total RNA is prepared from a hybridoma for an antibody of the present invention using a commercially available RNA extraction kit. Then, cDNA is synthesized with a reverse transcriptase using random primers and the like. Next, cDNA encoding an antibody is amplified by a PCR method using, as primers, oligonucleotides having sequences conserved in variable regions of known mouse antibody heavy-chain and light-chain genes. Sequences encoding constant regions can be obtained by amplifying known sequences by a PCR method. The nucleotide sequence of the DNA can be determined by a general method involving, for example, incorporation into a plasmid or phage for sequence determination.

It is thought that antitumor effects of an anti-CAPRIN-1 antibody used in the present invention upon CAPRIN-1-expressing cancer cells are exhibited through mechanisms of cytotoxicities described below.

The cytotoxicities are effector cell-mediated antibody-dependent cellular cytotoxicity (ADCC) against CAPRIN-1-expressing cells and complement-dependent cytotoxicity (CDC) against CAPRIN-1-expressing cells.

Accordingly, the activity of an anti-CAPRIN-1 antibody used in the present invention can be evaluated via ex vivo determination of ADCC activity or CDC activity to CAPRIN-1-expressing cancer cells as specifically described in the Examples mentioned below.

An anti-CAPRIN-1 antibody used in the present invention binds to a CAPRIN-1-protein on a cancer cell and exhibits antitumor effects based on the above activity. Therefore, such antibody is believed to be useful for cancer treatment or prevention. Specifically, according to the present invention, the pharmaceutical composition for treatment and/or prevention of cancer that comprises, as an active ingredient, an anti-CAPRIN-1 antibody, is provided. When an anti-CAPRIN-1 antibody is used for the purpose of administering an antibody to humans (antibody treatment), it is preferably used in the form of a human antibody or a humanized antibody in order to reduce immunogenicity.

In addition, as the binding affinity between an anti-CAPRIN-1 antibody and a CAPRIN-1 protein on a cancer cell surface becomes higher, stronger antitumor activity can be exhibited by an anti-CAPRIN-1 antibody. Therefore, if an anti-CAPRIN-1 antibody having high binding affinity to a CAPRIN-1 protein can be obtained, even stronger antitumor effects can be expected to be exhibited. Accordingly, it becomes possible to use such antibody as a pharmaceutical composition for cancer treatment and/or prevention. As described above, for high binding affinity, the affinity constant Ka (kₒₙ/k_{off}) is preferably at least 10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, or at least 10¹³ M⁻¹.

### <Binding to Antigen Expression Cells>

The capacity of an antibody to bind to CAPRIN-1 can be specified via binding assay using, for example, ELISA, a Western blot method, immunofluorescence, or flowcytometry analysis as described in the Examples.

### <Immunohistochemical Staining>

An antibody that recognizes CAPRIN-1 can be tested in terms of reactivity with CAPRIN-1 by an immunohistochemical method known to persons skilled in the art using a frozen tissue section fixed with paraformaldehyde or acetone or a paraffin-embedded tissue section fixed with paraformaldehyde. Such section is prepared from a tissue obtained from a patient during surgery or an animal carrying xenograft tissue that has been inoculated with a natural cell or transfected cell line that expresses CAPRIN-1.

For immunohistochemical staining, an antibody reactive to CAPRIN-1 can be stained by a variety of methods. For example, it can be visualized by reacting with a horseradish peroxidase-conjugated goat anti-mouse antibody or goat anti-rabbit antibody.

### <Pharmaceutical Composition>

A target of the pharmaceutical composition for treatment and/or prevention of cancer of the present invention is not particularly limited as long as the target is a cancer (cell) expressing the CAPRIN-1 gene.

Both the terms “tumor” and “cancer” used herein refer to malignant neoplasm, and thus they are used in an exchangeable manner.

A cancer that can be a target in the present invention is a cancer expressing a gene encoding a polypeptide comprising an amino acid sequence of any one of the even-numbered SEQ ID NOS: 2 to 30 or a partial sequence consisting of 7 or more consecutive amino acids of said amino acid sequence. Preferable examples thereof include breast cancer, brain tumor, leukemia, lung cancer, lymphoma, mastocytoma, esophageal cancer, and colon cancer.

Examples of these specific cancers include, but are not limited to, breast adenocarcinoma, composite type breast adenocarcinoma, malignant mammary mixed tumor, intraductal papillary adenocarcinoma, lung adenocarcinoma, squamous cell cancer, small cell cancer, large cell cancer, glioma that is a tumor of neuroepithelial tissue, ependymoma, neuronal tumor, embryonal neuroectodermal tumor, schwannoma, neurofibroma, meningioma, chronic lymphocytic leukemia, lymphoma, gastrointestinal lymphoma, digestive lymphoma, small-cell-to-medium-cell lymphoma, cecal cancer, ascending colon cancer, descending colon cancer, transverse colon cancer, sigmoid colon cancer, and rectal cancer.

In addition, the subject animal of the present invention is a mammal. Examples thereof include mammals such as primates, pet animals, livestock animals, and sport animals. Humans, dogs, and cats are particularly preferable.

When an antibody used in the present invention is used as a pharmaceutical composition, it can be formulated by a method known to persons skilled in the art. For instance, it can be parenterally used in the form of a parenteral injection of: an aseptic solution comprising water or a pharmacologically acceptable non-water solution; or a suspension liquid. For example, in one possible case, it can be formulated with the combined use of a pharmacologically acceptable carrier or medium and specifically sterilized water, physiological saline, plant oil, an emulsifier, a suspension, a surfactant, a stabilizer, a flavoring agent, an excipient, a vehicle, a preservative, or a binder in an appropriate manner by mixing in a unit dosage form required for a generally acceptable pharmaceutical formulation. The amount of an active ingredient in a formulation is determined such that an appropriate dosage within the indicated range can be achieved.

An aseptic composition for injection purposes can be formulated in accordance with general formulation practice using a vehicle such as distilled water for injection purposes.

Examples of an aqueous solution for injection purposes include physiological saline and isotonic solutions comprising glucose and other adjuvants such as D-sorbitol, D-mannose, D-mannitol, and sodium chloride. Such solution may be used with an appropriate dissolution aid. Examples of such dissolution aid include alcohols such as ethanol and polyalcohol, propylene glycol, polyethylene glycol, and nonion surfactants such as polysorbate 80™ and HCO-60.

Examples of oily liquid include sesame oil and soybean oil. Such oily liquid may be used in combination with a dissolution aid such as benzyl benzoate or benzyl alcohol. In addition, it may be mixed with a buffering agent such as a phosphate buffer solution, a sodium acetate buffer solution, a soothing agent such as procaine hydrochloride, a stabilizer such as benzyl alcohol, phenol, or an antioxidant. In general, a formulated injection solution is introduced into an adequate ample.

The above pharmaceutical composition is orally or parenterally administered. Preferably, it is parenterally administered. Specific examples of dosage forms include injectable agents, intranasally-administered agents, transpulmonarily-administered agents, and percutaneously-administered agents. For example, injectable agents can be systemically or locally administered via intravenous injection, intramuscular injection, intraperitoneal injection, or subcutaneous injection.

In addition, the administration method can be appropriately determined depending on patient age, weight, gender, and symptoms. A single dose of a pharmaceutical composition comprising an antibody or a polynucleotide encoding an antibody can be selected within a range of, for example, 0.0001 mg to 1000 mg per kg of body weight. Alternatively, the dose can be selected within a range of, for example, 0.001 to 100000 mg per patient's body; however, it is not necessarily limited thereto. The dose and the administration method are changed depending on patient age, weight, gender, and symptoms. However, persons skilled in the art can appropriately select the dose and the method.

### <Polypeptide and DNA>

According to the present invention, the following polypeptides and DNAs for antibodies (a) to (k) described above are further provided.

(i) A polypeptide comprising the amino acid sequences of SEQ ID NO: 43, SEQ ID NO: 73, SEQ ID NO: 83, SEQ ID NO: 93, SEQ ID NO: 103, SEQ ID NO: 113, and SEQ ID NO: 123, and DNA encoding the polypeptide.

(ii) A polypeptide comprising the amino acid sequences of SEQ ID NO: 47, SEQ ID NO: 53, SEQ ID NO: 58, SEQ ID NO: 63, SEQ ID NO: 68, SEQ ID NO: 77, SEQ ID NO: 87, SEQ ID NO: 97, SEQ ID NO: 107, SEQ ID NO: 117, and SEQ ID NO: 127, and DNA encoding the polypeptide.

(iii) DNA comprising the nucleotide sequences of SEQ ID NO: 48, SEQ ID NO: 78, SEQ ID NO: 88, SEQ ID NO: 98, SEQ ID NO: 108, SEQ ID NO: 118, and SEQ ID NO: 128.

(iv) DNA comprising the nucleotide sequences of SEQ ID NO: 49, SEQ ID NO: 54, SEQ ID NO: 59, SEQ ID NO: 64, SEQ ID NO: 69, SEQ ID NO: 79, SEQ ID NO: 89, SEQ ID NO: 99, SEQ ID NO: 109, SEQ ID NO: 119, and SEQ ID NO: 129.

(v) A heavy-chain CDR polypeptide comprising amino acid sequences selected from the group consisting of amino acid sequences of SEQ ID NOS: 40, 41, and 42, amino acid sequences of SEQ ID NOS: 70, 71, and 72, amino acid sequences of SEQ ID NOS: 80, 81, and 82, amino acid sequences of SEQ ID NOS: 90, 91, and 92, amino acid sequences of SEQ ID NOS: 100, 101, and 102, amino acid sequences of SEQ ID NOS: 110, 111, and 112, and amino acid sequences of SEQ ID NOS: 120, 121, and 122, and DNA encoding the polypeptide.

(vi) A light-chain CDR polypeptide comprising amino acid sequences selected from the group consisting of amino acid sequences of SEQ ID NOS: 44, 45, and 46, amino acid sequences of SEQ ID NOS: 50, 51, and 52, amino acid sequences of SEQ ID NOS: 55, 56, and 57, amino acid sequences of SEQ ID NOS: 60, 61, and 62, amino acid sequences of SEQ ID NOS: 65, 66, and 67, amino acid sequences of SEQ ID NOS: 74, 75, and 76, amino acid sequences of SEQ ID NOS: 84, 85, and 86, amino acid sequences of SEQ ID NOS: 94, 95, and 96, amino acid sequences of SEQ ID NOS: 104, 105, and 106, amino acid sequences of SEQ ID NOS: 114, 115, and 116, and amino acid sequences of SEQ ID NOS: 124, 125, and 126, and DNA encoding the polypeptide.

These polypeptides and DNAs can be produced by a gene recombinant technique as described above.

### EXAMPLES

The present invention is hereafter described in greater detail with reference to the following examples, although the scope of the present invention is not limited thereto.

### Example 1: Identification of New Cancer Antigen Protein by SEREX Method

(1) Construction of cDNA Library

Total RNA was extracted from a testis tissue of a healthy dog by an Acid guanidium-Phenol-Chloroform method and then a polyA RNA was purified according to protocols included with an Oligotex-dT30 mRNA purification Kit (Takara Shuzo Co., Ltd.).

A canine testis cDNA phage library was synthesized using the thus obtained mRNA (5 μg). The cDNA phage library was constructed using a cDNA Synthesis Kit, a ZAP-cDNA Synthesis Kit, and a ZAP-cDNA GigapackIII Gold Cloning Kit (STRATAGENE) according to protocols included with the kits. The size of the thus constructed cDNA phage library was 7.73×10⁵ pfu/ml.

(2) Screening of cDNA Library Using Serum

Immunoscreening was performed using the above constructed canine testis cDNA phage library. Specifically, host Escherichia coli (XL1-Blue MRF′) was infected with the phage on an NZY agarose plate (Φ90×15 mm) so as to obtain 2210 clones. E. coli cells were cultured at 42° C. for 3 to 4 hours to form plaques. The plate was covered with a nitrocellulose membrane (Hybond C Extra: GE Healthcare Bio-Science) impregnated with IPTG (isopropyl-β-D-thiogalactoside) at 37° C. for 4 hours, so that the protein was induced, expressed, and then transferred to the membrane. Subsequently, the membrane was collected and then immersed in TBS (10 mM Tris-HCl, 150 mM NaCl, and pH 7.5) containing 0.5% powdered skim milk, followed by overnight shaking at 4° C., thereby suppressing nonspecific reaction. The filter was reacted with a 500-fold diluted serum of a canine patient at room temperature for 2 to 3 hours.

As the above serum of a canine patient, a serum collected from a canine patient with breast cancer was used. These sera were stored at −80° C. and then subjected to pre-treatment immediately before use. A method for pretreatment of serum is as follows. Specifically, host Escherichia coli (XL1-Blue MRF′) was infected with a λ ZAP Express phage in which no foreign gene had been inserted and then cultured overnight on a NZY plate medium at 37° C. Subsequently, buffer (0.2 M NaHCO₃ and pH 8.3) containing 0.5 M NaCl was added to the plate, the plate was left to stand at 4° C. for 15 hours, and then a supernatant was collected as an Escherichia coli/phage extract. Next, the thus collected Escherichia coli/phage extract was applied to an NHS-column (GE Healthcare Bio-Science), so that an Escherichia coli-phage-derived protein was immobilized. The serum of a canine patient was applied to the protein-immobilized column for reaction and then Escherichia coli and an antibody adsorbed to the phage were removed from the serum. The serum fraction that had passed through the column was diluted 500-fold with TBS containing 0.5% powdered skim milk. The resultant was used as an immunoscreening material.

A membrane onto which the treated serum and the above fusion protein had been blotted was washed 4 times with TBS-T (0.05% Tween20/TBS) and then caused to react with goat anti-dog IgG (Goat anti-Dog IgG-h+I HRP conjugated (BETHYL Laboratories)) diluted 5000-fold with TBS containing 0.5% powdered skim milk as a secondary antibody for 1 hour at room temperature. Detection was performed via an enzyme coloring reaction using an NBT/BCIP reaction solution (Roche). Colonies that matched sites positive for a coloring reaction were collected from the NZY agarose plate (Φ90×15 mm) and then lysed in 500 μl of an SM buffer (100 mM NaCl, 10 mM MgClSO₄, 50 mM Tris-HCl, 0.01% gelatin, and pH 7.5). Until colonies positive for coloring reaction were unified, secondary screening and tertiary screening were repeated so that 30,940 phage clones reacting with serum IgG were screened for by a method similar to the above. Thus, 5 positive clones were isolated.

### (3) Homology Search for Isolated Antigen Gene

For nucleotide sequence analysis of the 5 positive clones isolated by the above method, a procedure for conversion from phage vectors to plasmid vectors was performed. Specifically, 200 μl of a solution was prepared to contain host Escherichia coli (XL1-Blue MRF′) so that absorbance OD₆₀₀ was 1.0. The solution was mixed with 250 μl of a purified phage solution and then with 1 μl of an ExAssist helper phage (STRATAGENE), followed by 15 minutes of reaction at 37° C. Three (3) ml of LB medium was added and then culture was performed at 37° C. for 2.5 to 3 hours. Immediately after culture, the temperature of the solution was kept at 70° C. by water bath for 20 minutes, centrifugation was performed at 4° C. and 1000×g for 15 minutes, and then the supernatant was collected as a phagemid solution. Subsequently, 200 μl of a solution was prepared to contain phagemid host Escherichia coli (SOLR) so that absorbance OD₆₀₀ was 1.0. The solution was mixed with 10 μl of a purified phage solution, followed by 15 minutes of reaction at 37° C. The solution (50 μl) was seeded on LB agar medium containing ampicillin (final concentration of 50 μg/ml) and then cultured overnight at 37° C. Transformed SOLR single colony was collected and then cultured in LB medium containing ampicillin (final concentration: 50 μg/ml) at 37° C. A plasmid DNA containing the insert of interest was purified using a QIAGEN plasmid Miniprep Kit (QIAGEN).

The purified plasmid was subjected to analysis of the full-length insert sequence by a primer walking method using the T3 primer of SEQ ID NO: 31 and the T7 primer of SEQ ID NO: 32. As a result of sequence analysis, the gene sequences of SEQ ID NOS: 5, 7, 9, 11, and 13 were obtained. A homology search program, BLAST search (ncbi.nlm.nih.gov/BLAST/), was performed using the nucleotide sequences of the genes and the corresponding amino acid sequences (SEQ ID NOS: 6, 8, 10, 12, and 14). As a result of this homology search with known genes, it was revealed that all of the 5 obtained genes encoded CAPRIN-1. Regarding regions to be translated to proteins, the sequence identity among the 5 genes was 100% in terms of nucleotide sequence and 99% in terms of amino acid sequence. Also, regarding regions to be translated to proteins, the sequence identity between the genes and genes encoding human factors homologous thereto (human homologs) was 94% in terms of nucleotide sequence and 98% in terms of amino acid sequence. The nucleotide sequences of the human homologues are shown in SEQ ID NOS: 1 and 3 and the amino acid sequences of the same are shown in SEQ ID NOS: 2 and 4. Also, regarding regions to be translated to proteins, the sequence identity between the thus obtained canine genes and a gene encoding a cattle homologue was 94% in terms of nucleotide sequence and 97% in terms of amino acid sequence. The nucleotide sequence of the cattle homologue is shown in SEQ ID NO: 15 and the amino acid sequence of the same is shown in SEQ ID NO: 16. In addition, regarding regions to be translated to proteins, the sequence identity between the genes encoding the human homologues and the gene encoding the cattle homologue was 94% in terms of nucleotide sequence and ranged from 93% to 97% in terms of amino acid sequence. Also, regarding regions to be translated to proteins, the sequence identity between the obtained canine genes and a gene encoding an equine homologue was 93% in terms of nucleotide sequence and 97% in terms of amino acid sequence. The nucleotide sequence of the equine homologue is shown in SEQ ID NO: 17 and the amino acid sequence of the same is shown in SEQ ID NO: 18. In addition, regarding regions to be translated to proteins, the sequence identity between the genes encoding the human homologues and the gene encoding the equine homologue was 93% in terms of nucleotide sequence and 96% in terms of amino acid sequence. Also, regarding regions to be translated to proteins, the sequence identity between the obtained canine genes and genes encoding mouse homologues ranged from 87% to 89% in terms of nucleotide sequence and ranged from 95% to 97% in terms of amino acid sequence. The nucleotide sequences of the mouse homologues are shown in SEQ ID NOS: 19, 21, 23, 25, and 27 and the amino acid sequences of the same are shown in SEQ ID NOS: 20, 22, 24, 26, and 28. In addition, regarding regions to be translated to proteins, the sequence identity between the genes encoding the human homologues and the genes encoding the mouse homologues ranged from 89% to 91% in terms of nucleotide sequence and ranged from 95% to 96% in terms of amino acid sequence. Also, regarding regions to be translated to proteins, the sequence identity between the obtained canine genes and a gene encoding a chicken homologue was 82% in terms of nucleotide sequence and 87% in terms of amino acid sequence. The nucleotide sequence of the chicken homologue is shown in SEQ ID NO: 29 and the amino acid sequence of the same is shown in SEQ ID NO: 30. In addition, regarding regions to be translated to proteins, the sequence identity between the genes encoding the human homologues and the gene encoding the chicken homologue ranged from 81% to 82% in terms of nucleotide sequence and was 86% in terms of amino acid sequence.

### (4) Gene Expression Analysis in Each Tissue

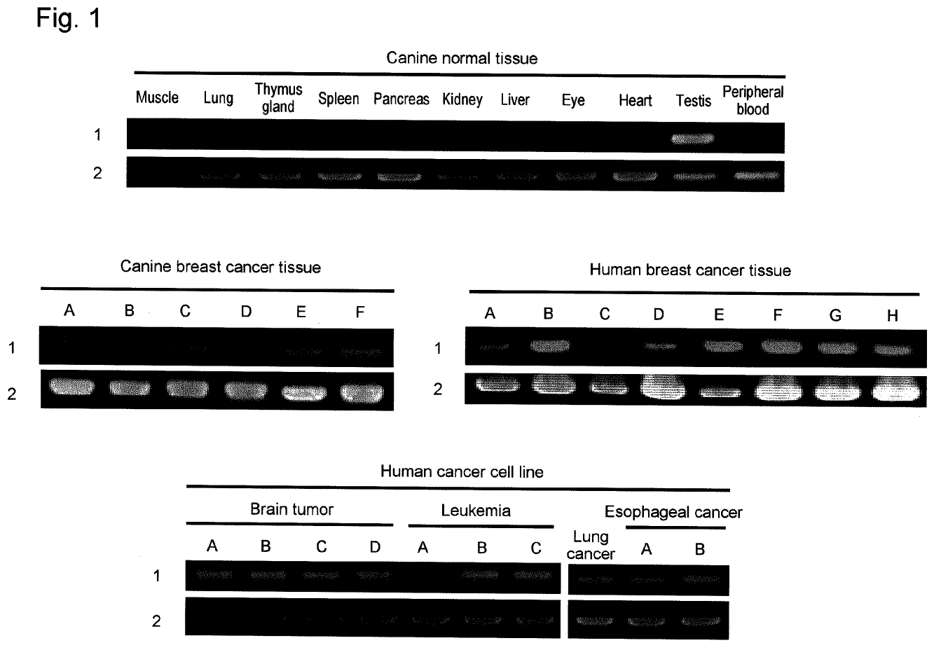

Expression of the genes obtained by the above method in canine and human normal tissues and various cell lines was examined by an RT-PCR method. A reverse transcription reaction was performed as follows. Specifically, total RNA was extracted from each tissue (50 mg to 100 mg) and each cell line (5 to 10×10⁶ cells) using a TRIZOL reagent (Invitrogen) according to protocols included therewith. cDNA was synthesized using the total RNA and Superscript First-Strand Synthesis System for RT-PCR (Invitrogen) according to protocols included therewith. PCR was performed as follows using primers specific to the obtained genes (SEQ ID NOS: 33 and 34). Specifically, PCR was performed by repeating 30 times a cycle of 94° C./30 seconds, 60° C./30 seconds, and 72° C./30 seconds using a Thermal Cycler (BIO RAD) and a reaction solution adjusted to a total amount of 25 μl through addition of each reagent and an attached buffer (0.25 μl of a sample prepared by reverse transcription reaction, the above primers (2 μM each), dNTP (0.2 mM each), and 0.65 U of ExTaq polymerase (Takara Shuzo)). In addition, the gene-specific primers mentioned above were used to amplify the region between nucleotide number 206 and nucleotide number 632 in the nucleotide sequence (canine CAPRIN-1 gene) of SEQ ID NO: 5 and the region between nucleotide number 698 and nucleotide number 1124 in the nucleotide sequence (human CAPRIN-1 gene) of SEQ ID NO: 1. For comparison control, GAPDH-specific primers (SEQ ID NOS: 35 and 36) were used at the same time. As a result, as shown in FIG. 1, strong expression was observed in testis in the case of healthy canine tissues, while expression was observed in canine breast cancer and adenocarcinoma tissues. Furthermore, expression of the human homologs homologous to the obtained genes was also confirmed. As a result, similarly to the case of canine CAPRIN-1 genes, expression could be confirmed only in the testis in the case of normal tissues. However, in the case of cancer cells, expression was detected in many types of cancer cell lines, such as cell lines of breast cancer, brain tumor, leukemia, lung cancer, and esophageal cancer. Expression was confirmed in a particularly large number of breast cancer cell lines. Based on the results, it was confirmed that CAPRIN-1 expression was not observed in normal tissues other than those of the testis while CAPRIN-1 was expressed in many cancer cells and particularly in breast cancer cell lines.

In addition, in FIG. 1, Reference No. 1 along the longitudinal axis indicates the expression pattern of each of the above-identified genes and Reference No. 2 along the same indicates the expression pattern of the GAPDH gene for comparison control.

### (5) Preparation of Polyclonal Antibody Against CAPRIN-1-Derived Peptide

To obtain an antibody binding to CAPRIN-1, CAPRIN-1-derived peptide (Arg-Asn-Leu-Glu-Lys-Lys-Lys-Gly-Lys-Leu-Asp-Asp-Tyr-Gln (SEQ ID NO: 37)) was synthesized. The peptide (1 mg) as an antigen was mixed with an incomplete Freund's adjuvant (IFA) solution in an amount equivalent to the peptide. The mixture was subcutaneously administered to a rabbit 4 times every 2 weeks. Subsequently, blood was collected, so that an antiserum containing a polyclonal antibody was obtained. Furthermore, the antiserum was purified using a protein G support (GE Healthcare Bio-Sciences) and then a polyclonal antibody against the CAPRIN-1-derived peptide was obtained. In addition, an antibody obtained by purifying serum of rabbits to which no antigen had been administered with the use of a protein G support in the manner described above was designated as a control antibody.

### (6) Analysis of Antigen Protein Expression on Cancer Cells

Next, it was examined whether or not the CAPRIN-1 protein was expressed on cell surfaces of 7 types of breast cancer cell lines (MDA-MB-157, T47D, MRK-nu-1, MDA-MB-231V, BT20, SK-BR-3, and MDA-MB-231T) in which CAPRIN-1 gene expression had been strongly confirmed. Each human breast cancer cell line in which gene expression had been confirmed (10⁶ cells) as described above was centrifuged in a 1.5-ml microcentrifugal tube. The polyclonal antibody against the CAPRIN-1-derived peptide (2 μg)(5 μl) prepared in (5) above was added thereto. The resultant was further suspended in PBS containing 0.1% fetal bovine serum (95 μl) and then left to stand on ice for 1 hour. After washing with PBS, the resultant was suspended in PBS containing an FITC-labeled goat anti-rabbit IgG antibody (Santa Cruz Biotechnology, Inc.)(5 μl) and 0.1% fetal bovine serum (FBS) (95 μl) and then left to stand on ice for 1 hour. After washing with PBS, fluorescence intensity was measured using FACSCalibur (Becton, Dickinson and Company). Meanwhile, a procedure similar to the above was performed using the control antibody prepared in (5) above instead of the polyclonal antibody against a CAPRIN-1-derived peptide, so that a control was prepared. As a result, fluorescence intensity was found to be at least 30% stronger in all cells to which the anti-human CAPRIN-1 antibody had been added than that in control cells. Specifically, the following increases in fluorescence intensity were confirmed: MDA-MB-157: 184%; T47D: 221%; MRK-nu-1: 115%; MDA-MB-231V: 82%; BT20: 32%; SK-BR-3: 279%; and MDA-MB-231T: 80%. Based on the above, it was confirmed that the CAPRIN-1 protein was expressed on the cell surfaces of the above human cancer cell lines. In addition, the rate of increase in fluorescence intensity is represented by the rate of increase in mean fluorescence intensity (MFI value) in cells. It was calculated by the following equation.

$$Rate of increase in mean fluorescence intensity ( rate of increase in fluorescence intensity ) ( \% ) = ( ( MFI value of cells reacted with anti - human CAPRIN - 1 antibody ) - ( control MFI value ) ) / ( control MFI value ) \times 100$$

### (7) Immunohistochemical Staining

### (7)-1 CAPRIN-1 Expression in Normal Mouse and Canine Tissues

Mice (Balb/c, female) and dogs (beagle dogs, female) were exsanguinated under ether anesthesia and ketamine/isoflurane anesthesia. After laparotomy, organs (stomach, liver, eyeball, thymus gland, muscle, bone marrow, uterus, small intestine, esophagus, heart, kidney, salivary gland, large intestine (colon), mammary gland, brain, lung, skin, adrenal gland, ovary, pancreas, spleen, and bladder) were each transferred to a 10-cm dish containing PBS. Each organ was cut open in PBS and then subjected to perfusion fixation overnight with 0.1 M phosphate buffer (pH 7.4) containing 4% paraformaldehyde (PFA). The perfusate was discarded, the tissue surface of each organ was rinsed with PBS, and then a PBS solution containing 10% sucrose was added to a 50-ml centrifugal tube. Each tissue was then placed in each tube and then shaken using a rotor at 4° C. for 2 hours. Each solution was substituted with a PBS solution containing 20% sucrose and then left to stand at 4° C. until tissues precipitated. Each solution was substituted with a PBS solution containing 30% sucrose and then left to stand at 4° C. until tissues precipitated. Each tissue was removed and a necessary portion was excised with a surgical scalpel. Next, an OCT compound (Tissue Tek) was applied and spread over each tissue surface, and then the tissues were placed on Cryomold. Cryomold was placed on dry ice for rapid freezing. Tissues were sliced into sections of 10 to 20 μm thickness using a cryostat (LEICA) and then the sliced tissue sections were air-dried on glass slides for 30 minutes using a hair dryer, so that glass slides on which sliced tissue sections had been placed were prepared. Next, each glass slide was placed in a staining bottle filled with PBS-T (saline containing 0.05% Tween20), so that a procedure involving exchange with PBS-T every 5 minutes was performed 3 times. Excess water around each specimen was removed using Kimwipes and then each section was encircled using DAKOPEN (DAKO). As blocking solutions, a MOM mouse Ig blocking reagent (VECTASTAIN) was applied onto mouse tissue and a PBS-T solution containing 10% FBS was applied onto canine tissue. The resultants were left to stand in a moist chamber at room temperature for 1 hour. Next, a solution was prepared to contain a monoclonal antibody (monoclonal antibody #6) against CAPRIN-1 having the heavy-chain variable region of SEQ ID NO: 73 and the light-chain variable region of SEQ ID NO: 77 and reacting with the cancer cell surfaces prepared in Example 4, which antibody was adjusted at a concentration of 10 μg/ml in the blocking solution. The solution was applied onto each slide glass and then left to stand within a moist chamber at 4° C. overnight. After 3 times wash, each 10 minutes, with PBS-T, a MOM biotin-labeled anti-IgG antibody (VECTASTAIN) diluted 250-fold with the blocking solution was applied onto each glass slide and then left to stand within a moist chamber at room temperature for 1 hour. After 3 times wash, each 10 minutes, with PBS-T, an avidin-biotin ABC reagent (VECTASTAIN) was applied and then left to stand within a moist chamber at room temperature for 5 minutes. After 3 times wash, each 10 minutes, with PBS-T, a DAB staining solution (DAB 10 mg+30% H₂O₂ 10 μl/0.05 M Tris-HCl (pH 7.6) 50 ml) was applied and then the glass slides were left to stand within a moist chamber at room temperature for 30 minutes. Glass slides were rinsed with distilled water and then a hematoxylin reagent (DAKO) was applied. After being left to stand at room temperature for 1 minute, the glass slides were rinsed with distilled water. The glass slides were immersed in 70%, 80%, 90%, 95%, and 100% ethanol solutions in such order for 1 minute each and then left to stand in xylene overnight. The glass slides were removed, coverslipped with Glycergel Mounting Medium (DAKO), and then observed. As a result, CAPRIN-1 expression was observed to a slight degree within cells in all salivary gland, kidney, colon, and stomach tissues, but CAPRIN-1 expression was never observed on cell surfaces. Also, absolutely no CAPRIN-1 expression was observed in tissues from other organs. In addition, similar results were obtained when the monoclonal antibody against CAPRIN-1 having the heavy-chain variable region of SEQ ID NO: 103 and the light-chain variable region of SEQ ID NO: 107 (monoclonal antibody #9) was used.

### (7)-2 CAPRIN-1 Expression in Canine Breast Cancer Tissue

With the use of 108 frozen canine breast cancer tissue specimens from dogs diagnosed by pathological diagnosis as having malignant breast cancer, frozen section slides were prepared by a method similar to the above and immunohistochemical staining was performed using the monoclonal antibody #6 prepared in Example 4. As a result, CAPRIN-1 expression was confirmed in 100 out of the 108 specimens (92.5%). CAPRIN-1 was particularly strongly expressed on the surfaces of highly atypical cancer cells. In addition, similar results were obtained when the monoclonal antibody #9 produced in Example 4 was used.

### (7)-3 CAPRIN-1 Expression in Human Breast Cancer Tissue

Immunohistochemical staining was performed using 188 breast cancer tissue specimens of a paraffin-embedded human breast cancer tissue array (BIOMAX). After 3 hours of treatment at 60° C., the human breast cancer tissue array was added to a staining bottle filled with xylene and then xylene replacement every 5 minutes was performed 3 times. Next, a similar procedure was performed using ethanol and PBS-T instead of xylene. The human breast cancer tissue array was added to a staining bottle filled with 10 mM citrate buffer (pH 6.0) containing 0.05% Tween20, treated for 5 minutes at 125° C., and then left to stand at room temperature for 40 minutes or more. Excess water around each specimen was removed using Kimwipes, each section was encircled using DAKOPEN, and then an appropriate amount of Peroxidase Block (DAKO) was added dropwise. The resultant was left to stand at room temperature for 5 minutes and then added to a staining bottle filled with PBS-T. PBS-T replacement every 5 minutes was performed 3 times. As a blocking solution, a PBS-T solution containing 10% FBS was applied and then left to stand within a moist chamber at room temperature for 1 hour. Next, a solution was prepared to contain the monoclonal antibody #6 reacting with the cancer cell surfaces prepared in Example 4 at a concentration of 10 μg/ml adjusted using a PBS-T solution containing 5% FBS. The solution was applied and then left to stand overnight within a moist chamber at 4° C. After 3 times wash, each 10 minutes, with PBS-T, an appropriate amount of Peroxidase Labeled Polymer Conjugated (DAKO) was added dropwise, and then the glass slides were left to stand at room temperature for 30 minutes within a moist chamber. After 3 times wash, each 10 minutes, with PBS-T, a DAB staining solution (DAKO) was applied and then left to stand at room temperature for 10 minutes. The DAB staining solution was discarded and then 10 minutes of wash was performed with PBS-T for 3 times. The glass slides were rinsed with distilled water and then immersed in 70%, 80%, 90%, 95%, and 100% ethanol solutions in such order for 1 minute each and then left to stand in xylene overnight. The glass slides were removed, coverslipped with Glycergel Mounting Medium (DAKO), and then observed. As a result, strong CAPRIN-1 expression was observed for 138 (73%) out of the total 188 breast cancer tissue specimens. In addition, similar results were obtained when the monoclonal antibody #9 prepared in Example 4 was used.

### (7)-4 CAPRIN-1 Expression in Human Malignant Brain Tumor

With the use of 247 malignant brain tumor tissue specimens of paraffin-embedded human malignant brain tumor tissue arrays (BIOMAX), immunohistochemical staining was performed by a method similar to that in (7)-3 above using the monoclonal antibody #6 prepared in Example 4. As a result, strong CAPRIN-1 expression was observed in 227 (92%) out of the total 247 malignant brain tumor tissue specimens. In addition, similar results were obtained when the monoclonal antibody #9 prepared in Example 4 was used.

### (7)-5 CAPRIN-1 Expression in Human Breast Cancer Metastatic Lymph Node

With the use of 150 tissue specimens of human breast cancer metastatic lymph nodes of paraffin-embedded human breast cancer metastatic lymph node tissue arrays (BIOMAX), immunohistochemical staining was performed by a method similar to that in (7)-3 above using the monoclonal antibody #6 prepared in Example 4. As a result, strong CAPRIN-1 expression was observed in 136 (90%) out of the total 150 tissue specimens of human breast cancer metastatic lymph nodes. Specifically, it was revealed that CAPRIN-1 is also strongly expressed in a cancer tissue that has metastasized from breast cancer. In addition, similar results were obtained when the monoclonal antibody #9 prepared in Example 4 was used.

### Example 2: Antitumor Effects (ADCC Activity) of Antibody Against CAPRIN-1 Upon Cancer Cells

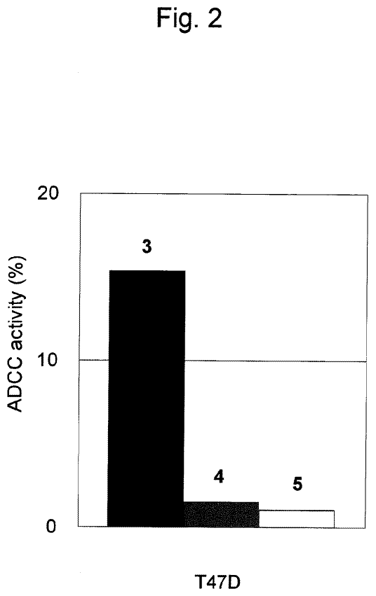

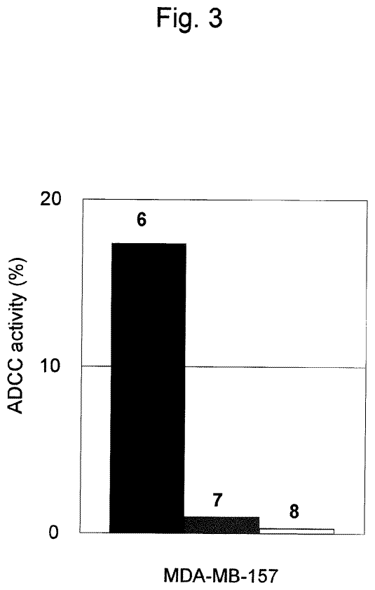

Next, it was examined whether or not an antibody against CAPRIN-1 would be able to damage CAPRIN-1-expressing tumor cells. Evaluation was carried out using the polyclonal antibody against a human CAPRIN-1-derived peptide prepared in Example 1. Two types of human breast cancer cell lines (T47D and MDA-MB-157) (10⁶ cells each), in which CAPRIN-1 expression had been confirmed, were separately collected into a 50-ml centrifugal tube. Chromium 51 (100 μCi) was added thereto, followed by incubation at 37° C. for 2 hours. Thereafter, cells were washed 3 times with an RPMI1640 medium containing 10% fetal calf serum and added to wells (10³ cells per well) in 96-well V-bottom plates. The above polyclonal antibody against a human CAPRIN-1-derived peptide was added thereto (1 μg per well). Further, lymphocytes separated from rabbit peripheral blood were added thereto (2×10⁵ cells per well), followed by culture under conditions of 37° C. and 5% CO₂ for 4 hours. After culture, the level of chromium (Cr) 51 released from damaged tumor cells in each culture supernatant was determined. Then, the ADCC activity of the polyclonal antibody against a human CAPRIN-1-derived peptide to cancer cells was calculated. As a result, ADCC activities against T47D (15.4%) and MDA-MB-157 (17.3%) were confirmed (see FIGS. 2 and 3). Meanwhile, substantially no activity was observed in a case in which a procedure similar to the above was performed using the control antibody prepared from peripheral blood of a rabbit that had not been immunized with an antigen (Example 1 (5)) or in a case in which no antibody was added (see FIGS. 2 and 3). Accordingly, it was revealed that CAPRIN-1-expressing tumor cells can be damaged by inducing ADCC activity with the use of an antibody against CAPRIN-1.

In addition, for cytotoxic activity, an antibody against CAPRIN-1 used in the present invention, mouse lymphocytes, and 10³ cells incorporating chromium 51 from a leukemia cell line were mixed together and cultured for 4 hours. Thereafter, the level of chromium 51 released into the medium was determined. Then, the cytotoxic activity to the leukemia cell line was calculated by the following equation*.

$${}^{*} Equation$$

$$Cytotoxic activity ( \% ) = [ \text{} ( the level of chromium 51 released from T 47 D or MDA - MB - 157 to which an antibody against CAPRIN - 1 and mouse lymphocytes were added ) / ( the level of chromium 51 released from target cells to which 1 N hydrochloric acid was added ) ] \times 100$$

### Example 3: Preparation of New Human Cancer Antigen Proteins

### (1) Preparation of Recombinant Protein

A recombinant protein of a human homolog gene was prepared by the following method based on the gene of SEQ ID NO: 1 obtained in Example 1. PCR was performed by repeating 30 times a cycle of 98° C./10 seconds and 68° C./2.5 minutes using a Thermal Cycler (BIO RAD) and a reaction solution adjusted to a total amount of 50 μl through addition of each reagent and an attached buffer (1 μl of cDNA (which was from a variety of tissue/cell-derived cDNAs prepared in Example 1 and observed for their expression by RT-PCR), 2 types of primers (0.4 μM each; SEQ ID NOS: 38 and 39) containing SacI and XhoI restriction enzyme cleavage sequences, 0.2 mM dNTP, 1.25 U PrimeSTAR HS polymerase (Takara Shuzo)). The above 2 types of primers were used to amplify the region encoding the full-length amino acid sequence of SEQ ID NO: 2. After PCR, the thus amplified DNA was subjected to 1% agarose gel electrophoresis and then a DNA fragment of about 2.1 kbp was purified using a QIAquick Gel Extraction Kit (QIAGEN).

The purified DNA fragment was ligated to a pCR-Blunt cloning vector (Invitrogen). The vector was transformed into Escherichia coli and then the plasmid was collected. It was confirmed based on the sequence that the amplified gene fragment matched the target sequence. The plasmid that matched the sequence of interest was treated with SacI and XhoI restriction enzymes and then the resultant was purified using a QIAquick Gel Extraction Kit. Then, the gene sequence of interest was inserted into a pET30a expression vector (Novagen) for Escherichia coli treated with SacI and XhoI restriction enzymes. A His tag-fused recombinant protein can be produced using the vector. The plasmid was transformed into Escherichia coli BL21 (DE3) for expression and then expression induction was performed using 1 mM IPTG, so that the target protein was expressed within Escherichia coli.4

### (2) Purification of Recombinant Protein

Each above-obtained recombinant Escherichia coli expressing SEQ ID NO: 1 was cultured at 37° C. in LB medium containing 30 μg/ml kanamycin until the absorbance at 600 nm reached around 0.7. Then isopropyl-β-D-1-thiogalactopyranoside was added to a final concentration of 1 mM, followed by 4 hours of culture at 37° C. Subsequently, cells were collected by 10 minutes of centrifugation at 4800 rpm. The cell pellet was suspended in phosphate buffered saline and then centrifuged at 4800 rpm for 10 minutes for washing cells.

The cells were suspended in phosphate buffered saline and then subjected to ultrasonication on ice. The thus ultrasonicated Escherichia coli lysate was centrifuged at 6000 rpm for 20 minutes. The thus obtained supernatant was used as a soluble fraction and the thus obtained precipitate was used as an insoluble fraction.

The soluble fraction was added to a nickel chelate column (carrier: Chelating Sepharose (TradeMark) Fast Flow (GE Healthcare), column capacity: 5 mL, 50 mM hydrochloric acid buffer (pH 8.0) as equilibrated buffer)) prepared according to a conventional method. The unadsorbed fraction was washed with 50 mM hydrochloric acid buffer (pH 8.0) in an amount 10 times the capacity of the column and 20 mM phosphate buffer (pH 8.0) containing 20 mM imidazole. Immediately after washing, 6 beds were eluted with 20 mM phosphate buffer (pH 8.0) containing 100 mM imidazole. An elution fraction of 20 mM phosphate buffer (pH 8.0) containing 100 mM imidazole (for which the elution of the protein of interest had been confirmed by Coomassie staining) was added to a strong anion exchange column (carrier: Q Sepharose (TradeMark) Fast Flow (GE Healthcare), column capacity: 5 mL, and 20 mM phosphate buffer (pH 8.0) as equilibrated buffer). The unadsorbed fraction was washed with 20 mM phosphate buffer (pH 7.0) in an amount 10 times the column capacity and 20 mM phosphate buffer (pH 7.0) containing 200 mM sodium chloride. Immediately after washing, 5 beds were eluted using 20 mM phosphate buffer (pH 7.0) containing 400 mM sodium chloride. Thus, purified fractions of proteins each having the amino acid sequence shown in SEQ ID NO: 2 were obtained.

200 μl of each purified preparation obtained by the above method was dispensed into 1 ml of reaction buffer (20 mM Tris-HCl, 50 mM NaCl, 2 mM CaCl₂ pH 7.4) and then 2 μl of enterokinase (Novagen) was added. The preparation was left to stand at room temperature overnight for reaction, His tag was cleaved, and then purification was performed according to the attached protocols using an Enterokinase Cleavage Capture Kit (Novagen). Next, 1.2 ml of each purified preparation obtained by the above method was substituted with physiological phosphate buffer (Nissui Pharmaceutical Co., Ltd.) using ultrafiltration NANOSEP 10K OMEGA (PALL). Sterilized filtration was performed using 0.22-μm HT Tuffryn Acrodisc (PALL) and then the resultants were used for the following experiments.

### Example 4: Preparation of Monoclonal Antibody Against CAPRIN-1

The antigen protein (human CAPRIN-1) (100 μg) shown in SEQ ID NO: 2 prepared in Example 3 was mixed with a MPL+TDM adjuvant (Sigma) in an amount equivalent to that of the antigen protein. The mixture was used as an antigen solution per mouse. The antigen solution was administered intraperitoneally to 6-week-old Balb/c mice (Japan SLC Inc.) and then further administered 3 times or 24 times every week for completion of immunization. Spleen was removed on day 3 after the final immunization and then ground in between two sterilized glass slides. Each resultant was washed with PBS (−) (Nissui) and then centrifuged at 1500 rpm for 10 minutes, so that a procedure to remove supernatants was repeated 3 times. Thus, spleen cells were obtained. The thus obtained spleen cells were mixed with the mouse myeloma cell SP2/0 (purchased from ATCC) at a ratio of 10:1. The PEG solution prepared by mixing 200 μl of RPMI1640 medium containing 10% FBS heated at 37° C. and 800 μl of PEG1500 (Boehringer) was added to the cells. The solution was left to stand for 5 minutes for cell fusion. Centrifugation was performed at 1700 rpm for 5 minutes to remove supernatants. Cells were suspended in 150 ml of RPMI1640 medium (HAT selective medium) containing 15% FBS, to which 2% equivalent of HAT solution (Gibco) had been added and then seeded onto fifteen 96-well plates (Nunc) at 100 μl per well. Cells were cultured for 7 days under conditions of 37° C. and 5% CO₂, so that hybridomas resulting from fusion of spleen cells to myeloma cells were obtained.

Hybridomas were selected using as an indicator the binding affinity of the antibody produced by the thus prepared hybridomas for the CAPRIN-1 protein. The CAPRIN-1 protein solution (1 μg/ml) prepared in Example 3 was added at 100 μl per well of 96-well plates and then left to stand at 4° C. for 18 hours. Each well was washed 3 times with PBS-T, 0.5% Bovine Serum Albumin (BSA) solution (Sigma) was added at 400 μl per well, and then the plates were left to stand at room temperature for 3 hours. The solution was removed and then each well was washed 3 times with 400 μl of PBS-T. Each culture supernatant of the hybridomas obtained above was added at 100 μl per well and then left to stand at room temperature for 2 hours. Each well was washed 3 times with PBS-T, an HRP-labeled anti-mouse IgG (H+L) antibody (Invitrogen) diluted 5000-fold with PBS was added at 100 μl per well and then left to stand at room temperature for 1 hour. Each well was washed 3 times with PBS-T, a TMB substrate solution (Thermo) was added at 100 μl per well and then left to stand for 15-30 minutes, so that a color reaction was performed. After color development, 1N sulfuric acid was added at 100 μl per well to stop the reaction. Absorbance at 450 nm and absorbance at 595 nm were measured using an absorption spectrometer. As a result, a plurality of hybridomas producing antibodies with high absorbances were selected.

The thus selected hybridomas were added at 0.5 hybridomas per well of 96-well plates and then cultured. After 1 week, hybridomas forming single colonies in wells were observed. Cells in these wells were further cultured. Hybridomas were selected using as an indicator the binding affinity (of the antibody produced by cloned hybridomas) for the CAPRIN-1 protein. The CAPRIN-1 protein solution (1 μg/ml) prepared in Example 3 was added at 100 μl per well of 96-well plates and then left to stand at 4° C. for 18 hours. Each well was washed 3 times with PBS-T, a 0.5% BSA solution was added at 400 μl per well, and then left to stand at room temperature for 3 hours. The solution was removed and then each well was washed 3 times with 400 μl of PBS-T. Each culture supernatant of the hybridomas obtained above was added at 100 μl per well and then left to stand at room temperature for 2 hours. Each well was washed 3 times with PBS-T, an HRP-labeled anti-mouse IgG (H+L) antibody (Invitrogen) diluted 5000-fold with PBS was added at 100 μl per well and then left to stand at room temperature for 1 hour. Each well was washed 3 times with PBS-T, a TMB substrate solution (Thermo) was added at 100 μl per well and then left to stand for 15-30 minutes, so that a color reaction was performed. After color development, 1N sulfuric acid was added at 100 μl per well to stop the reaction. Absorbance at 450 nm and absorbance at 595 nm were measured using an absorption spectrometer. As a result, 150 hybridoma cell lines producing monoclonal antibodies exerting reactivity with the CAPRIN-1 protein were obtained.

Next, from among these monoclonal antibodies, monoclonal antibodies exerting reactivity with the surfaces of breast cancer cells expressing CAPRIN-1 were selected. Specifically, 10⁶ cells of the MDA-MB-231V human breast cancer cell line were subjected to centrifugation with a 1.5-ml microcentrifugal tube. The supernatant (100 μl) of each hybridoma above was added and then left to stand on ice for 1 hour. After washing with PBS, an FITC-labeled goat anti-mouse IgG antibody (Invitrogen) diluted 500-fold with PBS containing 0.1% FBS was added and then left to stand on ice for 1 hour. After washing with PBS, fluorescence intensity was measured using FACSCalibur (Becton, Dickinson and Company). Meanwhile, a procedure similar to the above was performed using untreated serum of 6-week-old Balb/c mice diluted 500-fold with a hybridoma culture medium instead of the antibody so that a control was prepared. As a result, 11 monoclonal antibodies (#1 to #11) having fluorescence intensity stronger than that of the control; that is, reacting with the surfaces of breast cancer cells were selected.

### Example 5: Characterization of Selected Antibodies

### (1) Cloning of an Anti-CAPRIN-1 Monoclonal Antibody Variable Region Gene

mRNAs were extracted from hybridoma cell lines producing the 11 monoclonal antibodies selected in Example 4. The heavy-chain variable (VH) region gene and the light-chain variable (VL) region gene for every anti-CAPRIN-1 monoclonal antibody were obtained by RT-PCR using primers specific to a mouse FR1-derived sequence and a mouse FR4-derived sequence. For sequencing, the genes were separately cloned into pCR2.1 vectors (Invitrogen).

### (1)-1 RT-PCR

mRNA was prepared from each hybridoma cell line (10⁶ cells) using an mRNA micro purification kit (GE Healthcare). Each obtained mRNA was subjected to reverse transcription using a SuperScriptII 1st strand synthesis kit (Invitrogen) for cDNA synthesis. The above procedures were carried out according to the protocols attached to the kits.

Each obtained cDNA was used for antibody gene amplification by PCR.

In order to obtain the VH region gene, a primer specific to a mouse heavy-chain FR1 sequence (SEQ ID NO: 130) and a primer specific to a mouse heavy-chain FR4 sequence (SEQ ID NO: 131) were used. In addition, in order to obtain the VL region gene, a primer specific to a mouse light-chain FR1 sequence (SEQ ID NO: 132) and a primer specific to a mouse light-chain FR4 sequence (SEQ ID NO: 133) were used. These primers were designed with reference to Jones, S. T. and Bending, M. M. Bio/Technology 9, 88-89 (1991). For PCR, Ex-taq (Takara Bio Inc.) was used. Each cDNA sample was mixed with a 10×EX Taq Buffer (5 μl), dNTPs Mixture (2.5 mM)(4 μl), primers (1.0 μM)(2 μl each), and Ex Taq (5U/μl)(0.25 μl). The total volume was adjusted to 50 μl with sterilized water. PCR was carried out under conditions comprising, after treatment at 94° C. for 2 minutes, 30 cycles of a combination of denaturation at 94° C. for 1 minute, annealing at 58° C. for 30 seconds, and elongation reaction at 72° C. for 1 minute.

### (1)-2 Cloning

Each PCR product obtained above was subjected to agalose gel electrophoresis, followed by excision of DNA bands of the VH region and the VL region. DNA was purified using a QIAquick Gel purification kit (QIAGEN) according to the protocols attached to the kit. Each purified DNA was cloned into a pCR2.1 vector using a TA cloning kit (Invitrogen). Each DNA-ligated vector was transformed into DH5a competent cells (TOYOBO) according to a conventional method. Each transformant (10 clones) was cultured overnight in a medium (100 μg/ml ampicillin) at 37° C. The obtained plasmid DNA was purified using a Qiaspin Miniprep kit (QIAGEN).

### (1)-3 Sequencing

Gene sequence analysis of the VH region and the VL region in each plasmid obtained above was carried out using an M13 forward primer (SEQ ID NO: 134) and an M13 reverse primer (SEQ ID NO: 135) with a fluorescent sequencer (ABI; DNA sequencer 3130XL) and a BigDye terminater Ver. 3.1 cycle sequencing kit (ABI) in accordance with the protocols attached to the kit. As a result, each gene sequence (identical in 10 clones) was determined.

The obtained amino acid sequences of monoclonal antibody heavy-chain variable regions are shown in SEQ ID NO: 43, SEQ ID NO: 73, SEQ ID NO: 83, SEQ ID NO: 93, SEQ ID NO: 103, SEQ ID NO: 113, and SEQ ID NO: 123. The obtained amino acid sequences of light-chain variable regions are shown in SEQ ID NO: 47, SEQ ID NO: 53, SEQ ID NO: 58, SEQ ID NO: 63, SEQ ID NO: 68, SEQ ID NO: 77, SEQ ID NO: 87, SEQ ID NO: 97, SEQ ID NO: 107, SEQ ID NO: 117, and SEQ ID NO: 127.

Specifically, a monoclonal antibody #1 comprises the heavy-chain variable region of SEQ ID NO: 43 and the light-chain variable region of SEQ ID NO: 47. A monoclonal antibody #2 comprises the heavy-chain variable region of SEQ ID NO: 43 and the light-chain variable region of SEQ ID NO: 53. A monoclonal antibody #3 comprises the heavy-chain variable region of SEQ ID NO: 43 and the light-chain variable region of SEQ ID NO: 58. A monoclonal antibody #4 comprises the heavy-chain variable region of SEQ ID NO: 43 and the light-chain variable region of SEQ ID NO: 63. A monoclonal antibody #5 comprises the heavy-chain variable region of SEQ ID NO: 43 and the light-chain variable region of SEQ ID NO: 68. A monoclonal antibody #6 comprises the heavy-chain variable region of SEQ ID NO: 73 and the light-chain variable region of SEQ ID NO: 77. A monoclonal antibody #7 comprises the heavy-chain variable region of SEQ ID NO: 83 and the light-chain variable region of SEQ ID NO: 87. A monoclonal antibody #8 comprises the heavy-chain variable region of SEQ ID NO: 93 and the light-chain variable region of SEQ ID NO: 97. A monoclonal antibody #9 comprises the heavy-chain variable region of SEQ ID NO: 103 and the light-chain variable region of SEQ ID NO: 107. A monoclonal antibody #10 comprises the heavy-chain variable region of SEQ ID NO: 113 and the light-chain variable region of SEQ ID NO: 117. A monoclonal antibody #11 comprises the heavy-chain variable region of SEQ ID NO: 123 and the light-chain variable region of SEQ ID NO: 127.

(2) Expression of CAPRIN-1 on the Cell Surfaces of Different Cells Caused with the Use of the Obtained Monoclonal Antibodies

Next, it was examined whether or not the CAPRIN-1 protein was expressed on cell surfaces of 7 types of breast cancer cell lines (MDA-MB-157, T47D, MRK-nu-1, MDA-MB-231V, BT20, SK-BR-3, and DA-MB-231T) in which CAPRIN-1 gene expression had been confirmed, 3 types of other breast cancer cell lines (MDA-MB-231C, MCF-7, and ZR75-1), 6 types of glioma cell lines (T98G, SNB19, U251, and U87G), 3 types of kidney cancer cell lines (Caki-1, Caki2, and A498), 1 type of a gastric cancer cell line (MKN45), 1 type of a colon cancer cell line (Caco2), 3 types of lung cancer cell lines (A549, QG56, and PC8), and 3 types of leukemia cell lines (Namalwa, BDCM, and RPI1788). Each cell line (10⁶ cells) was centrifuged in a 1.5-ml microcentrifugal tube. The hybridoma supernatants (100 μl each) containing monoclonal antibodies #1 to #10 against CAPRIN-1 prepared in Example 4 reacting to cancer cell surfaces were separately added thereto and then left to stand on ice for 1 hour. After washing with PBS, each resultant was suspended in an FITC-labeled goat anti-mouse IgG antibody (Invitrogen Corporation) diluted 500-fold with PBS containing 0.1% FBS and then left to stand on ice for 1 hour. After washing with PBS, fluorescence intensity was measured using FACSCalibur (Becton, Dickinson and Company). Meanwhile, a procedure similar to the above was performed using, as a control, the control antibody prepared in (5) above instead of the hybridoma supernatants containing monoclonal antibodies #1 to #11 against CAPRIN-1, so that a control was prepared. As a result, fluorescence intensity was found to be at least 30% stronger in all cells to which the monoclonal antibodies #1 to #11 had been added than that in control cells. Specifically, the following increases in fluorescence intensity were confirmed when, for example, the monoclonal antibody #9 was used: MDA-MB-157: 211%; T47D: 145%; MRK-nu-1: 123%; MDA-MB-231V: 251%; BT20: 168%; and MDA-MB-231T: 94%. Based on the above, it was confirmed that the CAPRIN-1 protein was expressed on the cell surfaces of the above human cancer cell lines. In addition, the rate of increase in fluorescence intensity is represented by the rate of increase in mean fluorescence intensity (MFI value) in cells. It was calculated by the following equation.

Rate of increase in mean fluorescence intensity (rate of increase in fluorescence intensity) (%)=((MFI value of cells reacted with an anti-human CAPRIN-1 antibody)−(control MFI value))/(control MFI value)×100

### (3) Antitumor Effects (ADCC Activity) of Antibodies Against CAPRIN-1 Upon Cancer Cells

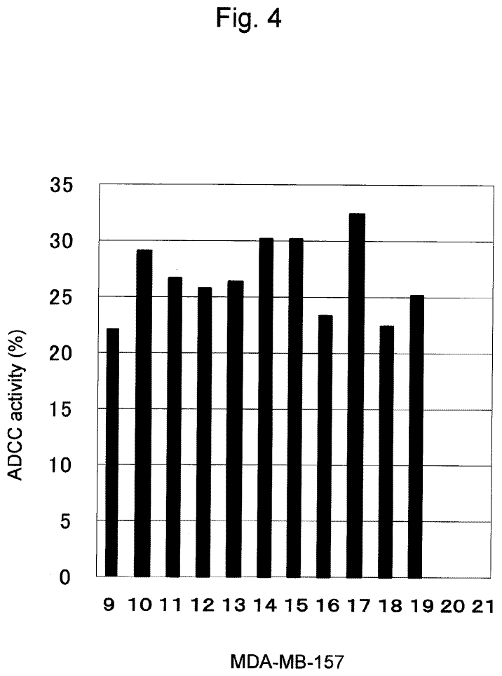

The above selected monoclonal antibodies #1 to #11 against CAPRIN-1 were evaluated in terms of cytotoxic activity (ADCC activity) to cancer cells. The hybridomas producing monoclonal antibodies #1 to #11 were cultured using a hybridoma SFM medium (Invitrogen). Each obtained supernatant was purified using Hitrap ProteinA Sepharose FF (GE Healthcare), followed by substitution with PBS (−) and purification with a 0.22-μm filter (Millipore). Each resultant was used as an antibody for activity determination. The human breast cancer MDA-MB-157 cell line (10⁶ cells) was collected into a 50-ml centrifugal tube. Chromium 51 (100 μCi) was added thereto, followed by incubation at 37° C. for 2 hours. Thereafter, cells were washed 3 times with an RPMI1640 medium containing 10% FBS. The cells were added to wells (10³ cells per well) in 96-well V-bottom plates. Thus, target cells were prepared. The above purified antibodies were added thereto (1 μg per well). Further, mouse lymphocytes separated from mouse spleen (2×10⁵ cells) were added thereto, followed by culture under conditions of 37° C. and 5% CO₂ for 4 hours. After culture, the level of chromium (Cr) 51 released from damaged tumor cells in each culture supernatant was determined. Then, ADCC activity of each polyclonal antibody against a human CAPRIN-1-derived peptide to cancer cells was calculated. As a result, all monoclonal antibodies #1 to #11 exhibited ADCC activity against MDA-MB-157 (20% or more). Specifically, Specifically, for example, the following cytotoxic activity results were obtained: #1: 22.1%; #2: 29.1%; #6: 30.2%; and #9: 32.4% (see FIG. 4). Meanwhile, no cytotoxic activity was confirmed in a case in which a procedure similar to the above was performed using the monoclonal antibody reactive to a CAPRIN-1 protein itself but not to cancer cell surfaces prepared in Example 4 (see FIG. 4). The above results showed that the obtained anti-CAPRIN-1 monoclonal antibodies (#1 to #11) damaged CAPRIN-1-expressing cancer cells by exhibiting ADCC activity.

### (4) Antitumor Effects (CDC Activity) of Antibodies Against CAPRIN-1 Upon Cancer Cells

The above selected monoclonal antibodies #1 to #11 against CAPRIN-1 were evaluated in terms of cytotoxic activity (CDC activity) to cancer cells. Blood collected from rabbits by blood sampling was added to an Eppendorf tube and then left to stand at room temperature for 60 minutes, followed by centrifugation at 3000 rpm for 5 minutes. Thus, serum for CDC activity determination was prepared. The human breast cancer MDA-MB-231V cell line (10⁵ cells) was collected into a 50-ml centrifugal tube. Chromium 51 (100 μCi) was added thereto, followed by incubation at 37° C. for 2 hours. Thereafter, cells were washed 3 times with an RPMI medium containing 10% FBS and then suspended in an RPMI containing 50% rabbit serum prepared above. The cells were added to wells (10³ cells per well) in 96-well V-bottom plates. The antibodies #1 to #11 obtained in (3) above were separately added to wells (1 μg per well), followed by culture under conditions of 37° C. and 5% CO₂ for 4 hours. After culture, the level of chromium (Cr) 51 released from damaged tumor cells in each culture supernatant was determined. The CDC activity against MDA-MB-231V exhibited by the anti-CAPRIN-1 monoclonal antibody in each hybridoma supernatant was calculated. As a result, all monoclonal antibodies #1 to #11 exhibited CDC activity (30% or more). Meanwhile, no cytotoxic activity was confirmed in a case in which a procedure similar to the above was performed using the monoclonal antibody reactive to a CAPRIN-1 protein itself but not to cancer cell surfaces prepared in Example 4 (see FIG. 4). Accordingly, it has been revealed that the monoclonal antibodies against CAPRIN-1 (#1 to #11) can damage CAPRIN-1-expressing tumor cells also by exhibiting CDC activity.

### Example 6: In Vivo Antitumor Effects of Anti-CAPRIN-1 Monoclonal Antibodies Upon Mice

Next, in vivo antitumor effects of the obtained monoclonal antibodies #1 to #11 against CAPRIN-1 upon tumor-bearing mice were evaluated. Antibodies used in this Example were obtained by subjecting the supernatant of each hybridoma to column purification in the manner described above.

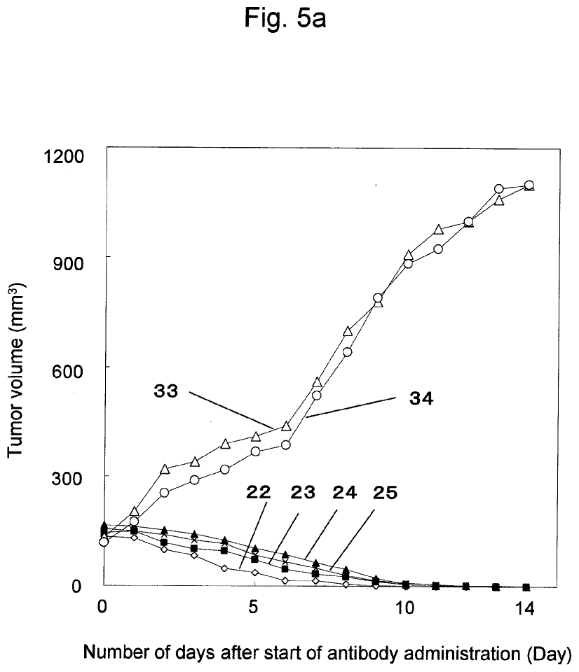

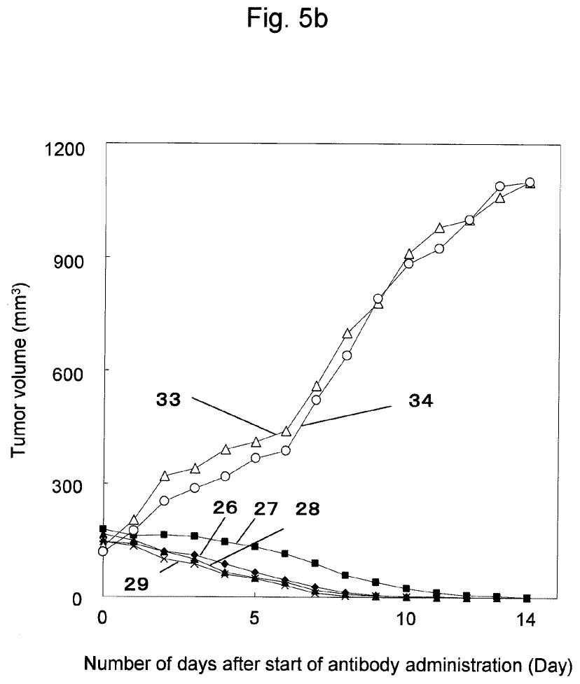

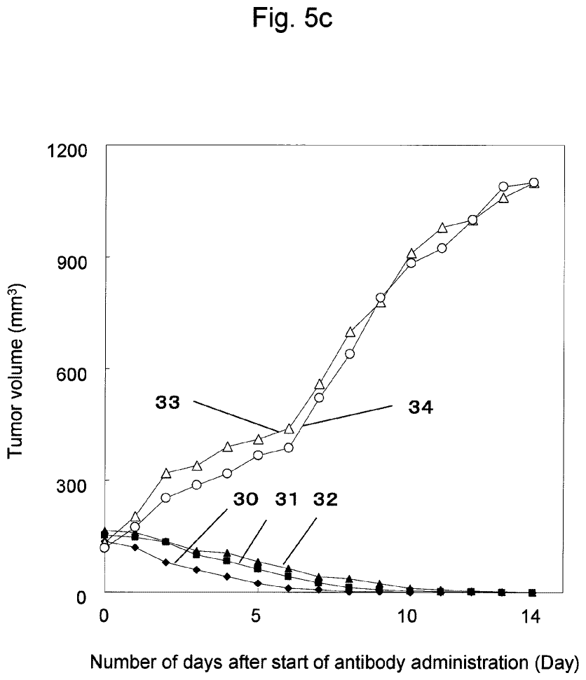

Antitumor effects of the monoclonal antibodies #1 to #11 against CAPRIN-1 were examined using tumor-bearing mice into which a mouse-derived cancer cell line expressing CAPRIN-1 had been transplanted. CT26 cells (purchased from ATCC) were subcutaneously transplanted into the dorsal portions of 70 Balb/c mice (Japan SLC, Inc.)(10⁶ cells per mouse). Each tumor was allowed to grow until the diameter thereof became approximately 7 mm. The tumor-bearing mice (60 out of 70) were subjected to intraperitoneal administration of monoclonal antibodies #1 to #11 against CAPRIN-1 and one type of the monoclonal antibody (reactive to the CAPRIN-1 protein itself but not to cancer cell surfaces) prepared in Example 4 (5 mice per antibody) at a dose of 300 μg (300 μl) per mouse. Thereafter, each antibody was intraperitoneally administered in the same dose to the relevant tumor-bearing mice 3 times in total during 2 days. The tumor size was measured every day for observation of antitumor effects. The 10 remaining tumor-bearing mice were subjected to administration of PBS (−) instead of an antibody. The group of these mice was designated as a control group. As a result of observation of antitumor effects, in the case of the test group to which monoclonal antibodies #1 to #11 against CAPRIN-1 had been administered, tumor regression occurred to such an extent that the tumor volume at the start of antibody administration (100%) decreased to 50% by Day 4, approximately 10% by Day 6, and several percents by Day 8. Substantially complete tumor regression took place from Days 11 to 14 (see FIG. 5). On the other hand, in the control group, the tumor volume increased to approximately 260%, 350%, 550%, and 800% of the original volume by Days 4, 6, 8, and 11, respectively (see FIG. 5). In addition, in the group of mice to which the monoclonal antibody (reactive to the CAPRIN-1 protein itself but not to cancer cell surfaces) had been administered, antitumor effects could not be exhibited and tumor growth occurred as in the control group. The results indicate that the obtained monoclonal antibodies #1 to #11 against CAPRIN-1 exhibit strong in vivo antitumor effects upon cancer cells expressing CAPRIN-1. In addition, the tumor size was obtained by calculating the tumor volume by the following formula: long diameter×short diameter×short diameter×0.5.

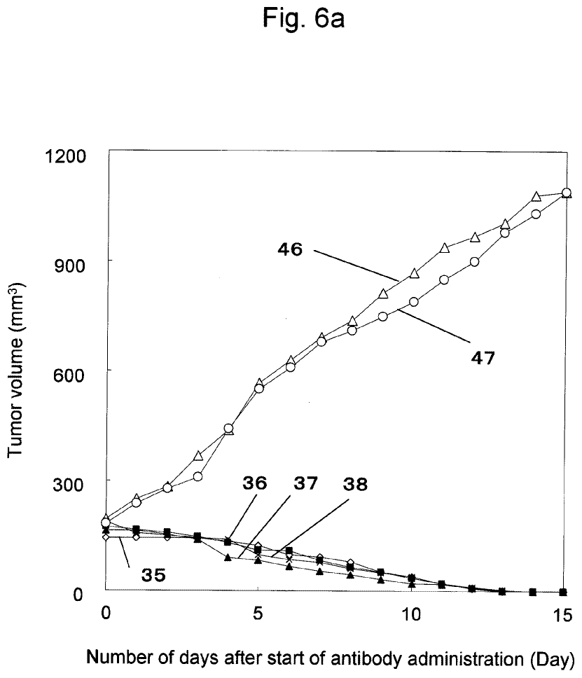

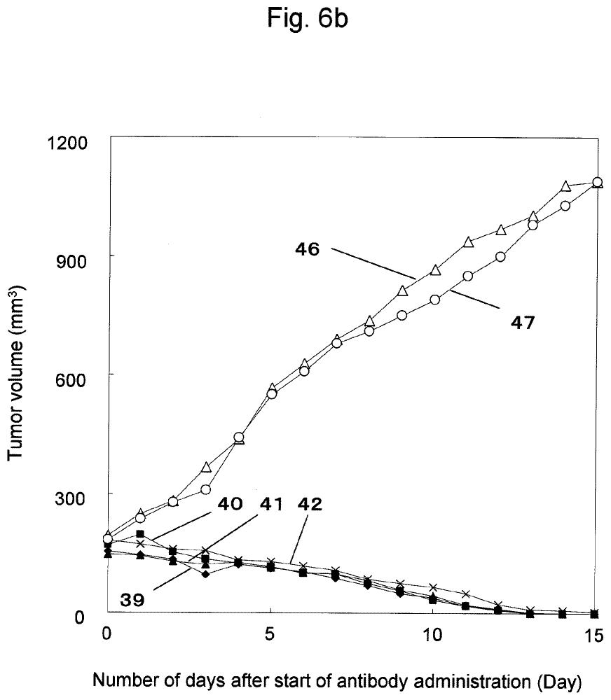

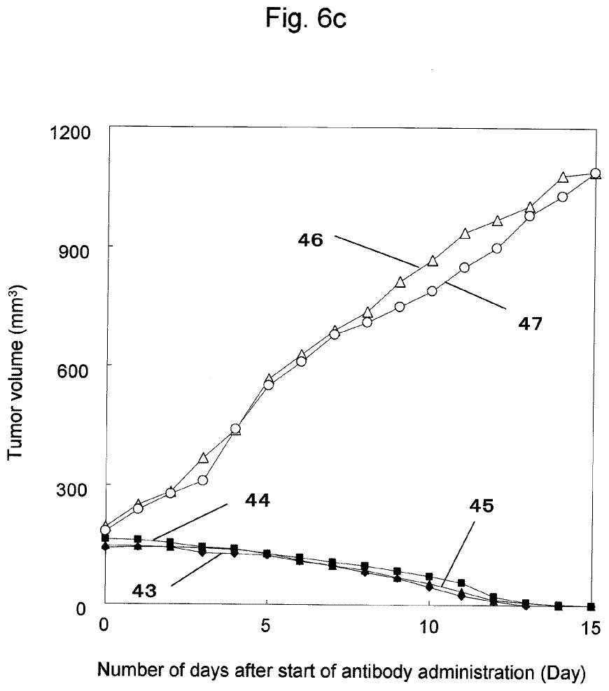

Further, monoclonal antibodies #1 to #11 against CAPRIN-1 were administered in the manner described above to tumor-bearing mice (Balb/c) into which mouse N1E cancer cells (purchased from ATCC) had been transplanted. This resulted in complete tumor regression by Day 15 after antibody administration. On the other hand, in the control group, the tumor volume increased to as high as approximately 950% of the original volume (see FIG. 6).

### Example 7: Identification of a Peptide in CAPRIN-1 Protein, to which an Antibody Against CAPRIN-1 Reacting to Cancer Cell Surface Binds

With the use of monoclonal antibodies #1 to #11 against CAPRIN-1, reacting with the surfaces of cancer cells (obtained above), partial sequences in the CAPRIN-1 protein to be recognized by these monoclonal antibodies were identified.

First, DTT (Fluka) was added to 100 μl of a solution prepared by dissolving a recombinant CAPRIN-1 protein at a concentration of 1 μg/μl with PBS to a final concentration of 10 mM, followed by 5 minutes of reaction at 95° C., so that reduction of disulfide bonds within the CAPRIN-1 protein was performed. Next, iodoacetamide (Wako Pure Chemical Industries, Ltd.) with a final concentration of 20 mM was added and then an alkylation reaction was performed for thiol groups at 37° C. for 30 minutes under shading conditions. Fifty (50) μg each of monoclonal antibodies #1 to #11 against CAPRIN-1 was added to 40 μg of the thus obtained reduced-alkylated CAPRIN-1 protein, the volume of the mixture was adjusted to 1 mL of 20 mM phosphate buffer (pH 7.0), and then the mixture was left to react overnight at 4° C. while stirring and mixing each mixture.

Next, trypsin (Promega) was added to a final concentration of 0.2 μg. After 1 hour, 2 hours, 4 hours, and then 12 hours of reaction at 37° C., the resultants were mixed with protein A-glass beads (GE), which were subjected in advance to blocking with PBS containing 1% BSA (Sigma) and then to washing with PBS, in 1 mM calcium carbonate and NP-40 buffer (20 mM phosphate buffer (pH 7.4), 5 mM EDTA, 150 mM NaCl, and 1% NP-40), followed by 30 minutes of reaction.

The reaction mixtures were each washed with 25 mM ammonium carbonate buffer (pH 8.0) and then antigen-antibody complexes were eluted using 100 μl of 0.1% formic acid. LC-MS analysis was conducted for eluates using Q-TOF Premier (Waters-MicroMass) according to the protocols attached to the instrument.

As a result, the polypeptide of SEQ ID NO: 136 was identified as a partial sequence of CAPRIN-1, which was recognized by all of the monoclonal antibodies #1 to #11 against CAPRIN-1. Furthermore, the peptide of SEQ ID NO: 137 was identified as a partial sequence in the polypeptide of SEQ ID NO: 136 above, which peptide was recognized by the monoclonal antibodies #2 to #5, #6 to #8, and #10. It was further revealed that the monoclonal antibodies #2 to #5 recognized the peptide of SEQ ID NO: 138 that was a partial sequence peptide of the peptide of SEQ IS NO: 137.

### INDUSTRIAL APPLICABILITY

The antibodies of the present invention are useful for treatment and/or prevention of cancers.

This description includes all or part of the contents as disclosed in the description and/or drawings of Japanese Patent Application No. 2009-087285, to which the present application claims the priority. In addition, all publications, patents, and patent applications cited herein are incorporated herein by reference in their entirety.

### FREE TEXT OF SEQUENCE LISTING

Primers: SEQ ID NOS: 31 to 39 and 130 to 135

1. A method for delivering an antitumor agent to a cancer cell, comprising administering an antibody-antitumor agent conjugate or an antibody fragment-antitumor agent conjugate that binds a CAPRIN-1 protein to a subject;
wherein the cancer cell is a CAPRIN-1 expressing cancer cell; and
wherein the antibody or the antibody fragment specifically binds the extracellular region of the CAPRIN-1 protein expressed on the cell surface of the CAPRIN-1 expressing cancer cell.

2. The method according to claim 1, wherein the CAPRIN-1 protein consists of the amino acid sequence shown in any one of the even numbered SEQ ID Nos: 2 to 30.

3. The method according to claim 1, wherein the antibody is a monoclonal antibody or polyclonal antibody.

4. The method according to claim 1, wherein the antibody is a human antibody, a humanized antibody, a chimeric antibody, a single-chain antibody, or a bispecific antibody.

5. The method according to claim 1, wherein the antitumor agent is paclitaxel, doxorubicin, daunorubicin, cyclophosphamide, methotrexate, 5-fluorouracil, thiotepa, busulfan, improsulfan, piposulfan, benzodopa, carboquone, meturedopa, uredopa, altretamine, triethylenemelamine, triethylenephosphoramide, triethilenethiophosphoramide, trimethylolomelamine, bullatacin, bullatacinone, camptothecin, bryostatin, callystatin, cryptophycin 1, cryptophycin 8, dolastatin, duocarmycin, eleutherobin, pancratistatin, sarcodictyin, spongistatin, chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard, carmustine, chlorozotocin, fotemustine, lomustine, nimustine, ranimustine, calicheamicin, dynemicin, clodronate, esperamicin, aclacinomycin, actinomycin, authramycin, azaserine, bleomycin, cactinomycin, carabicin, carminomycin, carzinophilin, chromomycin, dactinomycin, detorbicin, 6-diazo-5-oxo-L-norleucine, epirubicin, esorubicin, idarubicin, marcellomycin, mitomycin C, mycophenolic acid, nogalamycin, olivomycin, peplomycin, potfiromycin, puromycin, quelamycin, rodorubicin, streptonigrin, streptozocin, tubercidin, ubenimex, zinostatin, zorubicin, denopterin, pteropterin, trimetrexate, fludarabine, 6-mercaptopurine, thiamiprine, ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone, aminoglutethimide, mitotane, trilostane, frolinic acid, aceglatone, aldophosphamide glycoside, aminolevulinic acid, eniluracil, amsacrine, bestrabucil, bisantrene, edatraxate, defofamine, demecolcine, diaziquone, elfornithine, elliptinium acetate, epothilone, etoglucid, lentinan, lonidamine, maytansine, ansamitocine, mitoguazone, mitoxantrone, mopidanmol, nitraerine, pentostatin, phenamet, pirarubicin, losoxantrone, podophyllinic acid, 2-ethylhydrazide, procarbazine, razoxane, rhizoxin, schizophyllan, spirogermanium, tenuazonic acid, triaziquone, roridine A, anguidine, urethane, vindesine, dacarbazine, mannomustine, mitobronitol, mitolactol, pipobroman, gacytosine, docetaxel, gemcitabine, 6-thioguanine, cisplatin, oxaliplatin, carboplatin, vinblastine, etoposide, vincristine, vinorelbine, novantrone, teniposide, edatrexate, daunomycin, aminopterin, xeloda, ibandronate, irinotecan, topoisomerase inhibitor, difluoromethylornithine (DMFO), retinoic acid, capecitabine, or pharmacologically acceptable salts or derivatives thereof.
